(19) United States
(12) Patent Application Publication
(10) Pub. No.: US 2023/0060351 A1
(43) Pub. Date: Mar. 2, 2023

(54) A METHOD OF ENGINEERING NATURAL KILLER CELLS TO TARGET CD70-POSITIVE TUMORS

(71) Applicant: BOARD OF REGENTS, THE UNIVERSITY OF TEXAS SYSTEM, Austin, TX (US)

(72) Inventor: Katy REZVANI, Houston, TX (US)

(21) Appl. No.: 17/758,094

(22) PCT Filed: Jan. 7, 2021
(86) PCT No.: PCT/US2021/012510

§ 371 Date: Jun. 28, 2022

Related U.S. Application Data
(60) Provisional application No. 62/958,563, filed on Jan. 8, 2020.

(51) Int. Cl. C07K 16/28; C07K 14/715; C07K 14/725; C12N 5/0783

(52) U.S. Cl. CPC C07K 16/2875; C07K 14/715; C07K 14/7051; C12N 5/0646; C07K 2317/622; C07K 2317/24; C07K 2319/03

(57) ABSTRACT

Embodiments of the disclosure include methods and compositions related to targeting of CD70-expressing cells with NK cells specifically engineered to bind the CD70 antigen. In particular embodiments, NK cells that are manipulated to expressing CD70-targeting engineered receptors, such as CARs, are utilized to target cancers that express CD70. In certain embodiments, vectors that express the CD70-targeting CARs also express particular a suicide gene and/or one or more particular cytokines.

This application claims priority to U.S. Provisional Patent Application Ser. No. 62/958,563, filed Jan. 8, 2020, which is incorporated by reference herein in its entirety.

### TECHNICAL FIELD

Embodiments of the disclosure include at least the fields of cell biology, molecular biology, immunology, and medicine, including cancer medicine.

### BACKGROUND

Genetic reprogramming of Natural Killer (NK) cells for adoptive cancer immunotherapy has clinically relevant applications and benefits such as 1) innate anti-tumor surveillance without prior need for sensitization; 2) allogeneic efficacy without graft versus host reactivity; and 3) direct cell-mediated cytotoxicity and cytolysis of target tumors. Human NK cell development and acquisition of self-tolerance, alloreactivity, and effector functions is an adaptive process of licensing, calibration, and arming. At the molecular level, specific activating and inhibitory receptors direct NK cellular functions by aggregating, balancing, and integrating extracellular signals into distinct effector functions. The functional activity of NK cells and responsiveness to extrinsic stimuli follow the ‘rheostat’ model of continuous education and thus amenable to reprogramming. Genetic modification of NK cells to redirect their effector functions is an effective method to harness their cytotoxic capability to kill tumor cells.

The present disclosure concerns improvements in cell therapy and adoptive cell therapy directed at Cluster of Differentiation (CD70)-positive cancers.

### BRIEF SUMMARY

Embodiments of the disclosure encompass methods and compositions related to engineered cellular receptors that target CD70 (also known as CD27 ligand, CD27LG, and TNFSF7, for example). In specific embodiments, the engineered receptors that target CD70 are in the form of polynucleotides, polypeptides, or are comprised on the surface of cells of any kind, including immune cells. In specific cases, the cells are immune cells, and in certain embodiments the immune cells are NK cells, NK T cells, invariant NKT cells, gamma delta T cells, regulatory T cells, B cells, macrophages, mesenchymal stromal cells (MSCs), dendritic cells, and so forth from any source. In certain embodiments, reprogrammed NK cells from cord blood (CB-NK) are encompassed for targeting tumors expressing CD70 molecules.

CD70 is utilized as a target antigen for methods and compositions because it is expressed on many cancers, including acute myeloid leukemia (AML), lymphoma, lung cancer, melanoma, breast cancer, glioblastoma, mesothelioma, head and neck cancer, renal cancer, multiple myeloma, and pancreatic tumors, as examples. Expression of CD70 in normal tissue is limited to a subset of T cells and dendritic cells (DC).

Embodiments of the disclosure encompass a variety of novel, specific CAR constructs incorporating CD70 scFv heterologously fused to one or more signaling domains (including, for example, those comprising cytoplasmic portions of CD247 (also known as CD3) and one or more of CD28, DAP10, DAP12, and NKG2D. The scFv may comprise a fusion of the variable fragments derived from the heavy (V_{H}) and light (VL) chains of a murine antibody with specificity for human CD70 antigen, in some cases. The vector also may comprise one or more cytokine genes, including the gene to produce human interleukin 15 (IL-15), IL-2, IL-21, IL-12, IL-7, and/or IL-18 that aids in the survival and maintenance of the NK cells. As one example, CB-NK cells, thus modified, comprise a vector encoding CD70 scFv in a CAR that includes CD28 and CD3z in addition to IL15 that is produced as a separate molecule from the CAR.

Although in some embodiments the methods and compositions are utilized to treat individuals with CD70-positive cancers, in other cases the methods and compositions are utilized to ablate CD70-expressing (non-cancerous) immunoregulatory cells, such as T regulatory cells (Tregs) as checkpoints. In specific embodiments, there are methods of targeting CD70-expressing non-cancerous cells in an individual comprising delivering to the individual an effective amount of CD70 CAR-expressing cells.

Particular embodiments of the disclosure allow for the use of off-the-shelf immune cells, including at least NK cells, that are allogeneic with respect to a recipient individual, that target CD70-positive cells of any kind, and that also may or may not be transduced to express one or more cytokines, such as IL15, IL-2, IL21, IL-12, IL-7, and/or IL-18.

In specific embodiments of the disclosure, expression of one or more endogenous genes in the immune cell has been modified, for example the expression may be partially or fully reduced in expression. Although the modification may occur by any means, in specific embodiments expression of the one or more genes has been modified, such as by being reduced in expression levels, and this may occur by any suitable means including at least CRISPR. Merely as examples, the endogenous gene may be selected from the group consisting of NKG2A, SIGLEC-7, LAGS, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, CD5, CD7, CTLA-4, TDAG8, CD38, and a combination thereof.

In one embodiment, there is an expression construct comprising sequence that encodes a CD70-specific engineered receptor and that encodes one or both of the following: (a) a suicide gene; and (b) a cytokine. In specific cases, the CD70-specific engineered receptor is a chimeric antigen receptor (CAR) or a T cell receptor. The CD70-specific CAR may comprise a scFv having a heavy chain and a light chain, and wherein the heavy chain in the sequence that encodes the CAR is upstream of the light chain in a 5′ to 3′ direction. In other cases, the CD70-specific CAR comprises a scFv having a heavy chain and a light chain, and wherein the heavy chain in the sequence that encodes the CAR is downstream of the light chain in a 5′ to 3′ direction. In any case herein, the CD70-specific CAR does or does not comprise a codon optimized scFv. In any case herein, the CD70-specific CAR does or does not comprise a humanized scFv. In any case herein, the CD70-specific CAR does or does not comprise a signaling peptide, such as one from CD8alpha, Ig heavy chain, or granulocyte-macrophage colony-stimulating factor receptor or a signal peptide derived from one or more other surface receptors. In particular embodiments, the CD70-specific CAR comprises one or more costimulatory domains, such as one or more costimulatory domains selected from the group consisting of CD28, CD27, OX-40 (CD134), DAP10, DAP12, 4-1BB (CD137), CD40L, 2B4, DNAM, CS1, CD48, NKG2D, NKp30, NKp44, NKp46, NKp80, or a combination thereof.

Any CD70-specific CAR may or may not comprise CD3zeta and/or a hinge between the scFv and a transmembrane domain. In specific cases, the hinge is CD8-alpha hinge, the hinge comprises an artificial spacer comprised of Gly3, or the hinge comprises CH1, CH2, and/or CH3 domains of IgGs. In specific embodiments, the cytokine is IL-15, IL-12, IL-2, IL-18, IL-21, IL-7, or a combination thereof. In cases wherein a suicide gene is employed, the suicide gene may be a mutant TNF-alpha (such as an engineered nonsecretable mutant), inducible caspase 9, HSV-thymidine kinase, CD19, CD20, CD52, or EGFRv3.

Embodiments of the disclosure include expression constructs that comprise any one or more of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, or SEQ ID NO:13.

Embodiments of the disclosure include immune cells of any kind comprising any expression encompassed herein. In specific embodiments, the immune cell is a NK cell, T cell, gamma delta T cell, invariant NKT (iNKT) cell, B cell, macrophage, MSC, or dendritic cell. In cases wherein the immune cell is an NK cell, the NK cell may be derived from cord blood, peripheral blood, induced pluripotent stem cells, bone marrow, or from a cell line. In specific aspects, the NK cell line is NK-92 cell line or another NK cell line derived from a tumor or from a healthy NK cell or a progenitor cell.

In specific embodiments, the immune cell is an NK cell, such as one derived from cord blood, such as from a cord blood mononuclear cell. The NK cell may be a CD56+NK cell, in specific cases. The NK cells may express one or more exogenously provided cytokines, such as IL-15, IL-2, IL-12, IL-18, IL-21, IL-7, or a combination thereof. Particular embodiments include populations of immune cells of any kind of the disclosure, and the cells may be present in a suitable medium or a suitable carrier of any kind.

In one embodiment, there is a method of killing CD70-positive cells in an individual, comprising the step of administering to the individual an effective amount of cells harboring any expression construct encompassed by the disclosure. In specific embodiments, the cells are NK cells, T cells, gamma delta T cells, invariant NKT (iNKT) cells, B cells, macrophages, gamma delta T cells, or dendritic cells. NK cells may be derived from cord blood, peripheral blood, induced pluripotent stem cells, bone marrow, or from a cell line. NK cells may be derived from cord blood mononuclear cells. In some cases, the CD70-positive cells are not cancer cells, although in other cases they are cancer cells. The CD70-positive cells may be T regulatory cells. In particular embodiments, the individual has acute myeloid leukemia, lymphoma, lung cancer, renal cancer, bladder cancer, melanoma, glioblastoma, breast cancer, head and neck cancer, mesothelioma, or a combination thereof. The cells may be allogeneic or autologous with respect to the individual, who may or may not be a human. The cells may be administered to the individual by injection, intravenously, intraarterially, intraperitoneally, intratracheally, intratumorally, intramuscularly, endoscopically, intralesionally, intracranially, percutaneously, subcutaneously, regionally, by perfusion, in a tumor microenvironment, or a combination thereof.

In particular embodiments of the methods, the cells may be administered to the individual once or more than once. The duration of time between administrations of the cells to the individual may be 1-24 hours, 1-7 days, 1-4 weeks, 1-12 months, or 1 or more years. The methods may further comprise the step of providing to the individual an effective amount of an additional therapy, such as surgery, radiation, gene therapy, immunotherapy, and/or hormone therapy. The additional therapy may comprise one or more antibodies or antibody-based agents, in some cases. In some aspects to the methods, they may further comprising the step of identifying CD70-positive cells in the individual.

In specific embodiments there is a composition of matter that comprises the sequences of SEQ ID NO:1, SEQ ID NO: 2, SEQ ID NO:7, SEQ ID NO:8, SEQ ID NO:9, SEQ ID NO:10, SEQ ID NO:11, SEQ ID NO:12, and SEQ ID NO:13.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Brief Summary, Detailed Description, Claims, and Brief Description of the Drawings.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIG. 1 shows an example of a plasmid map for the following codon optimized CD70 CAR vector: CO CAR.CD70 42D12. VLVH.IgG1.CD28.CD3z-2A-IL15.

FIG. 2 provides an example of a plasmid vector map for the CO CAR.CD70 42D12 VHVL.IgG1.CD28.CD3z-2A-IL15 vector.

FIG. 3 illustrates a plasmid vector map for CAR.CD70 42D12 VLVH.IgG1.CD28.CD3z-2A-IL15.

FIG. 4 provides a map of CAR.CD70 42D12 VHVL.IgG1.CD28.CD3z-2A-IL15.

FIG. 5A shows efficient transduction CD70 CARs in NK cells. The y-axes from top to bottom reads 10⁵, 10⁴, 10³, 0, and −10³, and the x-axes reads from left to right −10³, 0, 10³, 10⁴, and 10⁵. FIG. 5B shows expression of CD70 antigen on multiple acute myeloid leukemia (AML) cell lines.

FIG. 6 provides a functional assay demonstrating superior anti-tumor effector function of CAR.CD70/IL15 transduced NK cells. NK cells were expanded and were either non-transduced (NT) or transduced with CAR CD70/IL15 and their in vitro activity was tested against two AML cell lines (MOLM13 and MOLM14). CAR CD70/IL15 transduced NK cells secrete more IFN-g, TNFa and CD107a degranulation in response to targets compared to NT NK cells. The y-axes from top to bottom reads 10⁵, 10⁴, 10³, 0, and −10³, and the x-axes reads from left to right −10³, 0, 10³, 10⁴, and 10⁵.

FIG. 7 demonstrates greater killing of AML targets by CAR.CD70 NK cells as assessed by Annexin V assay. NK cells were expanded and were either non-transduced (NT) or transduced with CAR CD70/IL15 and their in vitro killing activity was tested against three AML cell lines (THP-1, MOLM14 and MOLM13). CAR CD70/IL15 transduced NK cells killed a greater proportion of leukemia targets as measured by live/dead and annexin V staining compared to NT NK cells. The y-axes from top to bottom reads 10⁵, 10⁴, 10³, 0, and −10³, and the x-axes reads from left to right −10³, 0, 10³, 10⁴, and 10⁵.

FIG. 8 shows greater killing of AML targets by CAR.CD70 NK cells, as assessed by Chromium release assay. NK cells were expanded and were either non-transduced (NT) or transduced with CAR CD70/IL15 and their in vitro killinh activity was tested against two AML cell lines (THP-1 and MOLM13). CAR CD70/IL15 transduced NK cells killed a greater proportion of leukemia targets as measured by 51 chromium relase assay compared to NT NK cells.

FIG. 9 demonstrates CD70 expression on a variety of lung cancer cell lines. The x-axes reads from left to right −10³, 0, 10³, 10⁴, and 10⁵.

FIGS. 10A-10B establish that compared to non-transduced (NT) and IL15-transduced NK cells, CAR.70 NK cells exert greater cytotoxicity against lung cancer. NK cells were expanded and were either non-transduced (NT) or transduced with IL15 (IL15) or with CAR CD70/IL15 (CD70 CAR) and their in vitro activity was tested against different lung cancer cell lines. CAR CD70/IL15 transduced NK cells secrete more IFN-g, TNFa and CD107a degranulation in response to targets compared to IL-15 transduced or NT NK cells. The y-axes from top to bottom reads 10⁵, 10⁴, 10³, 0, and −10³, and the x-axes reads from left to right −10³, 0, 10³, 10⁴, and 10⁵.

FIG. 11 shows that compared to non-transduced (NT) and IL15-transduced NK cells, CAR.70 NK cells exert greater cytotoxicity against lung cancer as assessed by annexin V staining. NK cells were expanded and were either non-transduced (NT) or transduced with CAR CD70/IL15 and their in vitro killing activity was tested against lung cancer cell lines. CAR CD70/IL15 transduced NK cells killed a greater proportion of lung cancer targets as measured by live/dead and annexin V staining compared to NT NK cells or IL15 NK cells. The y-axes from top to bottom reads 10⁵, 10⁴, 10³, 0, and −10³, and the x-axes reads from left to right −10³, 0, 10³, 10⁴, and 10⁵.

FIG. 12 demonstrates that compared to non-transduced (NT) and IL15-transduced NK cells, CAR.70 NK cells exert greater cytotoxicity against lung cancer cell lines, as assessed by caspase expression (green in a color version) in lung cancer cell line spheroids.

FIG. 13 establishes that compared to non-transduced (NT) and IL15-transduced NK cells, CAR.70 NK cells exert greater cytotoxicity against lung cancer cell line as assessed by measurement of green signal (caspase, green in a color version) using an Incucyte® assay.

FIG. 14 provides that compared to non-transduced (NT) and IL15-transduced NK cells, CAR.70 NK cells exert greater cytotoxicity against lung cancer as assessed by measurement of green signal (caspase, in a color version) using an Incucyte® assay.

FIG. 15 shows The Cancer Genome Atlas (TCGA) data regarding expression of CD70 on tumor cells.

FIGS. 16A-16B show CD70 CAR transduction efficiency in CBNK cells and expression of CD70 in various AML targets. (FIG. 16A) CD70 CAR was successfully transduced in CBNK cells with transduction efficiency of 98% when compared to non-transduced cells. The y-axes from top to bottom reads 10⁵, 10⁴, 10³, 0, and −10³, and the x-axes reads from left to right −10³, 0, 10³, 10⁴, and 10⁵. (FIG. 16B) CD70 was expressed in surface of various AML targets. The x-axes reads from left to right −10³, 0, 10³, 10⁴, and 10⁵.

FIG. 17 shows intracellular cytokines and degranulation marker expression in CBNK CD70 CAR cells when co-cultured with Molm13 and Molm14 cells. Comparison of non-transduced (NT) cells and CBNK cells transduced with CD70 CAR with respect to interferon gamma and tumor necrosis factor alpha secretion and degranulation marker CD107a expression when co-cultured with Molm13 (left) and Molm14 (right.) The y-axes from top to bottom reads 10⁵, 10⁴, 10³, 0, and −10³, and the x-axes reads from left to right −10³, 0, 10³, 10⁴, and 10⁵.

FIG. 18 shows Annexin V staining to assess the apoptosis of AML target cells after co-culture with CBNK CD70 CAR cells. Annexin V-LIVE/DEAD™ Fixable Aqua staining assay shows a comparison of non-transduced (NT) cells and CBNK cells transduced with CD70 CAR for THP-1, Molm13 and Molm14 cells. The y-axes from top to bottom reads 10⁵, 10⁴, 10³, 0, and −10³, and the x-axes reads from left to right −10³, 0, 10³, 10⁴, and 10⁵.

FIG. 19 demonstrates a chromium release assay to assess the cytotoxic activity of CBNK CD70 CAR against AML target cells. A comparison is provided for non-transduced (NT) cells and CBNK cells transduced with CD70 CAR with respect to cytotoxicity levels for THP-1 (left) and Molm13 (right) cells, as shown by chromium release assay.

FIGS. 20A-20B show an IncuCyte® cytotoxicity assay on THP-1 and OCI-AML3 cells when cocultured with CBNK CD70 CAR cells. A comparison of non-transduced (NT) cells and CBNK cells transduced with CD70 CAR for cytotoxicity is demonstrated for THP-1 (FIG. 20A) and OCI-AML3 (FIG. 20B) cells, as shown by IncuCyte® assay. CBNK cells transduced with IL15 construct was also used as a control in this assay.

FIG. 21 shows expression of CD70 in various lung cancer cell lines. Surface expression of CD70 was detected in various lung cancer cell lines using flow cytometry. The x-axes reads from left to right −10³, 0, 10³, 10⁴, and 10⁵.

FIG. 22 shows intracellular cytokines and degranulation marker expression in CBNK CD70 CAR cells when co-cultured with various lung cancer cell lines. Comparisons of non-transduced (NT) cells with CBNK cells transduced with CD70 CAR for levels of interferon gamma and tumor necrosis factor alpha secretion and degranulation marker CD107a expression are shown when co-cultured with various lung cancer cell lines. The y-axes from top to bottom reads 10⁵, 10⁴, 10³, 0, and −10³, and the x-axes reads from left to right −10³, 0, 10³, 10⁴, and 10⁵.

FIG. 23 demonstrates Annexin V staining to assess the apoptosis of lung cancer cells after co-culture with CBNK CD70 CAR cells. Comparison of apoptosis levels of non-transduced (NT) cells and CBNK cells transduced with CD70 CAR, as shown by Annexin V-LIVE/DEAD™ Fixable Aqua staining assay. The y-axes from top to bottom reads 10⁵, 10⁴, 10³, 0, and −10³, and the x-axes reads from left to right −10³, 0, 10³, 10⁴, and 10⁵.

FIG. 24 demonstrates an IncuCyte® cytotoxicity assay on ER1 cells when cocultured with CBNK CD70 CAR cells. Quantification of IncuCyte® cytotoxicity assay for 54 hours is shown in left panel, and representative images is shown in right panel.

FIG. 25 shows IncuCyte® cytotoxicity assay on ER3 cells when cocultured with CBNK CD70 CAR cells.

FIG. 26 shows a chromium release assay to assess the cytotoxic activity of CBNK CD70 CAR against breast cancer cell lines with varying CD70 expression. (Left) Surface expression of CD70 was detected in various breast cancer cell lines using flow cytometry (when two peaks, IgG is to the left). (Right) Comparison of cytotoxicity for non-transduced (NT) cells and CBNK cells transduced with CD70 CAR for BT549 and BCX010 cells, as shown by chromium release assay. K562 cells which are sensitive to NK cells are used as positive control. n.s. non significant; ***, P<0.001

FIGS. 27A-27E show intracellular cytokines and degranulation marker expression in CBNK CD70 CAR cells when co-cultured with various breast cancer cells. (FIG. 27A) No cells; (FIG. 27B) K562 cells; (FIG. 27C) MDA-MB-231 cells; (FIG. 27D) BT549; (FIG. 27E) BCX010 cells. n.s. non significant; *, p<0.05; **, p<0.01; ***, p<0.001. From left to right, the groupings of three for the bars is CBNK NT, CBNK IL15, and CBNK CAR CD70.

FIGS. 28A-28B demonstrate a chromium release assay to assess the cytotoxic activity of CBNK CD70 CAR against multiple myeloma. (FIG. 28A) Surface expression of CD70 is shown for MM1s, a multiple myeloma cell line, as detected by using flow cytometry. (FIG. 28B) Comparison of cytotoxicity for non-transduced (NT) cells and CBNK cells transduced with CD70 CAR, as shown by chromium release assay.

FIGS. 29A-29B show a chromium release assay to assess the cytotoxic activity of CBNK CD70 CAR against renal cell carcinoma. (FIG. 29A) Detection of surface expression of CD70 in various RCC and other cancer cell lines using flow cytometry. A498, SN12C and 786-0 are RCC cell lines with high CD70 expression. (FIG. 29B) Levels of cytotoxicity are compared for non-transduced (NT) cells and CBNK cells transduced with CD70 CAR, as shown by chromium release assay.

FIG. 30 shows intracellular cytokines and degranulation marker expression in CBNK CD70 CAR cells when co-cultured with RCC cells. **, p<0.01

FIG. 31 shows IncuCyte® cytotoxicity assay on 786-0 RCC cells when cocultured with CBNK CD70 CAR cells, as assessed by the measurement of green (caspase 3/7) signal. **, p<0.01; ***, p<0.001

FIGS. 32A-32B show intracellular cytokines expression in CBNK CD70 CAR cells when co-cultured with pancreatic cancer cells. (FIG. 32A) Surface expression of CD70 was measured in various pancreatic cancer cell lines using flow cytometry. The y-axes from top to bottom reads 250K, 200K, 150K, 100K, 50K, and 0, and the x-axes reads from left to right 0, 10³, 10⁴, and 10⁵. (FIG. 32B) Comparison of interferon gamma and tumor necrosis factor alpha secretion for non-transduced (NT) cells and CBNK cells transduced with CD70 CAR when co-cultured with PANC-1 cell line or MIA-Paca2 cell line (low CD70 expression). MFI represents Mean Fluorescence Intensity (a representative of the level of expression).

FIG. 33 demonstrates an IncuCyte® cytotoxicity assay on GSC20 glioblastoma cells when cocultured with CBNK CD70 CAR cells. (i) Surface expression of CD70 was detected in various GBM cell lines using flow cytometry and GSC20 cell line showed the highest CD70 surface expression. Compared to non-transduced (NT) cells, CBNK cells transduced with CD70 CAR showed increased cytotoxicity of GSC20 cells, as shown by IncuCyte® assay, as assessed by the measurement of green (caspase 3/7) signal intensity, suggesting that CBNK CD70 CAR cells have greater killing activity against GBM cells. Quantification of IncuCyte® cytotoxicity assay for 57 hours is shown in ii, and representative images up to 23 hours is shown in iii.

FIG. 34 shows survival curve of NOD scid gamma mouse (NSG mice that are immunodeficient) engrafted with either Raji WT or CD70 KO cells and treated with CBNK CD70 CAR cells. *p<0.05

While various embodiments of the disclosure have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the disclosure described herein may be employed.

### DETAILED DESCRIPTION

### 1. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure.

The term “isolated” as used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, such as that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides that are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

As used herein, “prevent,” and similar words such as “prevented,” “preventing” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

The term “sample,” as used herein, generally refers to a biological sample. The sample may be taken from tissue or cells from an individual. In some examples, the sample may comprise, or be derived from, a tissue biopsy, blood (e.g., whole blood), blood plasma, extracellular fluid, dried blood spots, cultured cells, discarded tissue. The sample may have been isolated from the source prior to collection. Non-limiting examples include blood, cerebral spinal fluid, pleural fluid, amniotic fluid, lymph fluid, saliva, urine, stool, tears, sweat, or mucosal excretions, and other bodily fluids isolated from the primary source prior to collection. In some examples, the sample is isolated from its primary source (cells, tissue, bodily fluids such as blood, environmental samples, etc.) during sample preparation. The sample may or may not be purified or otherwise enriched from its primary source. In some cases the primary source is homogenized prior to further processing. The sample may be filtered or centrifuged to remove buffy coat, lipids, or particulate matter. The sample may also be purified or enriched for nucleic acids, or may be treated with RNases. The sample may contain tissues or cells that are intact, fragmented, or partially degraded.

The term “subject,” as used herein, generally refers to an individual having a biological sample that is undergoing processing or analysis and, in specific cases, has or is suspected of having cancer. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as benign or malignant neoplasias, or cancer. The subject may being undergoing or having undergone treatment. The subject may be asymptomatic. The subject may be healthy individuals but that are desirous of prevention of cancer. The term “individual” may be used interchangeably, in at least some cases. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

The present disclosure concerns methods and compositions directed to genetically engineered mammalian immune cells of any kind (including at least human NK cells) to target CD70-positive tumors. The disclosure encompasses a genetically engineered receptor of any kind (including a CAR) that is directed against CD70, the ligand for the cytokine receptor CD27. CD70 is an attractive ‘pan-cancer antigen,’ because in addition to being expressed on hematologic malignancies, such as acute myeloid leukemia (AML) and lymphoma, it also expressed on many solid tumors, and cancers include renal, bladder, lung, breast, glioblastoma, pancreatic, and melanoma. It is only transiently found on activated T and B lymphocytes and dendritic cells. CD70 is particularly advantageous as a target for the immunotherapy of AML, because, unlike other AML targets, it is not expressed on normal hematopoietic stem cells and therefore is unlikely to result in prolonged cytopenias and the need for hematopoietic stem cell transplant for the recipient after CAR therapy. In specific embodiments there are provided a number of novel expression constructs, including retroviral constructs, that express a single chain variable fragment (scFv) against CD70 in a CAR and also expresses one or more cytokines, such as IL-15, to support NK cell survival and proliferation. In a series of in vitro studies provided herein, the activity of CAR70/IL-15 transduced cord blood (CB)-NK cells against AML, lung cancer targets and glioblastoma is demonstrated.

### I. GENETICALLY ENGINEERED RECEPTORS

The immune cells of the present disclosure can be genetically engineered to express antigen receptors that target CD70, such as engineered TCRs or CARs. For example, the immune cells may be NK cells that are modified to express a CAR and/or TCR having antigenic specificity for CD70. Other CARs and/or TCRs may be expressed by the same cells as the CD70 receptor-expressing cells, and they may be directed to different antigens. In some aspects, the immune cells are engineered to express the CD70-specific CAR or CD70-specific TCR by knock-in of the CAR or TCR using CRISPR.

Suitable methods of modification are known in the art. See, for instance, Sambrook and Ausubel, supra. For example, the cells may be transduced to express a TCR having antigenic specificity for a cancer antigen using transduction techniques described in Heemskerk et al., 2008 and Johnson et al., 2009.

In some embodiments, the cells comprise one or more nucleic acids introduced via genetic engineering that encode one or more antigen receptors (at least one of which is directed against CD70), and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric).

Exemplary antigen receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., 2013; Davila et al., 2013; Turtle et al., 2012; Wu et al., 2012. In some aspects, the genetically engineered antigen receptors include a CAR as described in U.S. Pat. No. 7,446,190, and those described in International Patent Application Publication No.: WO/2014055668 Al.

A. Chimeric Antigen Receptors

In some embodiments, the CD70-specific CAR comprises: a) one or more intracellular signaling domains, b) a transmembrane domain, and c) an extracellular domain comprising an antigen binding region that targets, including specifically binds, CD70. In particular embodiments the antigen binding region is an antibody and is not a protein or protein fragment that is not an antibody.

In some embodiments, the engineered antigen receptors include CARs, including activating or stimulatory CARs, costimulatory CARs (see WO2014/055668), and/or inhibitory CARs (iCARs, see Fedorov et al., 2013). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

Certain embodiments of the present disclosure concern the use of nucleic acids, including nucleic acids encoding an CD70-specific CAR polypeptide, including a CAR that has been humanized to reduce immunogenicity (hCAR), comprising at least one intracellular signaling domain, a transmembrane domain, and an extracellular domain comprising one or more signaling motifs. In certain embodiments, the CD70-specific CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the human CD70 CAR nucleic acids may be human genes used to enhance cellular immunotherapy for human patients. In a specific embodiment, the disclosure includes a full-length CD70-specific CAR cDNA or coding region. The antigen binding regions or domain can comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is a CD70-specific scFv encoded by a sequence that is optimized for human codon usage for expression in human cells.

The arrangement could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin. One could also use portions of CD8alpha.

In some embodiments, the CD70 CAR nucleic acid comprises a sequence encoding other costimulatory receptors, such as a transmembrane domain and a modified CD28 intracellular signaling domain. Other costimulatory receptors include, but are not limited to one or more of CD28, CD27, OX-40 (CD134), DAP10, DAP12, and 4-1BB (CD137). In addition to a primary signal initiated by CD3, an additional signal provided by a human costimulatory receptor inserted in a human CAR is important for full activation of NK cells and could help improve in vivo persistence and the therapeutic success of the adoptive immunotherapy.

In some embodiments, CD70-specific CAR is constructed with specificity for CD70, such as CD70 being expressed on a normal or non-diseased cell type or on a diseased cell type Thus, the CAR typically includes in its extracellular portion one or more CD70 binding molecules, such as one or more antigen-binding fragment, domain, or portion, or one or more antibody variable domains, and/or antibody molecules. In some embodiments, the CD70-specific CAR includes an antigen-binding portion or portions of an antibody molecule, such as a single-chain antibody fragment (scFv) derived from the variable heavy (VH) and variable light (VL) chains of a monoclonal antibody (mAb).

In certain embodiments, the CD70-specific CAR may be co-expressed with a cytokine to improve persistence when there is a low amount of tumor-associated antigen. For example, the CAR may be co-expressed with one or more cytokines, such as IL-7, IL-2, IL-15, IL-12, IL-18, IL-21, IL-7, or a combination thereof.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, and DAP molecules. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

In certain embodiments, the platform technologies disclosed herein to genetically modify immune cells, such as NK cells, comprise (i) non-viral gene transfer using an electroporation device (e.g., a nucleofector), (ii) CARs that signal through endodomains (e.g., CD28/CD3-ζ CD137/CD3-ζ or other combinations), (iii) CARs with variable lengths of extracellular domains connecting the CD70-recognition domain to the cell surface, and, in some cases, (iv) artificial antigen presenting cells (aAPC) derived from K562 to be able to robustly and numerically expand CARP immune cells (Singh et al., 2008; Singh et al., 2011).

B. Examples of Specific CAR Embodiments

In particular embodiments, specific CD70 CAR molecules, or vectors encoding multiple molecules including a CD70-specific CAR, are encompassed herein. In some cases, the CD70 binding domain of the CAR is a scFv, and any scFv that binds to CD70 may be utilized herein. The variable heavy chain and the variable light chain for the scFv may be in any order in N-terminal to C-terminal direction. For example, the variable heavy chain may be on the N-terminal side of the variable light chain, or vice versa. The scFv may or may not be codon optimized. The scFv may or may not be humanized. Specific examples of CD70 scFvs include at least [symbol] or any others. The scFv that is utilized may be at least 80, 81, 82, 83, 84, 85, 86, 87, 88, 89, 90, 91, 92, 93, 94, 95, 96, 97, 98, 99, or 100% identical to 42D12, Ab7, 27B3, 9D1, 57B6, or any others.

In particular embodiments, a vector encodes a CD70-specific CAR and also encodes one or more other molecules. For example, a vector may encode a CD70-specific CAR that may or may not be codon optimized (CO), and in specific cases the anti-CD70 scFv is the 42D12 scFv that may have the variable light chain upstream or down stream of the variable heavy chain. In specific embodiments, the CAR comprises CD28 and no other costimulatory domain, and the CAR may also comprise CD3. In some cases, the vector also encodes one or more cytokines and one or more suicide genes.

A DNA sequence and polypeptide sequence for codon optimized (CO) CAR.CD70 42D12 VLVH scFv antibody sequence is as follows:

DNA
(SEQ ID NO: 1)
ATGGCCCTGCCTGTGACAGCTCTGCTCCTCCCTCTGGCCCTGCTGCTCC
ATGCCGCCAGACCCCAGGCAGTtGTGACCCAGGAGCCTTCCCTGACAGT
GTCTCCAGGAGGGACGGTCACGCTCACCTGCGGCCTCAAATCTGGGTCT
GTCACTTCCGATAACTTCCCCACTTGGTACCAGCAGACACCAGGCCAGG
CTCCCCGATTGCTTATCTACAACACAAACACCCGTCACTCTGGCGTCCC
CGACCGCTTCTCCGGATCCATCCTGGGCAACAAAGCCGCCCTCACCATC
ACGGGGGCCCAGGCCGACGACGAGGCCGAATATTTCTGTGCTCTGTTCA
TAAGTAATCCTAGTGTTGAGTTCGGCGGAGGGACCCAACTGACCGTCCT
AGGTGGCAGCACCAGCGGCTCCGGCAAGCCTGGCTCTGGCGAGGGCAGC
ACAAAGGGAGAGGTGCAGCTCGTGGAGTCTGGGGGAGGCTTGGTGCAGC
CTGGGGGGTCTCTGAGACTCTCCTGTGCAGCCTCTGGATTCACCTTCAG
TGTCTACTACATGAACTGGGTCCGCCAGGCTCCAGGGAAGGGGCTCGAG
TGGGTCTCAGATATTAATAATGAAGGTGGTACTACATACTATGCAGACT
CCGTGAAGGGCCGATTCACCATCTCCAGAGACAACTCTAAGAACAGCCT
GTATCTGCAAATGAACAGCCTGCGCGCCGAGGACACGGCCGTGTACTAC
TGCGCGAGAGATGCCGGATATAGCAACCATGTACCCATCTTTGATTCTT
GGGGCCAGGGGACCCTGGTCACTGTCTCCTCA
Protein
(SEQ ID NO: 2)
MALPVTALLLPLALLLHAARPQAVVTQEPSLTVSPGGTVTLTCGLKSGS
VTSDNFPTWYQQTPGQAPRLLIYNTNTRHSGVPDRFSGSILGNKAALTI
TGAQADDEAEYFCALFISNPSVEFGGGTQLTVLGGSTSGSGKPGSGEGS
TKGEVQLVESGGGLVQPGGSLRLSCAASGFTFSVYYMNWVRQAPGKGLE
WVSDINNEGGTTYYADSVKGRFTISRDNSKNSLYLQMNSLRAEDTAVYY
CARDAGYSNHVPIFDSWGQGTLVTVSS

The DNA and protein sequence for the following scFv antibody sequence that is not codon optimized (CAR.CD70 42D12 VHVL sequence) is as follows:

DNA
(SEQ ID NO: 3)
ATGGCCCTGCCTGTGACAGCTCTGCTCCTCCCTCTGGCCCTGCTGCTCCA
TGCCGCCAGACCCGAGGTGCAGCTCGTGGAGTCTGGGGGAGGCTTGGTGC
AGCCTGGGGGGTCTCTGAGACTCTCCTGTGCAGCCTCTGGATTCACCTTC
AGTGTCTACTACATGAACTGGGTCCGCCAGGCTCCAGGGAAGGGGCTCGA
GTGGGTCTCAGATATTAATAATGAAGGTGGTACTACATACTATGCAGACT
CCGTGAAGGGCCGATTCACCATCTCCAGAGACAACTCTAAGAACAGCCTG
TATCTGCAAATGAACAGCCTGCGCGCCGAGGACACGGCCGTGTACTACTG
CGCGAGAGATGCCGGATATAGCAACCATGTACCCATCTTTGATTCTTGGG
GCCAGGGGACCCTGGTCACTGTCTCCTCAGGCAGCACCAGCGGCTCCGGC
AAGCCTGGCTCTGGCGAGGGCAGCACAAAGGGACAGGCAGTGGTGACCCA
GGAGCCTTCCCTGACAGTGTCTCCAGGAGGGACGGTCACGCTCACCTGCG
GCCTCAAATCTGGGTCTGTCACTTCCGATAACTTCCCCACTTGGTACCAG
CAGACACCAGGCCAGGCTCCCCGATTGCTTATCTACAACACAAACACCCG
TCACTCTGGCGTCCCCGACCGCTTCTCCGGATCCATCCTGGGCAACAAAG
CCGCCCTCACCATCACGGGGGCCCAGGCCGACGACGAGGCCGAATATTTC
TGTGCTCTGTTCATAAGTAATCCTAGTGTTGAGTTCGGCGGAGGGACCCA
ACTGACCGTCCTAGGT
Protein
(SEQ ID NO: 4)
MGMALPVTALLLPLALLLHAARPEVQLVESGGGLVQPGGSLRLSCAASGF
TFSVYYMNWVRQAPGKGLEWVSDINNEGGTTYYADSVKGRFTISRDNSKN
SLYLQMNSLRAEDTAVYYCARDAGYSNHVPIFDSWGQGTLVTVSSGSTSG
SGKPGSGEGSTKGQAVVTQEPSLTVSPGGTVTLTCGLKSGSVTSDNFPTW
YQQTPGQAPRLLIYNTNTRHSGVPDRFSGSILGNKAALTITGAQADDEAE
YFCALFISNPSVEFGGGTQLTVLG

The DNA and protein sequence for the following scFv antibody sequence CAR.CD70 42D12 VLVH is as follows:

DNA
(SEQ ID NO: 5)
ATGGCCCTGCCTGTGACAGCTCTGCTCCTCCCTCTGGCCCTGCTGCTCC
ATGCCGCCAGACCCGAGGTGCAGCTCGTGGAGTCTGGGGGAGGCTTGGT
GCAGCCTGGGGGGTCTCTGAGACTCTCCTGTGCAGCCTCTGGATTCACC
TTCAGTGTCTACTACATGAACTGGGTCCGCCAGGCTCCAGGGAAGGGGC
TTGAGTGGGTCTCAGATATTAATAATGAAGGTGGTACTACATACTATGC
AGACTCCGTGAAGGGCCGATTCACCATCTCCAGAGACAACTCTAAGAAC
AGCCTGTATCTGCAAATGAACAGCCTGCGCGCCGAGGACACGGCCGTGT
ACTACTGCGCGAGAGATGCCGGATATAGCAACCATGTACCCATCTTTGA
TTCTTGGGGCCAGGGGACCCTGGTCACTGTCTCCTCAGGCAGCACCAGC
GGCTCCGGCAAGCCTGGCTCTGGCGAGGGCAGCACAAAGGGACAGGCAG
TGGTGACCCAGGAGCCTTCCCTGACAGTGTCTCCAGGAGGGACGGTCAC
GCTCACCTGCGGCCTCAAATCTGGGTCTGTCACTTCCGATAACTTCCCC
ACTTGGTACCAGCAGACACCAGGCCAGGCTCCCCGATTGCTTATCTACA
ACACAAACACCCGTCACTCTGGCGTCCCCGACCGCTTCTCCGGATCCAT
CCTGGGCAACAAAGCCGCCCTCACCATCACGGGGGCCCAGGCCGACGAC
GAGGCCGAATATTTCTGTGCTCTGTTCATAAGTAATCCTAGTGTTGAGT
TCGGCGGAGGGACCCAACTGACCGTCCTAGGT
Protein
(SEQ ID NO: 6)
MGMALPVTALLLPLALLLHAARPEVQLVESGGGLVQPGGSLRLSCAASG
FTFSVYYMNWVRQAPGKGLEWVSDINNEGGTTYYADSVKGRFTISRDNS
KNSLYLQMNSLRAEDTAVYYCARDAGYSNHVPIFDSWGQGTLVTVSSGS
TSGSGKPGSGEGSTKGQAVVTQEPSLTVSPGGTVTLTCGLKSGSVTSDN
FPTWYQQTPGQAPRLLIYNTNTRHSGVPDRFSGSILGNKAALTITGAQA
DDEAEYFCALFISNPSVEFGGGTQLTVLG

Examples of specific vector molecules including a CAR and IL15 encompass at least the following:

CO CAR.CD70 42D12. VLVH.IgG1.CD28.CD3z-2A-IL15

CO CAR.CD70 42D12 VHVL.IgG1.CD28.CD3z-2A-IL15

CAR.CD70 42D12 VLVH.IgG1.CD28.CD3z-2A-IL15

CAR.CD70 42D12 VHVL.IgG1.CD28.CD3z-2A-IL15

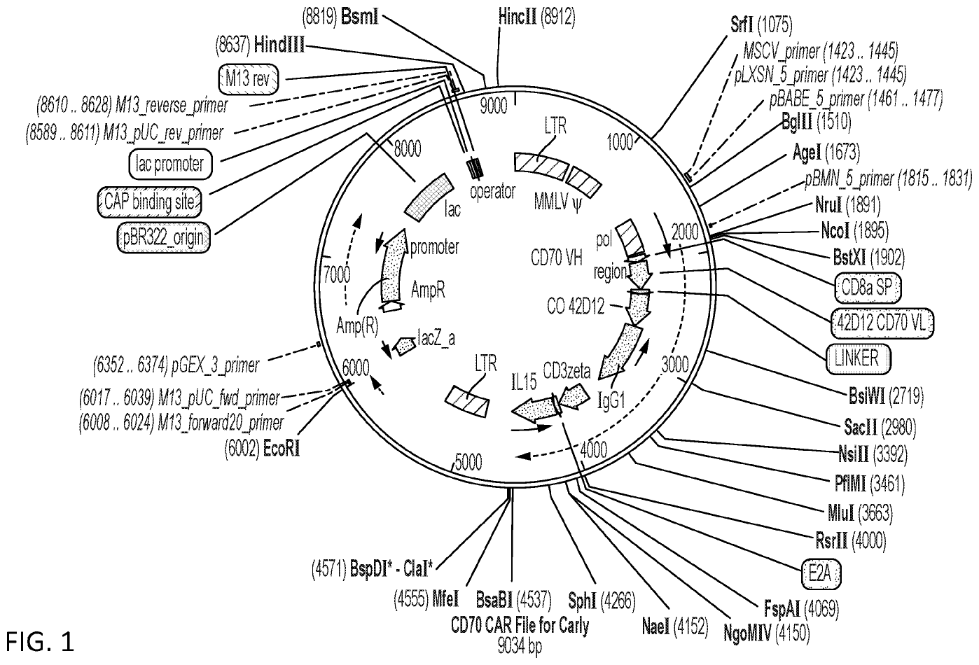

An example of a plasmid map for the exemplary CO CAR.CD70 42D12. VLVH.IgG1.CD28.CD3z-2A-IL15 vector is in FIG. 1. The full DNA sequence for the vector comprising CO CAR.CD70 42D12. VLVH.IgG1.CD28.CD3z-2A-IL15 is as follows:

(SEQ ID NO: 7)
ATGGCCCTGCCTGTGACAGCTCTGCTCCTCCCTCTGGCCCTGCTGCTCCATGC
CGCCAGACCCCAGGCAGTtGTGACCCAGGAGCCTTCCCTGACAGTGTCTCCAGGAGG
GACGGTCACGCTCACCTGCGGCCTCAAATCTGGGTCTGTCACTTCCGATAACTTCCC
CACTTGGTACCAGCAGACACCAGGCCAGGCTCCCCGATTGCTTATCTACAACACAAA
CACCCGTCACTCTGGCGTCCCCGACCGCTTCTCCGGATCCATCCTGGGCAACAAAGC
CGCCCTCACCATCACGGGGGCCCAGGCCGACGACGAGGCCGAATATTTCTGTGCTCT
GTTCATAAGTAATCCTAGTGTTGAGTTCGGCGGAGGGACCCAACTGACCGTCCTAGG
TGGCAGCACCAGCGGCTCCGGCAAGCCTGGCTCTGGCGAGGGCAGCACAAAGGGAG
AGGTGCAGCTCGTGGAGTCTGGGGGAGGCTTGGTGCAGCCTGGGGGGTCTCTGAGA
CTCTCCTGTGCAGCCTCTGGATTCACCTTCAGTGTCTACTACATGAACTGGGTCCGCC
AGGCTCCAGGGAAGGGGCTCGAGTGGGTCTCAGATATTAATAATGAAGGTGGTACT
ACATACTATGCAGACTCCGTGAAGGGCCGATTCACCATCTCCAGAGACAACTCTAA
GAACAGCCTGTATCTGCAAATGAACAGCCTGCGCGCCGAGGACACGGCCGTGTACT
ACTGCGCGAGAGATGCCGGATATAGCAACCATGTACCCATCTTTGATTCTTGGGGCC
AGGGGACCCTGGTCACTGTCTCCTCACGTACGGTCACTGTCTCTTCACAGGATCCCG
CCGAGCCCAAATCTCCTGACAAAACTCACACATGCCCACCGTGCCCAGCACCTGAA
CTCCTGGGGGGACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACCC
TGAGGTCAAGTTCAACTGGTACGTGGACGGCGTGGAGGTGCATAATGCCAAGACAA
AGCCGCGGGAGGAGCAGTACAACAGCACGTACCGTGTGGTCAGCGTCCTCACCGTC
CTGCACCAGGACTGGCTGAATGGCAAGGAGTACAAGTGCAAGGTCTCCAACAAAGC
CCTCCCAGCCCCCATCGAGAAAACCATCTCCAAAGCCAAAGGGCAGCCCCGAGAAC
CACAGGTGTACACCCTGCCCCCATCCCGGGATGAGCTGACCAAGAACCAGGTCAGC
CTGACCTGCCTGGTCAAAGGCTTCTATCCCAGCGACATCGCCGTGGAGTGGGAGAG
CAATGGGCAACCGGAGAACAACTACAAGACCACGCCTCCCGTGCTGGACTCCGACG
GCTCCTTCTTCCTCTACAGCAAGCTCACCGTGGACAAGAGCAGGTGGCAGCAGGGG
AACGTCTTCTCATGCTCCGTGATGCATGAGGCTCTGCACAACCACTACACGCAGAAG
AGCCTCTCCCTGTCTCCGGGTAAAAAAGATCCCAAATTTTGGGTGCTGGTGGTGGTT
GGTGGAGTCCTGGCTTGCTATAGCTTGCTAGTAACAGTGGCCTTTATTATTTTCTGGG
TGAGGAGTAAGAGGAGCAGGCTCCTGCACAGTGACTACATGAACATGACTCCCCGC
CGCCCCGGGCCCACCCGCAAGCATTACCAGCCCTATGCCCCACCACGCGACTTCGCA
GCCTATCGCTCACGCGTGAAGTTCAGCAGGAGCGCAGACGCCCCCGCGTACCAGCA
GGGCCAGAACCAGCTCTATAACGAGCTCAATCTAGGACGAAGAGAGGAGTACGATG
TTTTGGACAAAAGACGTGGCCGGGACCCTGAGATGGGGGGAAAGCCGAGAAGGAA
GAACCCTCAGGAAGGCCTGTACAATGAACTGCAGAAAGATAAGATGGCGGAGGCCT
ACAGTGAGATTGGGATGAAAGGCGAGCGCCGGAGGGGCAAGGGGCACGATGGCCT
TTACCAGGGTCTCAGTACAGCCACCAAGGACACCTACGACGCCCTTCACATGCAGG
CCCTGCCCCCTCGCGGACCGCAGTGTACTAATTATGCTCTCTTGAAATTGGCTGGAG
ATGTTGAGAGCAATCCCGGGCCCATGCGCATTAGCAAGCCCCACCTGCGGAGCATC
AGCATCCAGTGCTACCTGTGCCTGCTGCTGAACAGCCACTTCCTGACCGAGGCCGGC
ATCCACGTGTTCATCCTGGGCTGCTTCAGCGCCGGACTGCCCAAGACCGAGGCCAAC
TGGGTGAACGTGATCAGCGACCTGAAGAAGATCGAGGACCTGATCCAGAGCATGCA
CATCGACGCCACCCTGTACACCGAGAGCGACGTGCACCCCAGCTGCAAGGTGACCG
CCATGAAGTGCTTTCTGCTGGAACTGCAGGTGATCAGCCTGGAAAGCGGCGACGCC
AGCATCCACGACACCGTGGAGAACCTGATCATCCTGGCCAACAACAGCCTGAGCAG
CAACGGCAACGTGACCGAGAGCGGCTGCAAAGAGTGCGAGGAACTGGAAGAGAAG
AACATCAAAGAGTTTCTGCAGAGCTTCGTGCACATCGTGCAGATGTTCATCAACACC
AGCTGA

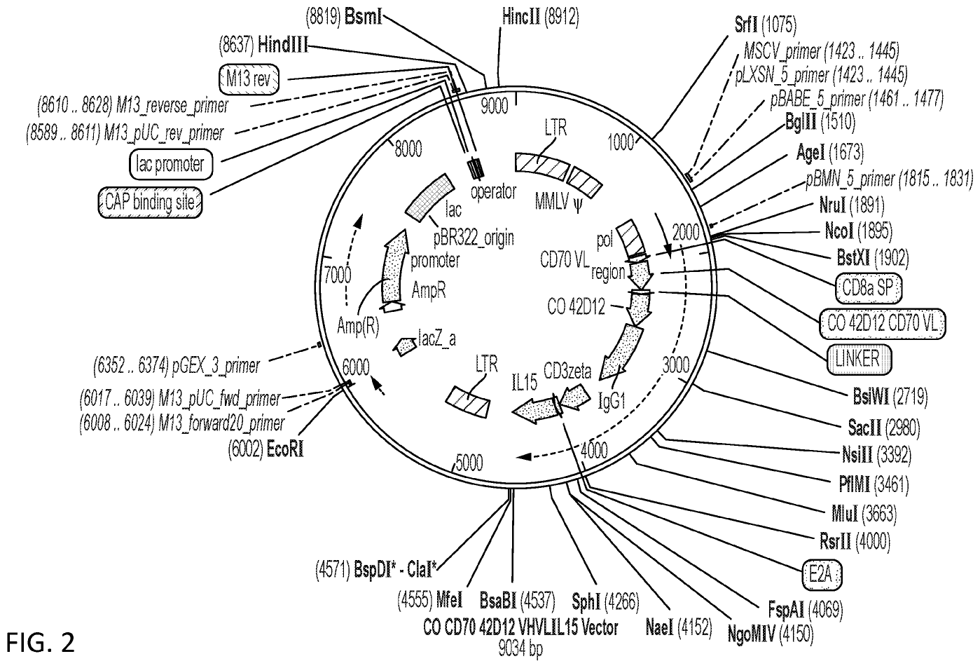

In some embodiments, a codon optimized CO CAR.CD70 42D12 VHVL.IgG1.CD28.CD3z-2A-IL15 vector is employed. An example of a plasmid map for a codon optimized CO CAR.CD70 42D12 VHVL.IgG1.CD28.CD3z-2A-IL15 vector is in FIG. 2. A full DNA sequence for the following construct CO CAR.CD70 42D12 VHVL.IgG1.CD28.CD3z-2A-IL15 is as follows:

(SEQ ID NO: 8)
ATGGCCCTGCCTGTGACAGCTCTGCTCCTCCCTCTGGCCCTGCTGCTCCATGC
CGCCAGACCCGAGGTGCAGCTCGTGGAGTCTGGGGGAGGCTTGGTGCAGCCTGGGG
GGTCTCTGAGACTCTCCTGTGCAGCCTCTGGATTCACCTTCAGTGTCTACTACATGAA
CTGGGTCCGCCAGGCTCCAGGGAAGGGGCTCGAGTGGGTCTCAGATATTAATAATG
AAGGTGGTACTACATACTATGCAGACTCCGTGAAGGGCCGATTCACCATCTCCAGAG
ACAACTCTAAGAACAGCCTGTATCTGCAAATGAACAGCCTGCGCGCCGAGGACACG
GCCGTGTACTACTGCGCGAGAGATGCCGGATATAGCAACCATGTACCCATCTTTGAT
TCTTGGGGCCAGGGGACCCTGGTCACTGTCTCCTCAGGCAGCACCAGCGGCTCCGGC
AAGCCTGGCTCTGGCGAGGGCAGCACAAAGGGACAGGCAGTGGTGACCCAGGAGC
CTTCCCTGACAGTGTCTCCAGGAGGGACGGTCACGCTCACCTGCGGCCTCAAATCTG
GGTCTGTCACTTCCGATAACTTCCCCACTTGGTACCAGCAGACACCAGGCCAGGCTC
CCCGATTGCTTATCTACAACACAAACACCCGTCACTCTGGCGTCCCCGACCGCTTCT
CCGGATCCATCCTGGGCAACAAAGCCGCCCTCACCATCACGGGGGCCCAGGCCGAC
GACGAGGCCGAATATTTCTGTGCTCTGTTCATAAGTAATCCTAGTGTTGAGTTCGGC
GGAGGGACCCAACTGACCGTCCTAGGTCGTACGGTCACTGTCTCTTCACAGGATCCC
GCCGAGCCCAAATCTCCTGACAAAACTCACACATGCCCACCGTGCCCAGCACCTGA
ACTCCTGGGGGGACCGTCAGTCTTCCTCTTCCCCCCAAAACCCAAGGACACCCTCAT
GATCTCCCGGACCCCTGAGGTCACATGCGTGGTGGTGGACGTGAGCCACGAAGACC
CTGAGGTCAAGTTCAACTGGTACGTGGACGGCGTGGAGGTGCATAATGCCAAGACA
AAGCCGCGGGAGGAGCAGTACAACAGCACGTACCGTGTGGTCAGCGTCCTCACCGT
CCTGCACCAGGACTGGCTGAATGGCAAGGAGTACAAGTGCAAGGTCTCCAACAAAG
CCCTCCCAGCCCCCATCGAGAAAACCATCTCCAAAGCCAAAGGGCAGCCCCGAGAA
CCACAGGTGTACACCCTGCCCCCATCCCGGGATGAGCTGACCAAGAACCAGGTCAG
CCTGACCTGCCTGGTCAAAGGCTTCTATCCCAGCGACATCGCCGTGGAGTGGGAGA
GCAATGGGCAACCGGAGAACAACTACAAGACCACGCCTCCCGTGCTGGACTCCGAC
GGCTCCTTCTTCCTCTACAGCAAGCTCACCGTGGACAAGAGCAGGTGGCAGCAGGG
GAACGTCTTCTCATGCTCCGTGATGCATGAGGCTCTGCACAACCACTACACGCAGAA
GAGCCTCTCCCTGTCTCCGGGTAAAAAAGATCCCAAATTTTGGGTGCTGGTGGTGGT
TGGTGGAGTCCTGGCTTGCTATAGCTTGCTAGTAACAGTGGCCTTTATTATTTTCTGG
GTGAGGAGTAAGAGGAGCAGGCTCCTGCACAGTGACTACATGAACATGACTCCCCG
CCGCCCCGGGCCCACCCGCAAGCATTACCAGCCCTATGCCCCACCACGCGACTTCGC
AGCCTATCGCTCACGCGTGAAGTTCAGCAGGAGCGCAGACGCCCCCGCGTACCAGC
AGGGCCAGAACCAGCTCTATAACGAGCTCAATCTAGGACGAAGAGAGGAGTACGAT
GTTTTGGACAAAAGACGTGGCCGGGACCCTGAGATGGGGGGAAAGCCGAGAAGGA
AGAACCCTCAGGAAGGCCTGTACAATGAACTGCAGAAAGATAAGATGGCGGAGGCC
TACAGTGAGATTGGGATGAAAGGCGAGCGCCGGAGGGGCAAGGGGCACGATGGCC
TTTACCAGGGTCTCAGTACAGCCACCAAGGACACCTACGACGCCCTTCACATGCAGG
CCCTGCCCCCTCGCGGACCGCAGTGTACTAATTATGCTCTCTTGAAATTGGCTGGAG
ATGTTGAGAGCAATCCCGGGCCCATGCGCATTAGCAAGCCCCACCTGCGGAGCATC
AGCATCCAGTGCTACCTGTGCCTGCTGCTGAACAGCCACTTCCTGACCGAGGCCGGC
ATCCACGTGTTCATCCTGGGCTGCTTCAGCGCCGGACTGCCCAAGACCGAGGCCAAC
TGGGTGAACGTGATCAGCGACCTGAAGAAGATCGAGGACCTGATCCAGAGCATGCA
CATCGACGCCACCCTGTACACCGAGAGCGACGTGCACCCCAGCTGCAAGGTGACCG
CCATGAAGTGCTTTCTGCTGGAACTGCAGGTGATCAGCCTGGAAAGCGGCGACGCC
AGCATCCACGACACCGTGGAGAACCTGATCATCCTGGCCAACAACAGCCTGAGCAG
CAACGGCAACGTGACCGAGAGCGGCTGCAAAGAGTGCGAGGAACTGGAAGAGAAG
AACATCAAAGAGTTTCTGCAGAGCTTCGTGCACATCGTGCAGATGTTCATCAACACC
AGCTGA

Non codon-optimized CARs may also be employed, such as a CAR.CD70 42D12 VLVH.IgG1.CD28.CD3z-2A-IL15 Vector, and a sequence is provided below:

(SEQ ID NO: 9)
ATGGCCCTGCCTGTGACAGCTCTGCTCCTCCCTCTGGCCCTGCTGCTCCATGC
CGCCAGACCCCAGGCAGTtGTGACCCAGGAGCCTTCCCTGACAGTGTCTCCAGGAGG
GACGGTCACGCTCACCTGCGGCCTCAAATCTGGGTCTGTCACTTCCGATAACTTCCC
CACTTGGTACCAGCAGACACCAGGCCAGGCTCCCCGATTGCTTATCTACAACACAAA
CACCCGTCACTCTGGCGTCCCCGACCGCTTCTCCGGATCCATCCTGGGCAACAAAGC
CGCCCTCACCATCACGGGGGCCCAGGCCGACGACGAGGCCGAATATTTCTGTGCTCT
GTTCATAAGTAATCCTAGTGTTGAGTTCGGCGGAGGGACCCAACTGACCGTCCTAGG
TGGCAGCACCAGCGGCTCCGGCAAGCCTGGCTCTGGCGAGGGCAGCACAAAGGGAG
AGGTGCAGCTCGTGGAGTCTGGGGGAGGCTTGGTGCAGCCTGGGGGGTCTCTGAGA
CTCTCCTGTGCAGCCTCTGGATTCACCTTCAGTGTCTACTACATGAACTGGGTCCGCC
AGGCTCCAGGGAAGGGGCTtGAGTGGGTCTCAGATATTAATAATGAAGGTGGTACTA
CATACTATGCAGACTCCGTGAAGGGCCGATTCACCATCTCCAGAGACAACTCTAAG
AACAGCCTGTATCTGCAAATGAACAGCCTGCGCGCCGAGGACACGGCCGTGTACTA
CTGCGCGAGAGATGCCGGATATAGCAACCATGTACCCATCTTTGATTCTTGGGGCCA
GGGGACCCTGGTCACTGTCTCCTCACGTACGGTCACTGTCTCTTCACAGGATCCCGC
CGAGCCCAAATCTCCTGACAAAACTCACACATGCCCACCGTGCCCAGCACCTGAACT
CCTGGGGGGACCGTCAGTCTTCCTCTTCCCCCCAAAACCCAAGGACACCCTCATGAT
CTCCCGGACCCCTGAGGTCACATGCGTGGTGGTGGACGTGAGCCACGAAGACCCTG
AGGTCAAGTTCAACTGGTACGTGGACGGCGTGGAGGTGCATAATGCCAAGACAAAG
CCGCGGGAGGAGCAGTACAACAGCACGTACCGTGTGGTCAGCGTCCTCACCGTCCT
GCACCAGGACTGGCTGAATGGCAAGGAGTACAAGTGCAAGGTCTCCAACAAAGCCC
TCCCAGCCCCCATCGAGAAAACCATCTCCAAAGCCAAAGGGCAGCCCCGAGAACCA
CAGGTGTACACCCTGCCCCCATCCCGGGATGAGCTGACCAAGAACCAGGTCAGCCT
GACCTGCCTGGTCAAAGGCTTCTATCCCAGCGACATCGCCGTGGAGTGGGAGAGCA
ATGGGCAACCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAA
CGTCTTCTCATGCTCCGTGATGCATGAGGCTCTGCACAACCACTACACGCAGAAGAG
CCTCTCCCTGTCTCCGGGTAAAAAAGATCCCAAATTTTGGGTGCTGGTGGTGGTTGG
TGGAGTCCTGGCTTGCTATAGCTTGCTAGTAACAGTGGCCTTTATTATTTTCTGGGTG
AGGAGTAAGAGGAGCAGGCTCCTGCACAGTGACTACATGAACATGACTCCCCGCCG
CCCCGGGCCCACCCGCAAGCATTACCAGCCCTATGCCCCACCACGCGACTTCGCAGC
CTATCGCTCACGCGTGAAGTTCAGCAGGAGCGCAGACGCCCCCGCGTACCAGCAGG
GCCAGAACCAGCTCTATAACGAGCTCAATCTAGGACGAAGAGAGGAGTACGATGTT
TTGGACAAAAGACGTGGCCGGGACCCTGAGATGGGGGGAAAGCCGAGAAGGAAGA
ACCCTCAGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGGCCTTT
ACCAGGGTCTCAGTACAGCCACCAAGGACACCTACGACGCCCTTCACATGCAGGCC
CTGCCCCCTCGCGGACCGCAGTGTACTAATTATGCTCTCTTGAAATTGGCTGGAGAT
GTTGAGAGCAATCCCGGGCCCATGCGCATTAGCAAGCCCCACCTGCGGAGCATCAG
CATCCAGTGCTACCTGTGCCTGCTGCTGAACAGCCACTTCCTGACCGAGGCCGGCAT
CCACGTGTTCATCCTGGGCTGCTTCAGCGCCGGACTGCCCAAGACCGAGGCCAACTG
GGTGAACGTGATCAGCGACCTGAAGAAGATCGAGGACCTGATCCAGAGCATGCACA
TCGACGCCACCCTGTACACCGAGAGCGACGTGCACCCCAGCTGCAAGGTGACCGCC
ATGAAGTGCTTTCTGCTGGAACTGCAGGTGATCAGCCTGGAAAGCGGCGACGCCAG
CATCCACGACACCGTGGAGAACCTGATCATCCTGGCCAACAACAGCCTGAGCAGCA
ACGGCAACGTGACCGAGAGCGGCTGCAAAGAGTGCGAGGAACTGGAAGAGAAGAA
CATCAAAGAGTTTCTGCAGAGCTTCGTGCACATCGTGCAGATGTTCATCAACACCAG
CTGA.

In some cases, a particular antibody having a CD8alpha signal peptide (CD8SP) is utilized. One example of a CD8SP CD70 42D12 VLVH sequence is as follows:

DNA
(SEQ ID NO: 10)
ATGGCCCTGCCTGTGACAGCTCTGCTCCTCCCTCTGGCCCTGCTGCTCC
ATGCCGCCAGACCCCAGGCAGTGGTGACCCAGGAGCCTTCCCTGACAGT
GTCTCCAGGAGGGACGGTCACGCTCACCTGCGGCCTCAAATCTGGGTCT
GTCACTTCCGATAACTTCCCCACTTGGTACCAGCAGACACCAGGCCAGG
CTCCCCGATTGCTTATCTACAACACAAACACCCGTCACTCTGGCGTCCC
CGACCGCTTCTCCGGATCCATCCTGGGCAACAAAGCCGCCCTCACCATC
ACGGGGGCCCAGGCCGACGACGAGGCCGAATATTTCTGTGCTCTGTTCA
TAAGTAATCCTAGTGTTGAGTTCGGCGGAGGGACCCAACTGACCGTCCT
AGGTGGCAGCACCAGCGGCTCCGGCAAGCCTGGCTCTGGCGAGGGCAGC
ACAAAGGGAGAGGTGCAGCTCGTGGAGTCTGGGGGAGGCTTGGTGCAGC
CTGGGGGGTCTCTGAGACTCTCCTGTGCAGCCTCTGGATTCACCTTCAG
TGTCTACTACATGAACTGGGTCCGCCAGGCTCCAGGGAAGGGGCTCGAG
TGGGTCTCAGATATTAATAATGAAGGTGGTACTACATACTATGCAGACT
CCGTGAAGGGCCGATTCACCATCTCCAGAGACAACTCTAAGAACAGCCT
GTATCTGCAAATGAACAGCCTGCGCGCCGAGGACACGGCCGTGTACTAC
TGCGCGAGAGATGCCGGATATAGCAACCATGTACCCATCTTTGATTCTT
GGGGCCAGGGGACCCTGGTCACTGTCTCCTCA
Protein
(SEQ ID NO: 11)
ARVATMGMALPVTALLLPLALLLHAARPQAVVTQEPSLTVSPGGTVTLT
CGLKSGSVTSDNFPTWYQQTPGQAPRLLIYNTNTRHSGVPDRFSGSILG
NKAALTITGAQADDEAEYFCALFISNPSVEFGGGTQLTVLGGSTSGSGK
PGSGEGSTKGEVQLVESGGGLVQPGGSLRLSCAASGFTFSVYYMNWVRQ
APGKGLEWVSDINNEGGTTYYADSVKGRFTISRDNSKNSLYLQMNSLRA
EDTAVYYCARDAGYSNHVPIFDSWGQGTLVTVSS

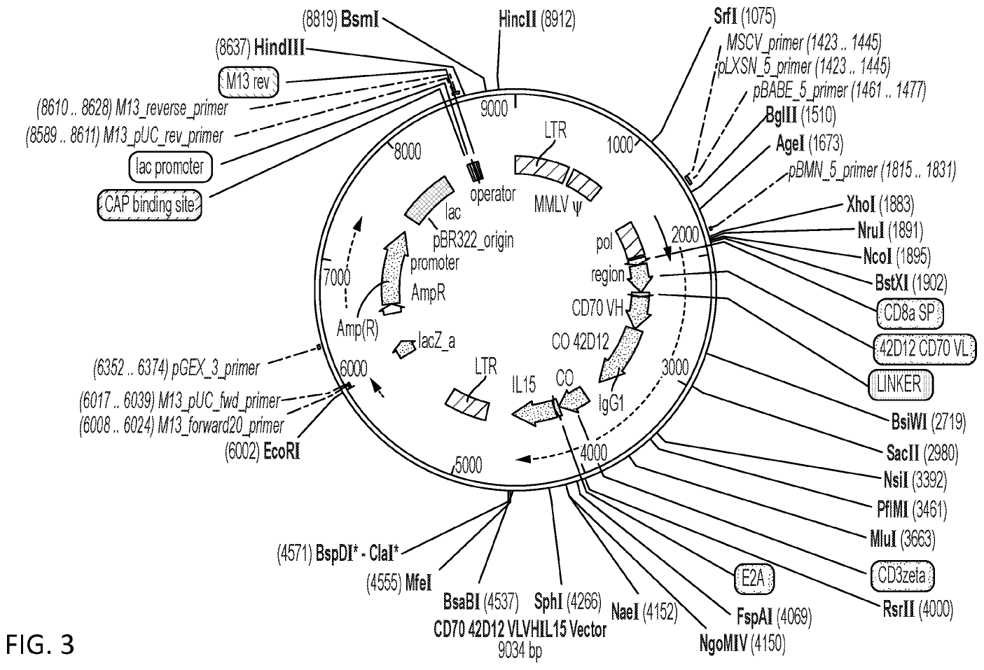

A plasmid vector map for an example CAR of CAR.CD70 42D12 VLVH.IgG1.CD28.CD3z-2A-IL15 is provided in FIG. 3.

The full DNA sequence for CAR.CD70 42D12 VLVH.IgG1.CD28.CD3z-2A-IL15 is as follows:

(SEQ ID NO: 12)
ATGGCCCTGCCTGTGACAGCTCTGCTCCTCCCTCTGGCCCTGCTGCTCCATGC
CGCCAGACCCCAGGCAGTtGTGACCCAGGAGCCTTCCCTGACAGTGTCTCCAGGAGG
GACGGTCACGCTCACCTGCGGCCTCAAATCTGGGTCTGTCACTTCCGATAACTTCCC
CACTTGGTACCAGCAGACACCAGGCCAGGCTCCCCGATTGCTTATCTACAACACAAA
CACCCGTCACTCTGGCGTCCCCGACCGCTTCTCCGGATCCATCCTGGGCAACAAAGC
CGCCCTCACCATCACGGGGGCCCAGGCCGACGACGAGGCCGAATATTTCTGTGCTCT
GTTCATAAGTAATCCTAGTGTTGAGTTCGGCGGAGGGACCCAACTGACCGTCCTAGG
TGGCAGCACCAGCGGCTCCGGCAAGCCTGGCTCTGGCGAGGGCAGCACAAAGGGAG
AGGTGCAGCTCGTGGAGTCTGGGGGAGGCTTGGTGCAGCCTGGGGGGTCTCTGAGA
CTCTCCTGTGCAGCCTCTGGATTCACCTTCAGTGTCTACTACATGAACTGGGTCCGCC
AGGCTCCAGGGAAGGGGCTtGAGTGGGTCTCAGATATTAATAATGAAGGTGGTACTA
CATACTATGCAGACTCCGTGAAGGGCCGATTCACCATCTCCAGAGACAACTCTAAG
AACAGCCTGTATCTGCAAATGAACAGCCTGCGCGCCGAGGACACGGCCGTGTACTA
CTGCGCGAGAGATGCCGGATATAGCAACCATGTACCCATCTTTGATTCTTGGGGCCA
GGGGACCCTGGTCACTGTCTCCTCACGTACGGTCACTGTCTCTTCACAGGATCCCGC
CGAGCCCAAATCTCCTGACAAAACTCACACATGCCCACCGTGCCCAGCACCTGAACT
CCTGGGGGGACCGTCAGTCTTCCTCTTCCCCCCAAAACCCAAGGACACCCTCATGAT
CTCCCGGACCCCTGAGGTCACATGCGTGGTGGTGGACGTGAGCCACGAAGACCCTG
AGGTCAAGTTCAACTGGTACGTGGACGGCGTGGAGGTGCATAATGCCAAGACAAAG
CCGCGGGAGGAGCAGTACAACAGCACGTACCGTGTGGTCAGCGTCCTCACCGTCCT
GCACCAGGACTGGCTGAATGGCAAGGAGTACAAGTGCAAGGTCTCCAACAAAGCCC
TCCCAGCCCCCATCGAGAAAACCATCTCCAAAGCCAAAGGGCAGCCCCGAGAACCA
CAGGTGTACACCCTGCCCCCATCCCGGGATGAGCTGACCAAGAACCAGGTCAGCCT
GACCTGCCTGGTCAAAGGCTTCTATCCCAGCGACATCGCCGTGGAGTGGGAGAGCA
ATGGGCAACCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAA
CGTCTTCTCATGCTCCGTGATGCATGAGGCTCTGCACAACCACTACACGCAGAAGAG
CCTCTCCCTGTCTCCGGGTAAAAAAGATCCCAAATTTTGGGTGCTGGTGGTGGTTGG
TGGAGTCCTGGCTTGCTATAGCTTGCTAGTAACAGTGGCCTTTATTATTTTCTGGGTG
AGGAGTAAGAGGAGCAGGCTCCTGCACAGTGACTACATGAACATGACTCCCCGCCG
CCCCGGGCCCACCCGCAAGCATTACCAGCCCTATGCCCCACCACGCGACTTCGCAGC
CTATCGCTCACGCGTGAAGTTCAGCAGGAGCGCAGACGCCCCCGCGTACCAGCAGG
GCCAGAACCAGCTCTATAACGAGCTCAATCTAGGACGAAGAGAGGAGTACGATGTT
TTGGACAAAAGACGTGGCCGGGACCCTGAGATGGGGGGAAAGCCGAGAAGGAAGA
ACCCTCAGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGGCCTTT
ACCAGGGTCTCAGTACAGCCACCAAGGACACCTACGACGCCCTTCACATGCAGGCC
CTGCCCCCTCGCGGACCGCAGTGTACTAATTATGCTCTCTTGAAATTGGCTGGAGAT
GTTGAGAGCAATCCCGGGCCCATGCGCATTAGCAAGCCCCACCTGCGGAGCATCAG
CATCCAGTGCTACCTGTGCCTGCTGCTGAACAGCCACTTCCTGACCGAGGCCGGCAT
CCACGTGTTCATCCTGGGCTGCTTCAGCGCCGGACTGCCCAAGACCGAGGCCAACTG
GGTGAACGTGATCAGCGACCTGAAGAAGATCGAGGACCTGATCCAGAGCATGCACA
TCGACGCCACCCTGTACACCGAGAGCGACGTGCACCCCAGCTGCAAGGTGACCGCC
ATGAAGTGCTTTCTGCTGGAACTGCAGGTGATCAGCCTGGAAAGCGGCGACGCCAG
CATCCACGACACCGTGGAGAACCTGATCATCCTGGCCAACAACAGCCTGAGCAGCA
ACGGCAACGTGACCGAGAGCGGCTGCAAAGAGTGCGAGGAACTGGAAGAGAAGAA
CATCAAAGAGTTTCTGCAGAGCTTCGTGCACATCGTGCAGATGTTCATCAACACCAG
CTGA

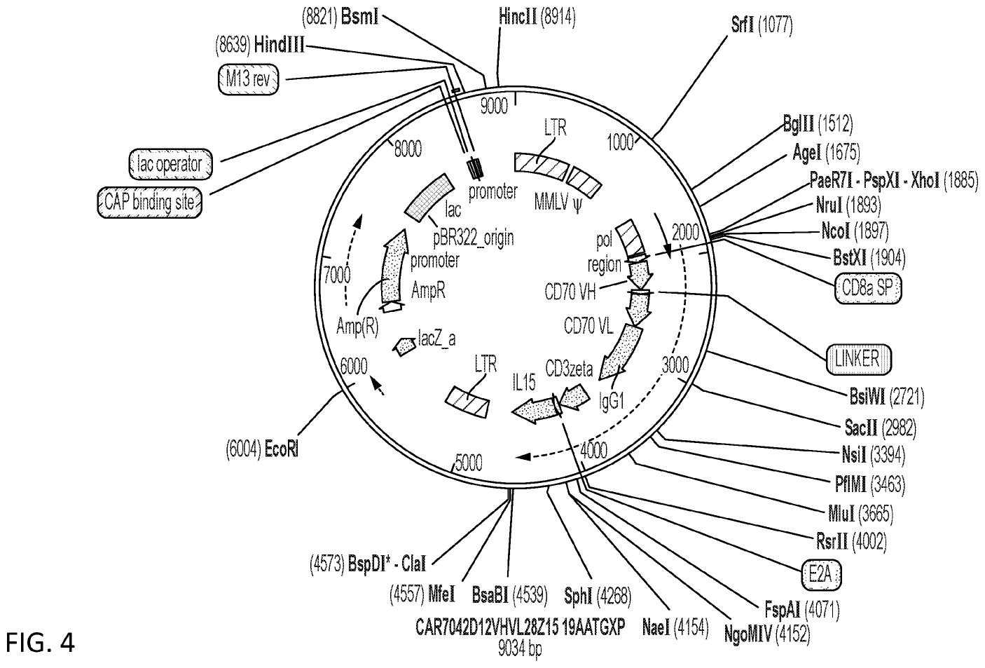

A CAR.CD70 42D12 VHVL.IgG1.CD28.CD3z-2A-IL15 vector may be utilized in the methods and compositions of the disclosure. A plasmid vector map for CAR.CD70 42D12 VHVL.IgG1.CD28.CD3z-2A-IL15 is illustrated in FIG. 4. A full DNA sequence of CAR.CD70 42D12 VHVL.IgG1.CD28.CD3z-2A-IL15 is as follows:

(SEQ ID NO: 13)
ATGGCCCTGCCTGTGACAGCTCTGCTCCTCCCTCTGGCCCTGCTGCTCCATGC
CGCCAGACCCGAGGTGCAGCTCGTGGAGTCTGGGGGAGGCTTGGTGCAGCCTGGGG
GGTCTCTGAGACTCTCCTGTGCAGCCTCTGGATTCACCTTCAGTGTCTACTACATGAA
CTGGGTCCGCCAGGCTCCAGGGAAGGGGCTTGAGTGGGTCTCAGATATTAATAATG
AAGGTGGTACTACATACTATGCAGACTCCGTGAAGGGCCGATTCACCATCTCCAGAG
ACAACTCTAAGAACAGCCTGTATCTGCAAATGAACAGCCTGCGCGCCGAGGACACG
GCCGTGTACTACTGCGCGAGAGATGCCGGATATAGCAACCATGTACCCATCTTTGAT
TCTTGGGGCCAGGGGACCCTGGTCACTGTCTCCTCAGGCAGCACCAGCGGCTCCGGC
AAGCCTGGCTCTGGCGAGGGCAGCACAAAGGGACAGGCAGTGGTGACCCAGGAGC
CTTCCCTGACAGTGTCTCCAGGAGGGACGGTCACGCTCACCTGCGGCCTCAAATCTG
GGTCTGTCACTTCCGATAACTTCCCCACTTGGTACCAGCAGACACCAGGCCAGGCTC
CCCGATTGCTTATCTACAACACAAACACCCGTCACTCTGGCGTCCCCGACCGCTTCT
CCGGATCCATCCTGGGCAACAAAGCCGCCCTCACCATCACGGGGGCCCAGGCCGAC
GACGAGGCCGAATATTTCTGTGCTCTGTTCATAAGTAATCCTAGTGTTGAGTTCGGC
GGAGGGACCCAACTGACCGTCCTAGGTCGTACGGTCACTGTCTCTTCACAGGATCCC
GCCGAGCCCAAATCTCCTGACAAAACTCACACATGCCCACCGTGCCCAGCACCTGA
ACTCCTGGGGGGACCGTCAGTCTTCCTCTTCCCCCCAAAACCCAAGGACACCCTCAT
GATCTCCCGGACCCCTGAGGTCACATGCGTGGTGGTGGACGTGAGCCACGAAGACC
CTGAGGTCAAGTTCAACTGGTACGTGGACGGCGTGGAGGTGCATAATGCCAAGACA
AAGCCGCGGGAGGAGCAGTACAACAGCACGTACCGTGTGGTCAGCGTCCTCACCGT
CCTGCACCAGGACTGGCTGAATGGCAAGGAGTACAAGTGCAAGGTCTCCAACAAAG
CCCTCCCAGCCCCCATCGAGAAAACCATCTCCAAAGCCAAAGGGCAGCCCCGAGAA
CCACAGGTGTACACCCTGCCCCCATCCCGGGATGAGCTGACCAAGAACCAGGTCAG
CCTGACCTGCCTGGTCAAAGGCTTCTATCCCAGCGACATCGCCGTGGAGTGGGAGA
GCAATGGGCAACCGGAGAACAACTACAAGACCACGCCTCCCGTGCTGGACTCCGAC
GGCTCCTTCTTCCTCTACAGCAAGCTCACCGTGGACAAGAGCAGGTGGCAGCAGGG
GAACGTCTTCTCATGCTCCGTGATGCATGAGGCTCTGCACAACCACTACACGCAGAA
GAGCCTCTCCCTGTCTCCGGGTAAAAAAGATCCCAAATTTTGGGTGCTGGTGGTGGT
TGGTGGAGTCCTGGCTTGCTATAGCTTGCTAGTAACAGTGGCCTTTATTATTTTCTGG
GTGAGGAGTAAGAGGAGCAGGCTCCTGCACAGTGACTACATGAACATGACTCCCCG
CCGCCCCGGGCCCACCCGCAAGCATTACCAGCCCTATGCCCCACCACGCGACTTCGC
AGCCTATCGCTCACGCGTGAAGTTCAGCAGGAGCGCAGACGCCCCCGCGTACCAGC
AGGGCCAGAACCAGCTCTATAACGAGCTCAATCTAGGACGAAGAGAGGAGTACGAT
GTTTTGGACAAAAGACGTGGCCGGGACCCTGAGATGGGGGGAAAGCCGAGAAGGA
AGAACCCTCAGGAAGGCCTGTACAATGAACTGCAGAAAGATAAGATGGCGGAGGCC
TACAGTGAGATTGGGATGAAAGGCGAGCGCCGGAGGGGCAAGGGGCACGATGGCC
TTTACCAGGGTCTCAGTACAGCCACCAAGGACACCTACGACGCCCTTCACATGCAGG
CCCTGCCCCCTCGCGGACCGCAGTGTACTAATTATGCTCTCTTGAAATTGGCTGGAG
ATGTTGAGAGCAATCCCGGGCCCATGCGCATTAGCAAGCCCCACCTGCGGAGCATC
AGCATCCAGTGCTACCTGTGCCTGCTGCTGAACAGCCACTTCCTGACCGAGGCCGGC
ATCCACGTGTTCATCCTGGGCTGCTTCAGCGCCGGACTGCCCAAGACCGAGGCCAAC
TGGGTGAACGTGATCAGCGACCTGAAGAAGATCGAGGACCTGATCCAGAGCATGCA
CATCGACGCCACCCTGTACACCGAGAGCGACGTGCACCCCAGCTGCAAGGTGACCG
CCATGAAGTGCTTTCTGCTGGAACTGCAGGTGATCAGCCTGGAAAGCGGCGACGCC
AGCATCCACGACACCGTGGAGAACCTGATCATCCTGGCCAACAACAGCCTGAGCAG
CAACGGCAACGTGACCGAGAGCGGCTGCAAAGAGTGCGAGGAACTGGAAGAGAAG
AACATCAAAGAGTTTCTGCAGAGCTTCGTGCACATCGTGCAGATGTTCATCAACACC
AGCTGA

C. T Cell Receptor (TCR)

In some embodiments, the genetically engineered antigen receptors include recombinant TCRs and/or TCRs cloned from naturally occurring T cells. A “T cell receptor” or “TCR” refers to a molecule that contains a variable a and β chains (also known as TCRα and TCRβ, respectively) or a variable γ and δ chains (also known as TCRγ and TCRδ, respectively) and that is capable of specifically binding to an antigen peptide bound to a MHC receptor. In some embodiments, the TCR is in the αβ form.

Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., a-chain, β-chain) can contain two immunoglobulin domains, a variable domain (e.g., Vₐ or Vp; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., a-chain constant domain or Cₐ, typically amino acids 117 to 259 based on Kabat, β-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3 chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al., 2009 and Cohen et al., 2005). In some embodiments, phage display is used to isolate TCRs against a target antigen (see, e.g., Varela-Rohena et al., 2008 and Li, 2005). In some embodiments, the TCR or antigen-binding portion thereof can be synthetically generated from knowledge of the sequence of the TCR.

### II. CYTOKINES

One or more cytokines may be utilized with one or more CD70-targeting genetically engineered receptors, such as CD70-specific CARs. In some cases, one or more cytokines are present on the same vector molecule as the engineered receptor, although in other cases they are on separate molecules. In particular embodiments, one or more cytokines are co-expressed from the same vector as the engineered receptor. One or more cytokines may be produced as a separate polypeptide from the CD70-specific receptor. As one example, Interleukin-15 (IL-15), is utilized. IL-15 may be employed because, for example, it is tissue restricted and only under pathologic conditions is it observed at any level in the serum, or systemically. IL-15 possesses several attributes that are desirable for adoptive therapy. IL-15 is a homeostatic cytokine that induces development and cell proliferation of natural killer cells, promotes the eradication of established tumors via alleviating functional suppression of tumor-resident cells, and inhibits activation-induced cell death. In addition to IL-15, other cytokines are envisioned. These include, but are not limited to, cytokines, chemokines, and other molecules that contribute to the activation and proliferation of cells used for human application. As one example, the cytokine is IL-15, IL-12, IL-2, IL-18, IL-21, IL-7, or combination thereof. NK cells expressing IL-15 may be utilized and are capable of continued supportive cytokine signaling, which is useful for their survival post-infusion.

In specific embodiments, NK cells expresses one or more exogenously provided cytokines. The cytokine may be exogenously provided to the NK cells because it is expressed from an expression vector within the cell. In an alternative case, an endogenous cytokine in the cell is upregulated upon manipulation of regulation of expression of the endogenous cytokine, such as genetic recombination at the promoter site(s) of the cytokine. In cases wherein the cytokine is provided on an expression construct to the cell, the cytokine may be encoded from the same vector as a suicide. The cytokine may be expressed as a separate polypeptide molecule as a suicide gene and as a separate polypeptide from an engineered receptor of the cell. In some embodiments, the present disclosure concerns co-utilization of CAR and/or TCR vectors with IL-15, particularly in NK cells.

### III. SUICIDE GENES

In particular embodiments, a suicide gene is utilized in conjunction with cell therapy of any kind to control its use and allow for termination of the cell therapy at a desired event and/or time. The suicide gene is employed in transduced cells for the purpose of eliciting death for the transduced cells when needed. The CD70-targeting cells of the present disclosure that have been modified to harbor a vector encompassed by the disclosure may comprise one or more suicide genes. In some embodiments, the term “suicide gene” as used herein is defined as a gene which, upon administration of a prodrug or other agent, effects transition of a gene product to a compound which kills its host cell. In other embodiments, a suicide gene encodes a gene product that is, when desired, targeted by an agent (such as an antibody) that targets the suicide gene product.

Examples of suicide gene/prodrug combinations which may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside. The E. coli purine nucleoside phosphorylase, a so-called suicide gene that converts the prodrug 6-methylpurine deoxyriboside to toxic purine 6-methylpurine, may be used. Other examples of suicide genes used with prodrug therapy are the E. coli cytosine deaminase gene and the HSV thymidine kinase gene.

Exemplary suicide genes also include CD20, CD52, EGFRv3, or inducible caspase 9. In one embodiment, a truncated version of EGFR variant III (EGFRv3) may be used as a suicide antigen that can be ablated by Cetuximab. Further suicide genes known in the art that may be used in the present disclosure include Purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), and Thymidine phosphorylase (TP).

In particular embodiments, vectors that encode the CD70-targeting CAR, or any vector in a NK cell encompassed herein, include one or more suicide genes. The suicide gene may or may not be on the same vector as a CD70-targeting CAR. In cases wherein the suicide gene is present on the same vector as the CD70-targeting CAR, the suicide gene and the CAR may be separated by an IRES or 2A element, for example.

In specific embodiments, the suicide gene is a tumor necrosis factor (TNF)-alpha mutant that is uncleavable by standard enzymes that cleave TNF in nature, such as TNF-alpha-converting enzyme (also referred to as TACE). As such, the TNF-alpha mutant is membrane-bound and nonsecretable, in particular embodiments. The TNF-alpha mutant used in the disclosure is targetable by one or more agents that bind the mutant, including at least an antibody, such that following binding of the agent(s) to the TNF-alpha mutant on the surface of the cell, the cell dies. Embodiments of the disclosure allow the TNF-alpha mutant to be utilized as a marker for cells that express it.

Cells expressing the uncleavable TNF-alpha mutants can be targeted for selective deletion including, for example, using FDA-approved TNF-α antibodies currently in the clinic, such as etanercept, infliximab or adalilumab. The mutated TNF-alpha polypeptide may be co-expressed with one or more therapeutic transgenes in the cell, such as a gene encoding a TCR or CAR, including CD70-targeting TCRs and/or CARs. In addition, the TNF-alpha mutant expressing cells have superior activity against the tumor target, mediated by the biological activity of the membrane-bound TNF-alpha protein.

With respect to wild-type, TNF-alpha has a 26 kD transmembrane form and a 17 kD secretory component. Some mutants described in Perez et al. (1990) may be utilized in the disclosure. In specific embodiments, examples of TNF-alpha mutants of the disclosure include at least the following with respect to the 17 kD TNF: (1) deletion of Val1 and deletion of Prol12; (2) deletion of Val13; (3) deletion of Val1 and deletion of Val13; (4) deletion of Val1 through and including Prol12 and deletion of Val13 (delete 13aa); (5) deletion of Ala-3 through to and including Val 13 (delete 14 aa). In specific embodiments, a TNF-alpha mutant comprises deletion of the respective amino acid at position −3, −2, −1, 1, 2, 3, 4, 5, 6, 7, 8, 9, 10, 11, 12, 13, or a combination thereof. Specific combinations include deletions at positions −3 through and including 13; −3 through and including 12; −3 through and including 11; −3 through and including 10; −3 through and including 9; −3 through and including 8; −3 through and including 7; −3 through and including 6; −3 through and including 5; −3 through and including 4; −3 through and including 3; −3 through and including 2; −3 through and including 1; −3 through and including −1; −3 through and including −2; −2 through and including 13; −2 through and including 12; −2 through and including 11; −2 through and including 10; −2 through and including 9; −2 through and including 8; −2 through and including 7; −2 through and including 6; −2 through and including 5; −2 through and including 4; −2 through and including 3; −2 through and including 2; −2 through and including 1; −2 through and including −1; −1 through and including 13; −1 through and including 12; −1 through and including 11; −1 through and including 10; −1 through and including 9; −1 through and including 8; −1 through and including 7; −1 through and including 6; −1 through and including 5; −1 through and including 4; −1 through and including 3; −1 through and including 2; −1 through and including 1; 1 through and including 13; 1 through and including 12; 1 through and including 11; 1 through and including 10; 1 through and including 9; 1 through and including 8; 1 through and including 7; 1 through and including 6; 1 through and including 5; 1 through and including 4; 1 through and including 3; 1 through and including 2; and so forth.

The TNF-alpha mutants may be generated by any suitable method, but in specific embodiments they are generated by site-directed mutagenesis. In some cases, the TNF-alpha mutants may have mutations other than those that render the protein uncleavable. In specific cases, the TNF-alpha mutants may have 1, 2, 3, or more mutations other than the deletions at Val1, Pro12, and/or Val13 or the region there between. The mutations other than those that render the mutants nonsecretable may be one or more of an amino acid substitution, deletion, addition, inversion, and so forth. In cases wherein the additional mutation is an amino acid substitution, the substitution may or may not be to a conservative amino acid, for example. In some cases, 1, 2, 3, 4, 5, or more additional amino acids may be present on the N-terminal and/or C-terminal ends of the protein. In some cases, a TNF-alpha mutant has (1) one or more mutations that render the mutant nonsecretable; (2) one or more mutations that prevents outside-in signaling for the mutant; and/or (3) one or more mutations that interfere with binding of the mutant to TNF Receptor 1 and/or TNF Receptor 2.

In particular embodiments, upon delivering an effective amount of one or more agents to bind to the TNF-alpha mutant-expressing CD70 CAR-targeting cells, the majority of TNF-alpha mutant-expressing cells are eliminated. In specific embodiments, greater than 50%, 55%, 60%, 65%, 70%, 75%, 80%, 85%, 90%, 95%, 96%, 97%, 98%, or 99% of cells expressing the TNF-alpha mutants are eliminated in an individual. Following recognition of a need to eliminate the cells, the delivery of the agent(s) to the individual may continue until one or more symptoms are no longer present or until a sufficient number of cells have been eliminated. The cell numbers in the individual may be monitored using the TNF-alpha mutants as markers.

Embodiments of methods of the disclosure may comprise a first step of providing an effective amount of the CD70-targeting immune cell therapy to an individual in need thereof, wherein the cells comprise one or more nonsecretable TNF-alpha mutants; and, a second step of eliminating the cells using the TNF-alpha mutant(s) as suicide genes (directly or indirectly through cell death by any mechanism). The second step may be instigated upon onset of at least one adverse event for the individual, and that adverse event may be recognized by any means, including upon routine monitoring that may or may not be continuous from the beginning of the cell therapy. The adverse event(s) may be detected upon examination and/or testing. In cases wherein the individual has cytokine release syndrome (which may also be referred to as cytokine storm), the individual may have elevated inflammatory cytokine(s) (merely as examples: interferon-gamma, granulocyte macrophage colony-stimulating factor, IL-10, IL-6 and TNF-alpha); fever; fatigue; hypotension; hypoxia, tachycardia; nausea; capillary leak; cardiac/renal/hepatic dysfunction; or a combination thereof, for example. In cases wherein the individual has neurotoxicity, the individual may have confusion, delirium, aplasia, and/or seizures. In some cases, the individual is tested for a marker associated with onset and/or severity of cytokine release syndrome, such as C-reactive protein, IL-6, TNF-alpha, and/or ferritin

In additional embodiments, administration of one or more agents that bind the nonsecretable TNF-α during cytokine release syndrome or neurotoxicity, for example, have the added benefit of neutralizing the high levels of soluble TNF-alpha that contribute to the toxicity of the therapy. Soluble TNF-alpha is released at high levels during cytokine release syndrome and is a mediator of toxicity with CAR T-cell therapies. In such cases, the administration of TNF-alpha antibodies encompassed herein have a dual beneficial effect—i.e. selective deletion of the TNF-alpha mutant-expressing cells as well as neutralizing soluble TNF-alpha causing toxicity. Thus, embodiments of the disclosure encompass methods of eliminating or reducing the severity of cytokine release syndrome in an individual receiving, or who has received, adoptive cell therapy in which the cells express a nonsecretable TNF-alpha mutant, comprising the step of providing an effective amount of an agent that binds the nonsecretable TNF-alpha mutant, said agent causing in the individual (a) elimination of at least some of the cells of the cell therapy; and (b) reduction in levels of soluble TNF-alpha.

Embodiments of the disclosure include methods of reducing the effects of cytokine release syndrome in an individual that has received or who is receiving cell therapy with cells that express a nonsecretable TNF-alpha mutant, comprising the step of providing an effective amount of one or more agents that bind the mutant to cause in the individual (a) elimination of at least some of the cells of the cell therapy; and (b) reduction in the level of soluble TNF1-alpha.

When the need arises for the TNF-alpha suicide gene to be utilized, the individual is provided an effective amount of one or more inhibitors that are able to inhibit, such as by binding directly, the TNF-alpha mutant on the surface of the cells. The inhibitor(s) may be provided to the individual systemically and/or locally in some embodiments. The inhibitor may be a polypeptide (such as an antibody), a nucleic acid, a small molecule (for example, a xanthine derivative), a peptide, or a combination thereof. In specific embodiments, the antibodies are FDA-approved. When the inhibitor is an antibody, the inhibitor may be a monoclonal antibody in at least some cases. When mixtures of antibodies are employed, one or more antibodies in the mixture may be a monoclonal antibody. Examples of small molecule TNF-alpha inhibitors include small molecules such as are described in U.S. Pat. No. 5,118,500, which is incorporated by reference herein in its entirety. Examples of polypeptide TNF-alpha inhibitors include polypeptides, such as those described in U.S. Pat. No. 6,143,866, which is incorporated by reference herein in its entirety.

In particular embodiments, at least one antibody is utilized to target the TNF-alpha mutant to trigger its activity as a suicide gene. Examples of antibodies includes at least Adalimumab, Adalimumab-atto, Certolizumab pegol, Etanercept, Etanercept-szzs, Golimumab, Infliximab, Infliximab-dyyb, or a mixture thereof, for example.

Embodiments of the disclosure include methods of reducing the risk of toxicity of a cell therapy for an individual by modifying cells of a cell therapy to express a nonsecretable TNF-alpha mutant. The cell therapy is for cancer, in specific embodiments, and it may comprise an engineered receptor that targets an antigen, including a cancer antigen.

In particular embodiments, in addition to the inventive NK cell therapy of the disclosure, the individual may have been provided, may be provided, and/or may will be provided an additional therapy for the medical condition. In cases wherein the medical condition is cancer, the individual may be provided one or more of surgery, radiation, immunotherapy (other than the cell therapy of the present disclosure), hormone therapy, gene therapy, chemotherapy, and so forth.

### IV. VECTORS

The CD70-targeting CARs may be delivered to the recipient immune cells by any suitable vector, including by a viral vector or by a non-viral vector. Examples of viral vectors include at least retroviral, lentiviral, adenoviral, or adeno-associated viral vectors. Examples of non-viral vectors include at least plasmids, transposons, lipids, nanoparticles, and so forth.

In cases wherein the immune cell is transduced with a vector encoding the CD70-targeting receptor and also requires transduction of another gene or genes into the cell, such as a suicide gene and/or cytokine and/or an optional therapeutic gene product, the CD70-targeting receptor, suicide gene, cytokine, and optional therapeutic gene may or may not be comprised on or with the same vector. In some cases, the CD70-targeting CAR, suicide gene, cytokine, and optional therapeutic gene are expressed from the same vector molecule, such as the same viral vector molecule. In such cases, the expression of the CD70-targeting CAR, suicide gene, cytokine, and optional therapeutic gene may or may not be regulated by the same regulatory element(s). When the CD70-targeting CAR, suicide gene, cytokine, and optional therapeutic gene are on the same vector, they may or may not be expressed as separate polypeptides. In cases wherein they are expressed as separate polypeptides, they may be separated on the vector by a 2A element or IRES element (or both kinds may be used on the same vector once or more than once), for example.

### A. General Embodiments

One of skill in the art would be well-equipped to construct a vector through standard recombinant techniques (see, for example, Sambrook et al., 2001 and Ausubel et al., 1996, both incorporated herein by reference) for the expression of the antigen receptors of the present disclosure.

1. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells may be comprised of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters, for example. In cases wherein the vector is utilized for the generation of cancer therapy, a promoter may be effective under conditions of hypoxia.

2. Promoter/Enhancers

The expression constructs provided herein comprise a promoter to drive expression of the antigen receptor and other cistron gene products. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, for example, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter, GADPH promoter, metallothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at GenBank®, accession no. X05244, nucleotide 283-341) or a mouse mammary tumor promoter (available from the ATCC, Cat. No. ATCC 45007). In certain embodiments, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

3. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

As detailed elsewhere herein, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the equine rhinitis A virus (E2A) or the F2A (Foot-and-mouth disease virus 2A) or a “2A-like” sequence (e.g., Thosea asigna virus 2A; T2A) or porcine teschovirus-1 (P2A). In specific embodiments, in a single vector the multiple 2A sequences are non-identical, although in alternative embodiments the same vector utilizes two or more of the same 2A sequences. Examples of 2A sequences are provided in US 2011/0065779 which is incorporated by reference herein in its entirety.

4. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively a replication origin of other extra-chromosomally replicating virus as described above or an autonomously replicating sequence (ARS) can be employed.

5. Selection and Screenable Markers

In some embodiments, NK cells comprising a CD70-targeting receptor construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

### B. Multicistronic Vectors

In particular embodiments, the CD70-targeting receptor, optional suicide gene, optional cytokine, and/or optional therapeutic gene are expressed from a multicistronic vector (The term “cistron” as used herein refers to a nucleic acid sequence from which a gene product may be produced). In specific embodiments, the multicistronic vector encodes the CD70-targeting receptor, the suicide gene, and at least one cytokine, and/or engineered receptor, such as a T-cell receptor and/or an additional non-CD70-targeting CAR. In some cases, the multicistronic vector encodes at least one CD70-targeting CAR, at least one TNF-alpha mutant, and at least one cytokine. The cytokine may be of a particular type of cytokine, such as human or mouse or any species. In specific cases, the cytokine is IL15, IL12, IL2, IL18, and/or IL21.

In certain embodiments, the present disclosure provides a flexible, modular system (the term “modular” as used herein refers to a cistron or component of a cistron that allows for interchangeability thereof, such as by removal and replacement of an entire cistron or of a component of a cistron, respectively, for example by using standard recombination techniques) utilizing a polycistronic vector having the ability to express multiple cistrons at substantially identical levels. The system may be used for cell engineering allowing for combinatorial expression (including overexpression) of multiple genes. In specific embodiments, one or more of the genes expressed by the vector includes one, two, or more antigen receptors. The multiple genes may comprise, but are not limited to, CARs, TCRs, cytokines, chemokines, homing receptors, CRISPR/Cas9-mediated gene mutations, decoy receptors, cytokine receptors, chimeric cytokine receptors, and so forth. The vector may further comprise: (1) one or more reporters, for example fluorescent or enzymatic reporters, such as for cellular assays and animal imaging; (2) one or more cytokines or other signaling molecules; and/or (3) a suicide gene.

In specific cases, the vector may comprise at least 4 cistrons separated by cleavage sites of any kind, such as 2A cleavage sites. The vector may or may not be Moloney Murine Leukemia Virus (MoMLV or MMLV)-based including the 3′ and 5′ LTR with the psi packaging sequence in a pUC19 backbone. The vector may comprise 4 or more cistrons with three or more 2A cleavage sites and multiple ORFs for gene swapping. The system allows for combinatorial overexpression of multiple genes (7 or more) that are flanked by restriction site(s) for rapid integration through subcloning, and the system also includes at least three 2A self-cleavage sites, in some embodiments. Thus, the system allows for expression of multiple CARs, TCRs, signaling molecules, cytokines, cytokine receptors, and/or homing receptors. This system may also be applied to other viral and non-viral vectors, including but not limited lentivirus, adenovirus AAV, as well as non-viral plasmids.

The modular nature of the system also enables efficient subcloning of a gene into each of the 4 cistrons in the polycistronic expression vector and the swapping of genes, such as for rapid testing. Restriction sites strategically located in the polycistronic expression vector allow for swapping of genes with efficiency.

Embodiments of the disclosure encompass systems that utilize a polycistronic vector wherein at least part of the vector is modular, for example by allowing removal and replacement of one or more cistrons (or component(s) of one or more cistrons), such as by utilizing one or more restriction enzyme sites whose identity and location are specifically selected to facilitate the modular use of the vector. The vector also has embodiments wherein multiple of the cistrons are translated into a single polypeptide and processed into separate polypeptides, thereby imparting an advantage for the vector to express separate gene products in substantially equimolar concentrations.

The vector of the disclosure is configured for modularity to be able to change one or more cistrons of the vector and/or to change one or more components of one or more particular cistrons. The vector may be designed to utilize unique restriction enzyme sites flanking the ends of one or more cistrons and/or flanking the ends of one or more components of a particular cistron.

Embodiments of the disclosure include polycistronic vectors comprising at least two, at least three, or at least four cistrons each flanked by one or more restriction enzyme sites, wherein at least one cistron encodes for at least one antigen receptor. In some cases, two, three, four, or more of the cistrons are translated into a single polypeptide and cleaved into separate polypeptides, whereas in other cases multiple of the cistrons are translated into a single polypeptide and cleaved into separate polypeptides. Adjacent cistrons on the vector may be separated by a self cleavage site, such as a 2A self cleavage site. In some cases each of the cistrons express separate polypeptides from the vector. On particular cases, adjacent cistrons on the vector are separated by an IRES element.

In certain embodiments, the present disclosure provides a system for cell engineering allowing for combinatorial expression, including overexpression, of multiple cistrons that may include one, two, or more antigen receptors, for example. In particular embodiments, the use of a polycistronic vector as described herein allows for the vector to produce equimolar levels of multiple gene products from the same mRNA. The multiple genes may comprise, but are not limited to, CARs, TCRs, cytokines, chemokines, homing receptors, CRISPR/Cas9-mediated gene mutations, decoy receptors, cytokine receptors, chimeric cytokine receptors, and so forth. The vector may further comprise one or more fluorescent or enzymatic reporters, such as for cellular assays and animal imaging. The vector may also comprise a suicide gene product for termination of cells harboring the vector when they are no longer needed or become deleterious to a host to which they have been provided.

In particular embodiments of the disclosure, at least one of the cistrons on the vector comprises two or more modular components, wherein each of the modular components within a cistron is flanked by one or more restriction enzyme sites. A cistron may comprise three, four, or five modular components, for example. In at least some cases, a cistron encodes an antigen receptor having different parts of the receptor encoded by corresponding modular components. A first modular component of a cistron may encode an antigen binding domain of the receptor. In addition, a second modular component of a cistron may encode a hinge region of the receptor. In addition, a third modular component of a cistron may encode a transmembrane domain of the receptor. In addition, a fourth modular component of a cistron may encode a first costimulatory domain. In addition, a fifth modular component of a cistron may encode a second costimulatory domain. In addition, a sixth modular component of a cistron may encode a signaling domain.

In particular aspects of the disclosure, two different cistrons on the vector each encode non-identical antigen receptors. Both antigen receptors may be encoded by a cistron comprising two or more modular components, including separate cistrons comprising two or more modular components. The antigen receptor may be a chimeric antigen receptor (CAR) and/or T cell receptor (TCR), for example.

In specific embodiments, the vector is a viral vector (retroviral vector, lentiviral vector, adenoviral vector, or adeno-associated viral vector, for example) or a non-viral vector. The vector may comprise a Moloney Murine Leukemia Virus (MMLV) 5′ LTR, 3′ LTR, and/or psi packaging element. In specific cases, the psi packaging is incorporated between the 5′ LTR and the antigen receptor coding sequence. The vector may or may not comprise pUC19 sequence. In some aspects of the vector, at least one cistron encodes for a cytokine (interleukin 15 (IL-15), IL-7, IL-21, IL-18, IL-12, or IL-2, for example), chemokine, cytokine receptor, and/or homing receptor.

When 2A cleavages sites are utilized in the vector, the 2A cleavage site may comprise a P2A, T2A, E2A and/or F2A site.

In addition to one cistron encoding a CD70-targeting CAR, any cistron of the vector may comprise a suicide gene. Any cistron of the vector may encode a reporter gene. In specific embodiments, a first cistron encodes a suicide gene, a second cistron encodes a CD70-targeting CAR, a third cistron encodes a reporter gene, and a fourth cistron encodes a cytokine. In certain embodiments, a first cistron encodes a suicide gene, a second cistron encodes a a CD70-targeting CAR, a third cistron encodes a second CAR or another antigen receptor, and a fourth cistron encodes a cytokine. In specific embodiments, different parts of the a CD70-targeting CAR and/or another receptor are encoded by corresponding modular components and a first component of the second cistron encodes an antigen binding domain, a second component encodes a hinge and/or transmembrane domain, a third component encodes a costimulatory domain, and a fourth component encodes a signaling domain.

In specific embodiments, at least one of the cistrons encodes a suicide gene. In some embodiments, at least one of the cistrons encodes a cytokine. In certain embodiments, at least one cistron encodes a CD70-targeting CAR. A cistron may or may not encode a reporter gene. In certain embodiments, at least two cistrons encode two different antigen receptors (e.g., CARs and/or TCRs). A cistron may or may not encode a reporter gene.

In particular configurations of the genetic cargo of interest, a single vector may comprise a cistron that encodes a CD70-targeting CAR and a cistron that encodes a second antigen receptor that is non-identical to the CD70-targeting receptor. In specific embodiments, the first antigen receptor encodes a a CD70-targeting CAR and the second antigen receptor encodes a TCR, or vice versa. In particular embodiments, a vector comprising separate cistrons that respectively encode a CD70-targeting CAR and a second antigen receptor also comprises a third cistron that encodes a cytokine or chemokine and a fourth cistron that encodes a suicide gene. However, the suicide gene and/or the cytokine (or chemokine) may not be present on the vector.

In particular embodiments, at least one cistron comprises multiple component(s) themselves that are modular. For example, one cistron may encode a multi-component gene product, such as an antigen receptor having multiple parts; in specific cases the antigen receptor is encoded from a single cistron, thereby ultimately producing a single polypeptide. The cistron encoding multiple components may have the multiple components separated by 1, 2, 3, 4, 5, or more restriction enzyme digestion sites, including 1, 2, 3, 4, 5, or more restriction enzyme digestion sites that are unique to the vector comprising the cistron (FIGS. 1A and 1B). In specific embodiments, a cistron having multiple components encodes an antigen receptor having multiple corresponding parts each attributing a unique function to the receptor. In a specific embodiment, each or the majority of components of the multi-component cistrons is separated by one or more restriction enzyme digestion sites that are unique to the vector, allowing the interchangeability of separate components when desired.

In specific embodiments, each component of a multi-component cistron corresponds to a different part of an encoded antigen receptor, such as a CD70-targeting CAR. In illustrative embodiments, component 1 may encode a CD70 antigen-binding domain of the receptor; component 2 may encode a hinge domain of the receptor; component 3 may encode a transmembrane domain of the receptor; component 4 may encode a costimulatory domain of the receptor, and component 5 may encode a signaling domain of the receptor. In specific embodiments, a CD70-targeting CAR may comprise one or more costimulatory domains, each separated by unique restriction enzyme digestion sites for interchangeability of the costimulatory domain(s) within the receptor.

In specific embodiments, there is a polycistronic vector having four separate cistrons where adjacent cistrons are separated by a 2A cleavage site, although in specific embodiments instead of a 2A cleavage site there is an element that directly or indirectly causes separate polypeptides to be produced from the cistrons (such as an IRES sequence). For example, four separate cistrons may be separated by three 2A peptide cleavage sites, and each cistron has restriction sites (X₁, X₂, etc.) flanking each end of the cistron to allow for interchangeability of the particular cistron, such as with another cistron or other type of sequence, and upon using standard recombination techniques. In specific embodiments, the restriction enzyme site(s) that flank each of the cistrons is unique to the vector to allow ease of recombination, although in alternative embodiments the restriction enzyme site is not unique to the vector.

In particular embodiments, the vector provides for a unique, second level of modularity by allowing for interchangeability within a particular cistron, including within multiple components of a particular cistron. The multiple components of a particular cistron may be separated by one or more restriction enzyme sites, including those unique to the vector, to allow for interchangeability of one or more components within the cistron. As an example, cistron 2 may comprise five separate components, although there may be 2, 3, 4, 5, 6, or more components per cistron. As an example, a vector may include cistron 2 that has five components each separated by unique enzyme restriction sites X₉, X₁₀, X₁₁, X₁₂, X₁₃, and X₁₄, to allow for standard recombination to exchange different components 1, 2, 3, 4, and/or 5. In some cases, there may be multiple restriction enzyme sites between the different components (that are unique, although alternatively one or more are not unique) and there may be sequence in between the multiple restriction enzyme sites (although alternatively there may not be). In certain embodiments, all components encoded by a cistron are designed for the purpose of being interchangeable. In particular cases, one or more components of a cistron are designed to be interchangeable, whereas one or more other components of the cistron may not be designed to be interchangeable.

In specific embodiments, a cistron encodes a CD70-targeting CAR molecule having multiple components. For example, cistron 2 may be comprised of sequence that encodes a CD70-targeting CAR molecule having its separate components represented by component 1, component 2, component 3, etc. The CAR molecule may comprise 2, 3, 4, 5, 6, 7, 8, or more interchangeable components. In a specific example, component 1 encodes a CD70 scFv; component 2 encodes a hinge; component 3 encodes a transmembrane domain; component 4 encodes a costimulatory domain (although there may also be component 4′ that encodes a second or more costimulatory domain flanked by restriction sites for exchange); and component 5 encodes a signaling domain. In a particular example, component 1 encodes a CD70 scFv; component 2 encodes a IgG1 hinge and/or transmembrane domain; component 3 encodes CD28; and component 4 encodes CD3 zeta.

One of skill in the art recognizes in the design of the vector that the various cistrons and components must be configured such that they are kept in frame when necessary.

In a particular example, cistron 1 encodes a suicide gene; cistron 2 encodes a CD70-targeting CAR; cistron 3 encodes a reporter gene; cistron 4 encodes a cytokine; component 1 of cistron 2 encodes a CD70 scFv; component 2 of cistron 2 encodes IgG1 hinge; component 3 of cistron 2 encodes CD28; and component 4 encodes CD3 zeta.

A restriction enzyme site may be of any kind and may include any number of bases in its recognition site, such as between 4 and 8 bases; the number of bases in the recognition site may be at least 4, 5, 6, 7, 8, or more. The site when cut may produce a blunt cut or sticky ends. The restriction enzyme may be of Type I, Type II, Type III, or Type IV, for example. Restriction enzyme sites may be obtained from available databases, such as Integrated relational Enzyme database (IntEnz) or BRENDA (The Comprehensive Enzyme Information System).

Exemplary vectors may be circular and by convention, where position 1 (12 o'clock position at the top of the circle, with the rest of the sequence in clock-wise direction) is set at the start of 5′ LTR.

In embodiments wherein self-cleaving 2A peptides are utilized, the 2A peptides may be 18-22 amino-acid (aa)-long viral oligopeptides that mediate “cleavage” of polypeptides during translation in eukaryotic cells. The designation “2A” refers to a specific region of the viral genome and different viral 2As have generally been named after the virus they were derived from. The first discovered 2A was F2A (foot-and-mouth disease virus), after which E2A (equine rhinitis A virus), P2A (porcine teschovirus-1 2A), and T2A (thosea asigna virus 2A) were also identified. The mechanism of 2A-mediated “self-cleavage” was discovered to be ribosome skipping the formation of a glycyl-prolyl peptide bond at the C-terminus of the 2A.

In specific cases, the vector may be a γ-retroviral transfer vector. The retroviral transfer vector may comprises a backbone based on a plasmid, such as the pUC19 plasmid (large fragment (2.63 kb) in between HindIII and EcoRI restriction enzyme sites). The backbone may carry viral components from Moloney Murine Leukemia Virus (MoMLV) including 5′ LTR, psi packaging sequence, and 3′ LTR. LTRs are long terminal repeats found on either side of a retroviral provirus, and in the case of a transfer vector, brackets the genetic cargo of interest, such as CD70-targeting CARs and associated components. The psi packaging sequence, which is a target site for packaging by nucleocapsid, is also incorporated in cis, sandwiched between the 5′ LTR and the CAR coding sequence. Thus, the basic structure of an example of a transfer vector can be configured as such: pUC19 sequence—5′ LTR—psi packaging sequence—genetic cargo of interest—3′ LTR—pUC19 sequence. This system may also be applied to other viral and non-viral vectors, including but not limited lentivirus, adenovirus AAV, as well as non-viral plasmids.

### V. CELLS

The present disclosure encompasses immune cells or stem cells of any kind that harbor at least one vector that encodes a CD70-targeting receptor and that also may encode at least one cytokine and/or at least one suicide gene. In some cases, different vectors encode the CAR vs. encodes the suicide gene and/or cytokine. The immune cells, including NK cells, may be derived from cord blood, peripheral blood, induced pluripotent stem cells (iPSCs), hematopoietic stem cells (HSCs), bone marrow, or a mixture thereof. The NK cells may be derived from a cell line such as, but not limited to, NK-92 cells, for example. The NK cell may be a cord blood mononuclear cell, such as a CD56+NK cell.

The present disclosure encompasses immune or other cells of any kind, including conventional T cells, gamma-delta T cells, NKT and invariant NK T cells, regulatory T cells, macrophages, B cells, dendritic cells, mesenchymal stromal cells (MSCs), or a mixture thereof.

In some cases, the cells have been expanded in the presence of an effective amount of universal antigen presenting cells (UAPCs), including in any suitable ratio. The cells may be cultured with the UAPCs at a ratio of 10:1 to 1:10; 9:1 to 1:9; 8:1 to 1:8; 7:1 to 1:7; 6:1 to 1:6; 5:1 to 1:5; 4:1 to 1:4; 3:1 to 1:3; 2:1 to 1:2; or 1:1, including at a ratio of 1:2, for example. In some cases, the NK cells were expanded in the presence of IL-2, such as at a concentration of 10-500, 10-400, 10-300, 10-200, 10-100, 10-50, 100-500, 100-400, 100-300, 100-200, 200-500, 200-400, 200-300, 300-500, 300-400, or 400-500 U/mL.

Following genetic modification with the vector(s), the NK cells may be immediately infused or may be stored. In certain aspects, following genetic modification, the cells may be propagated for days, weeks, or months ex vivo as a bulk population within about 1, 2, 3, 4, 5 days or more following gene transfer into cells. In a further aspect, the transfectants are cloned and a clone demonstrating presence of a single integrated or episomally maintained expression cassette or plasmid, and expression of the CD70-targeting CAR is expanded ex vivo. The clone selected for expansion demonstrates the capacity to specifically recognize and lyse CD70-expressing target cells. The recombinant immune cells may be expanded by stimulation with IL-2, or other cytokines that bind the common gamma-chain (e.g., IL-7, IL-12, IL-15, IL-21, and others). The recombinant immune cells may be expanded by stimulation with artificial antigen presenting cells. In a further aspect, the genetically modified cells may be cryopreserved.

Embodiments of the disclosure encompass cells that express one or more CD70-targeting CARs and one or more suicide genes as encompassed herein. The NK cell comprises a recombinant nucleic acid that encodes one or more CD70-targeting CARs and one or more engineered nonsecretable, membrane bound TNF-alpha mutant polypeptides, in specific embodiments. In specific embodiments, in addition to expressing one or more CD70-targeting CARs and TNF-alpha mutant polypeptides, the cell also comprises a nucleic acid that encodes one or more therapeutic gene products.

The cells may be obtained from an individual directly or may be obtained from a depository or other storage facility. The cells as therapy may be autologous or allogeneic with respect to the individual to which the cells are provided as therapy.

The cells may be from an individual in need of therapy for a medical condition, and following their manipulation to express the CD70-targeting CAR, optional suicide gene, optional cytokine(s), and optional therapeutic gene product(s) (using standard techniques for transduction and expansion for adoptive cell therapy, for example), they may be provided back to the individual from which they were originally sourced. In some cases, the cells are stored for later use for the individual or another individual.

The immune cells may be comprised in a population of cells, and that population may have a majority that are transduced with one or more CD70-targeting receptors and/or one or more suicide genes and/or one or more cytokines. A cell population may comprise 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% of immune cells that are transduced with one or more CD70-targeting receptors and/or one or more suicide genes and/or one or more cytokines. The one or more CD70-targeting receptors and/or one or more suicide genes and/or one or more cytokines may be separate polypeptides.

The immune cells may be produced with the one or more CD70-targeting receptors and/or one or more suicide genes and/or one or more cytokines for the intent of being modular with respect to a specific purpose. For example, cells may be generated, including for commercial distribution, expressing a CD70-targeting CARs and/or one or more suicide genes and/or one or more cytokines (or distributed with a nucleic acid that encodes the mutant for subsequent transduction), and a user may modify them to express one or more other genes of interest (including therapeutic genes) dependent upon their intended purpose(s). For instance, an individual interested in treating CD70-positive cells, including CD70-positive cancer, may obtain or generate suicide gene-expressing cells (or heterologous cytokine-expressing cells) and modify them to express a receptor comprising a CD70-specific scFv, or vice versa.

In particular embodiments, NK cells are utilized, and the genome of the transduced NK cells expressing the one or more CD70-targeting CARs and/or one or more suicide genes and/or one or more cytokines may be modified. The genome may be modified in any manner, but in specific embodiments the genome is modified by CRISPR gene editing, for example. The genome of the cells may be modified to enhance effectiveness of the cells for any purpose.

### VI. GENE EDITING OF CD70-SPECIFIC CAR CELLS

In particular embodiments, cells comprising at least a CD70-specific engineered receptor are gene edited to modify expression of one or more endogenous genes in the cell. In specific cases, the CD70-specific CAR cells are modified to have reduced levels of expression of one or more endogenous genes, including inhibition of expression of one or more endogenous genes (that may be referred to as knocked out). Such cells may or may not be expanded.

In particular cases, one or more endogenous genes of the CD70-specific CAR cells are modified, such as disrupted in expression where the expression is reduced in part or in full. In specific cases, one or more genes are knocked down or knocked out using processes of the disclosure. In specific cases, multiple genes are knocked down or knocked out, and this may or may not occur in the same step in their production. The genes that are edited in the CD70-specific CAR cells may be of any kind, but in specific embodiments the genes are genes whose gene products inhibit activity and/or proliferation of the CD70-specific CAR cells, including CD70-specific CAR NK cells, such as those derived from cord blood, as one example. In specific cases the genes that are edited in the CD70-specific CAR cells allow the CD70-specific CAR cells to work more effectively in a tumor microenvironment. In specific cases, the genes are one or more of NKG2A, SIGLEC-7, LAGS, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, CD5, and CD7. In specific embodiments, the TGFBR2 gene is knocked out or knocked down in the CD70-specific CAR cells.

In some embodiments, the gene editing is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins; in some embodiments, CpF1 is utilized instead of Cas9. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions or alterations as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). In some cases, CpF1 may be used as an endonuclease instead of Cas9. The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5- transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

### VII. METHODS OF TREATMENT

In various embodiments, cells expressing endogenous CD70 on their surface are targeted for the purpose of improving a medical condition in an individual that has the medical condition or for the purpose of reducing the risk or delaying the severity and/or onset of the medical condition in an individual. In specific cases, cancer cells expressing endogenous CD70 are targeted for the purpose of killing the cancer cells. In other cases, CD70 is targeted as CD70-positive cells, but the CD70-positive cells are not cancer cells. In such cases, the CD70-positive cells may be immunoregulatory cells, such as T regulatory cells. Targeting and depleting CD70+ regulatory T cells can further enhance immunotherapy of cancer by removing the immunosuppressive effect of this cell subset. Thus, in specific embodiments, there are methods of reducing immunosuppression of cancer therapy by providing an effective amount of cells that target CD70, as described herein.

CD70-targeting CAR constructs, nucleic acid sequences, vectors, immune cells and so forth as contemplated herein, and/or pharmaceutical compositions comprising the same, are used for the prevention, treatment or amelioration of a cancerous disease, such as a tumorous disease. In particular embodiments, the pharmaceutical composition of the present disclosure may be particularly useful in preventing, ameliorating and/or treating cancer, including cancers that express CD70 and that may or may not be solid tumors, for example.

The immune cells for which the CD70-targeting receptor is utilized may be NK, T cells, gamma delta T cells, or NKT or invariant NKT (iNKT), or induced NKT cells engineered for cell therapy for mammals, in particular embodiments. In such cases where the cells are NK cells, the NK cell therapy may be of any kind and the NK cells may be of any kind. In specific embodiments, the cells are NK cells that have been engineered to express one or more CD70-targeting CARs and/or one or more suicide genes and/or one or more cytokines. In specific embodiments, the cells are NK cells that are transduced with a CD70-targeting CAR.

In particular embodiments, the present disclosure contemplates, in part, CD70 CAR-expressing cells, CD70-targeting CAR constructs, CD70-targeting CAR nucleic acid molecules and CD70-targeting CAR vectors that can be administered either alone or in any combination using standard vectors and/or gene delivery systems, and in at least some aspects, together with a pharmaceutically acceptable carrier or excipient. In certain embodiments, subsequent to administration, the nucleic acid molecules or vectors may be stably integrated into the genome of the subject.

In specific embodiments, viral vectors may be used that are specific for certain cells or tissues and persist in NK cells. Suitable pharmaceutical carriers and excipients are well known in the art. The compositions prepared according to the disclosure can be used for the prevention or treatment or delaying the above identified diseases.

Furthermore, the disclosure relates to a method for the prevention, treatment or amelioration of a tumorous disease comprising the step of administering to a subject in the need thereof an effective amount of cells that express a CD70-targeting CAR, a nucleic acid sequence, a vector, as contemplated herein and/or produced by a process as contemplated herein.

Possible indications for administration of the composition(s) of the exemplary CD70-targeting CAR cells are cancerous diseases, including tumorous diseases, including B cell malignancies, multiple myeloma, breast cancer, glioblastoma, renal cancer, pancreatic cancer, or lung cancer, for example. Exemplary indications for administration of the composition(s) of CD70-targeting CAR cells are cancerous diseases, including any malignancies that express CD70. The administration of the composition(s) of the disclosure is useful for all stages (I, II, III, or IV) and types of cancer, including for minimal residual disease, early cancer, advanced cancer, and/or metastatic cancer and/or refractory cancer, for example.

The disclosure further encompasses co-administration protocols with other compounds, e.g. bispecific antibody constructs, targeted toxins or other compounds, which act via immune cells. The clinical regimen for co-administration of the inventive compound(s) may encompass co-administration at the same time, before or after the administration of the other component. Particular combination therapies include chemotherapy, radiation, surgery, hormone therapy, or other types of immunotherapy.

Embodiments relate to a kit comprising a CD70-targeting CAR construct as defined herein, a nucleic acid sequence as defined herein, a vector as defined herein and/or a host cell (such as an immune cell) as defined herein. It is also contemplated that the kit of this disclosure comprises a pharmaceutical composition as described herein above, either alone or in combination with further medicaments to be administered to an individual in need of medical treatment or intervention.

A. Pharmaceutical Compositions

Also provided herein are pharmaceutical compositions and formulations comprising transduced NK cells and a pharmaceutically acceptable carrier. The transduced cells may be comprised in a media suitable for transfer to an individual and/or media suitable for preservation, such as cryopreservation, including prior to transfer to an individual.

Pharmaceutical compositions and formulations as described herein can be prepared by mixing the active ingredients (such as the cells) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn— protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

B. Combination Therapies

In certain embodiments, the compositions and methods of the present embodiments involve an immune cell population (including NK cell population) in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, hormone therapy, oncolytic viruses, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An immune cell therapy may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an immune cell therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or cell therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclophosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine,plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody—drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p9′7), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons □, □□ and □, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VIII. KITS OF THE DISCLOSURE

Any of the compositions described herein may be comprised in a kit. In a non-limiting example, cells, reagents to produce cells, vectors, and reagents to produce vectors and/or components thereof may be comprised in a kit. In certain embodiments, NK cells may be comprised in a kit, and they may or may not yet express a CD70-targeting receptor, an optional cytokine, or an optional suicide gene. Such a kit may or may not have one or more reagents for manipulation of cells. Such reagents include small molecules, proteins, nucleic acids, antibodies, buffers, primers, nucleotides, salts, and/or a combination thereof, for example. Nucleotides that encode one or more CD70-targeting CARs, suicide gene products, and/or cytokines may be included in the kit. Proteins, such as cytokines or antibodies, including monoclonal antibodies, may be included in the kit. Nucleotides that encode components of engineered CAR receptors may be included in the kit, including reagents to generate same.

In particular aspects, the kit comprises the NK cell therapy of the disclosure and also another cancer therapy. In some cases, the kit, in addition to the cell therapy embodiments, also includes a second cancer therapy, such as chemotherapy, hormone therapy, and/or immunotherapy, for example. The kit(s) may be tailored to a particular cancer for an individual and comprise respective second cancer therapies for the individual.

The kits may comprise suitably aliquoted compositions of the present disclosure. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also may generally contain a second, third or other additional container into which the additional components may be separately placed. However, various combinations of components may be comprised in a vial. The kits of the present invention also will typically include a means for containing the composition and any other reagent containers in close confinement for commercial sale. Such containers may include injection or blow-molded plastic containers into which the desired vials are retained.

### IX. EXAMPLES

The following examples are included to demonstrate certain non-limiting aspects of the disclosure. It should be appreciated by those of skill in the art that the techniques disclosed in the examples that follow represent techniques discovered by the inventors to function well in the practice of the disclosed subject matter. However, those of skill in the art should, in light of the present disclosure, appreciate that many changes can be made in the specific embodiments that are disclosed and still obtain a like or similar result without departing from the spirit and scope of the disclosed subject matter.

### Example 1

### CAR.CD70 NK Cells to Target AML

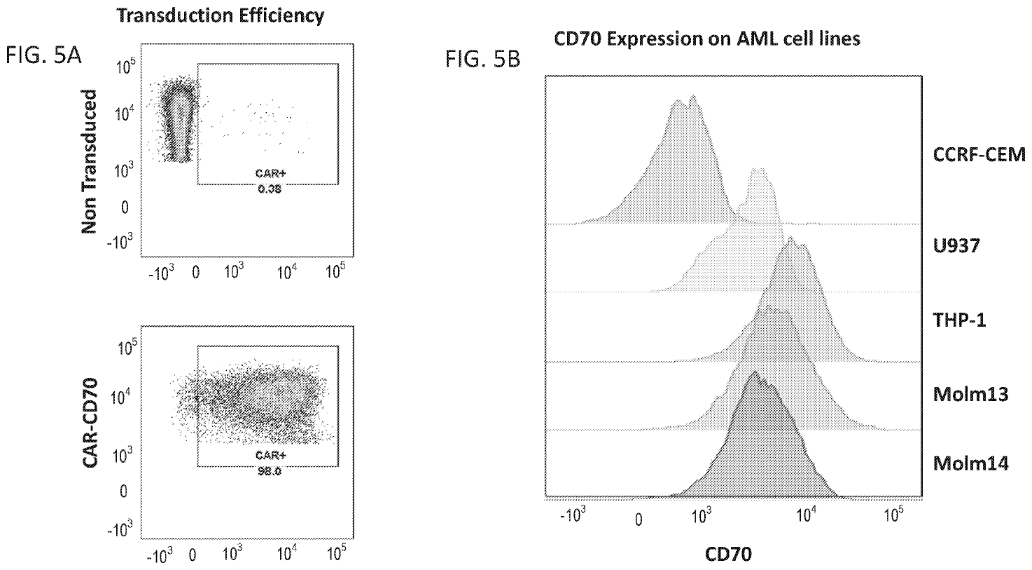

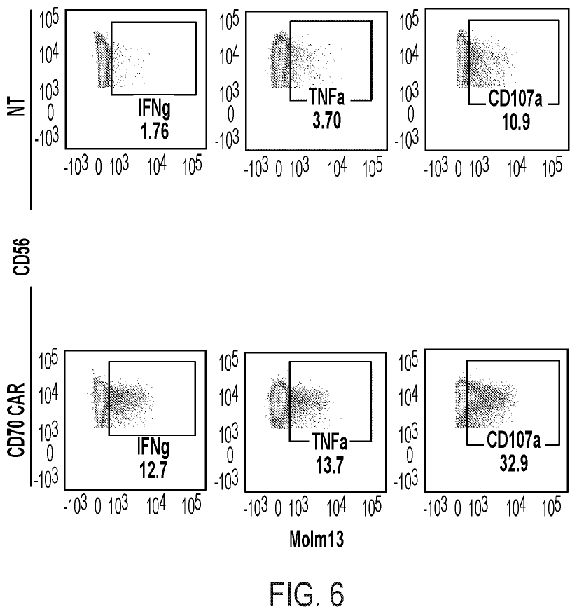

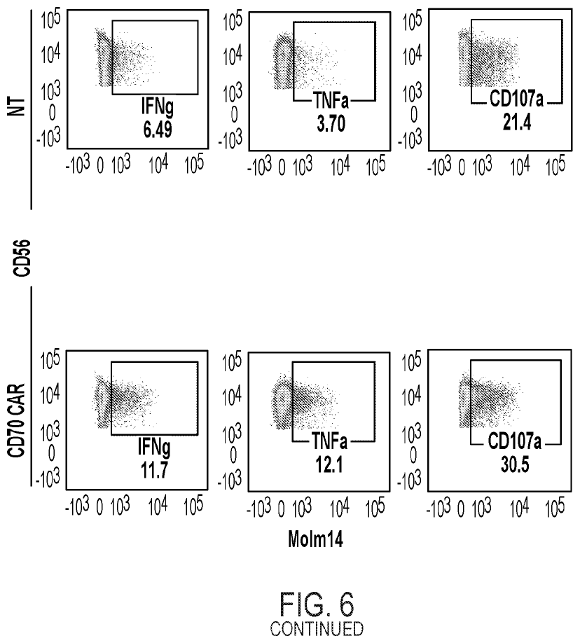

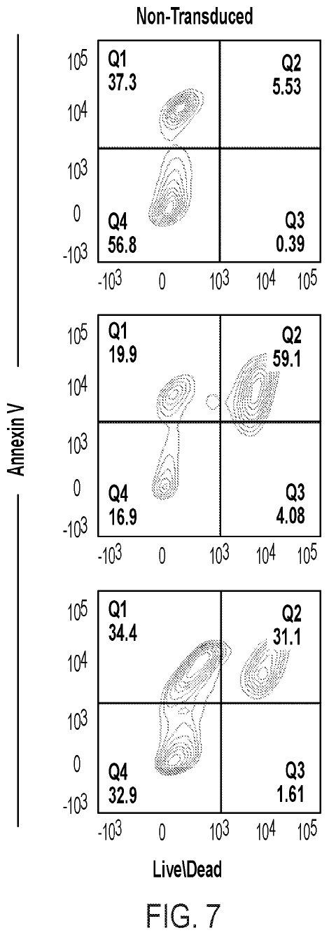

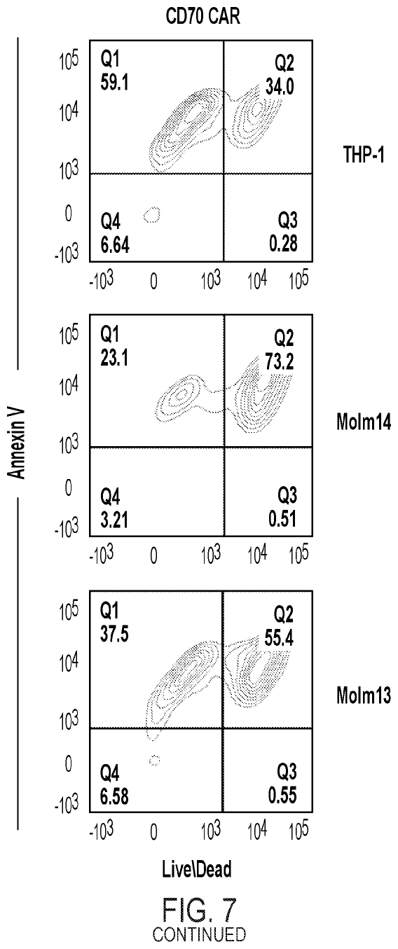

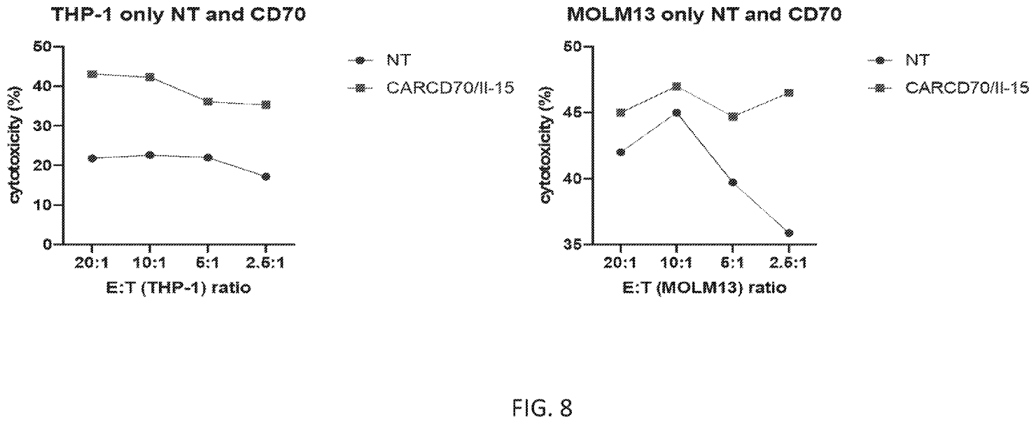

In particular embodiments, CD70-specific CAR NK cells are utilized to target acute myeloid leukemia (AML). FIG. 5A demonstrates transduction efficiency in CAR-CD70 NK cells, as compared to non-transduced cells. FIG. 5B demonstrates CD70 expression on a variety of AML cell lines. For Molm13 and Molm14 AML cell lines, FIG. 6 demonstrates a functional assay for activity of CD70 CAR in CD70 CAR/IL-15-expressing NK cells, versus non-transduced cells. Annexin V assays demonstrated enhanced killing of difference AML cells lines compared to non-transduced cells (FIG. 7). Chromium release assays also demonstrated greater killing of AML cell lines using CD70 CAR-expressing NK cells that also expressed IL-15.

### Example 2

### CAR.CD70 NK Cells to Target Lung Cancer

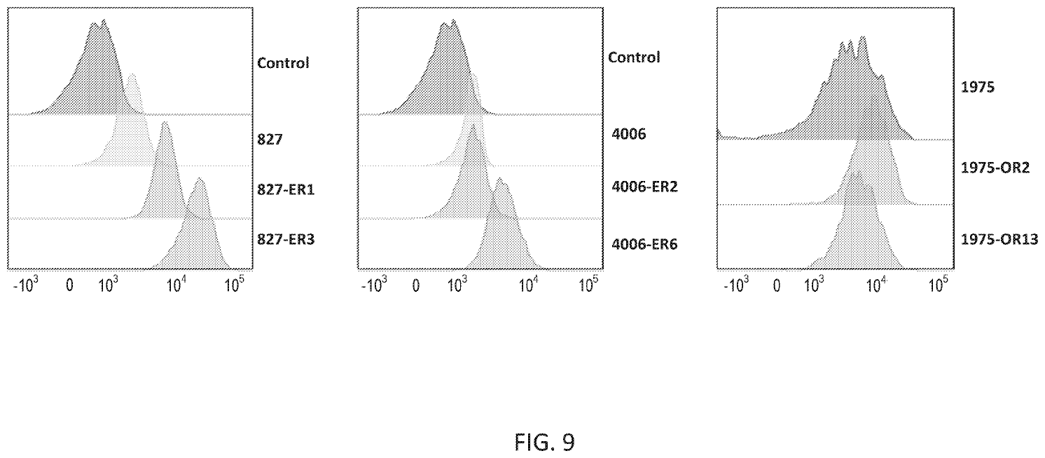

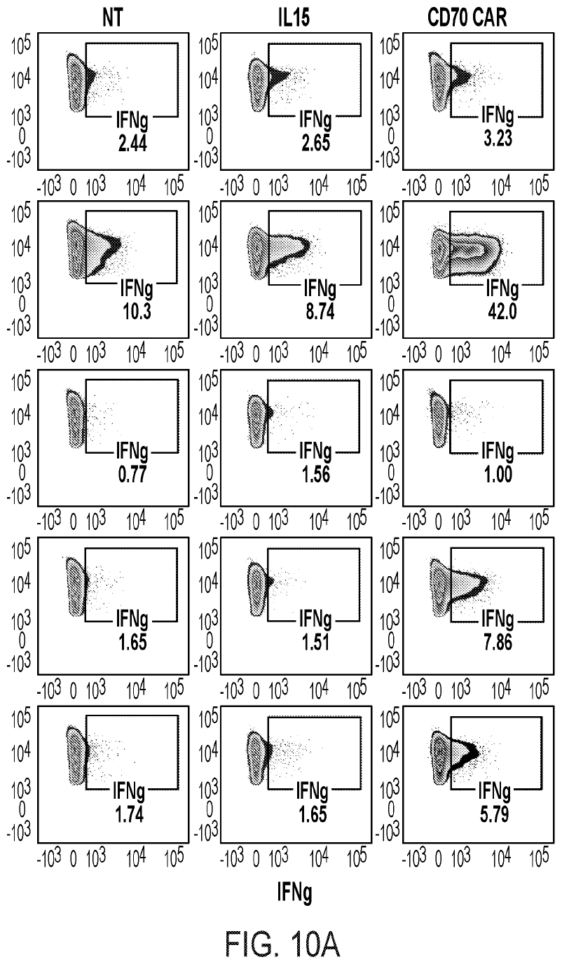

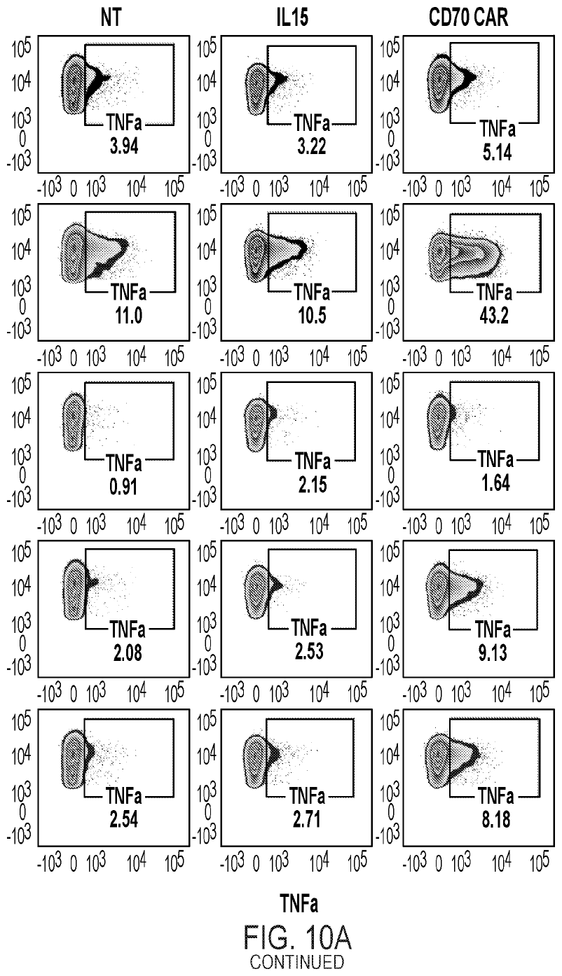

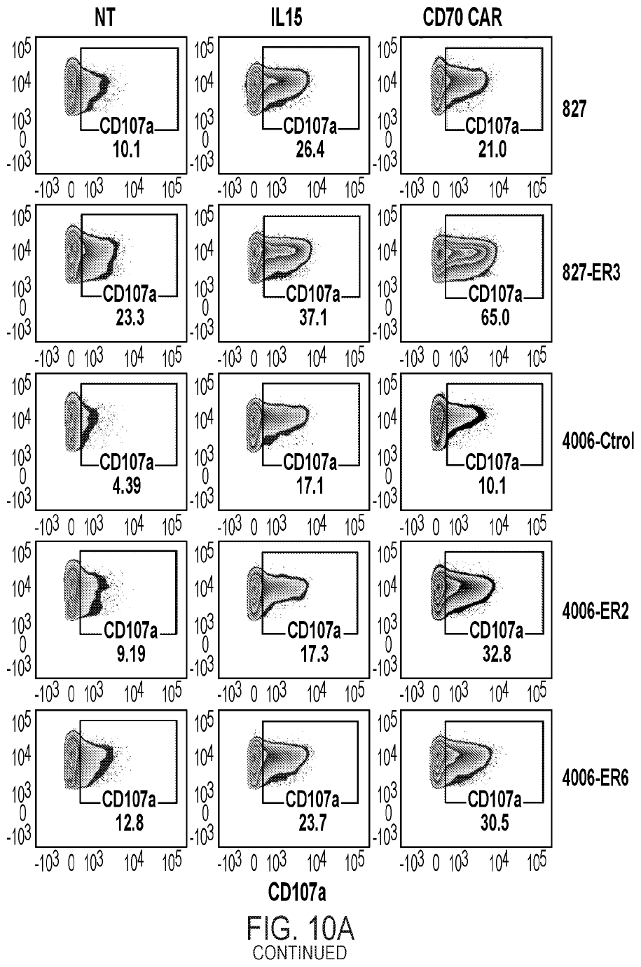

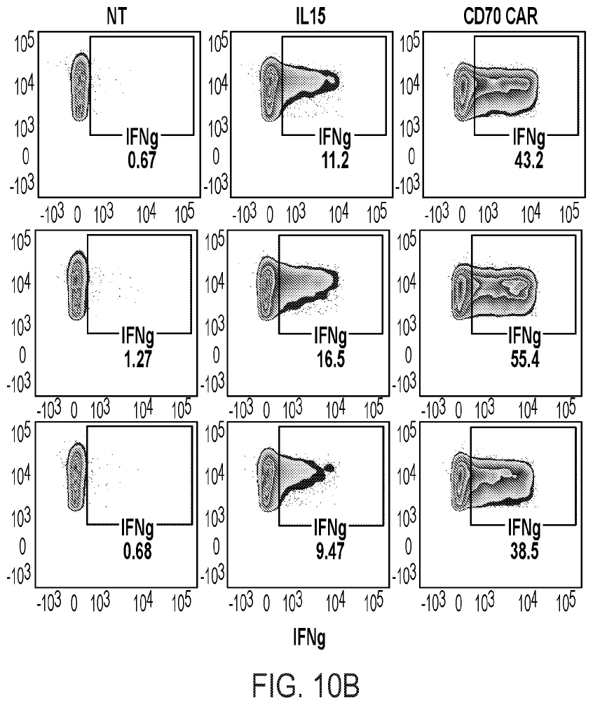

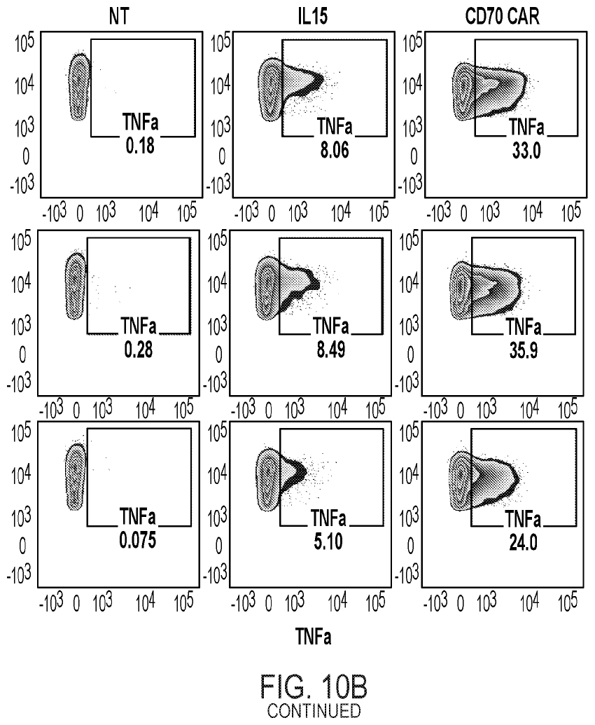

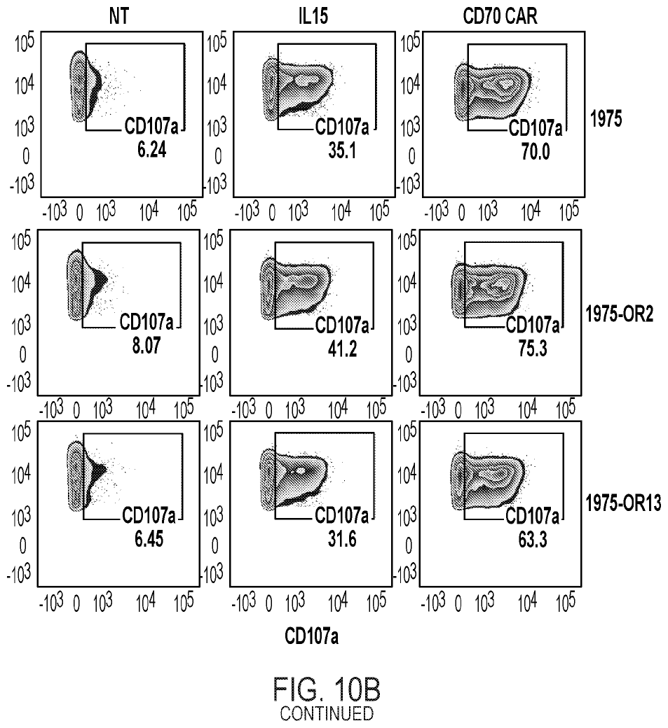

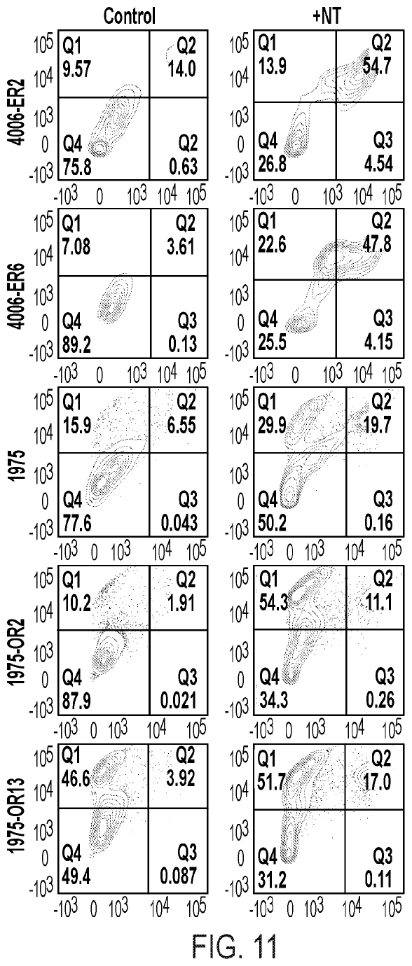

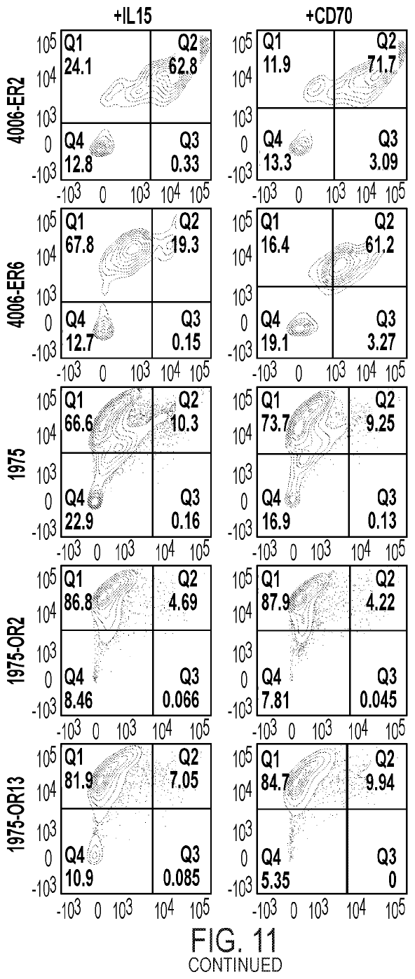

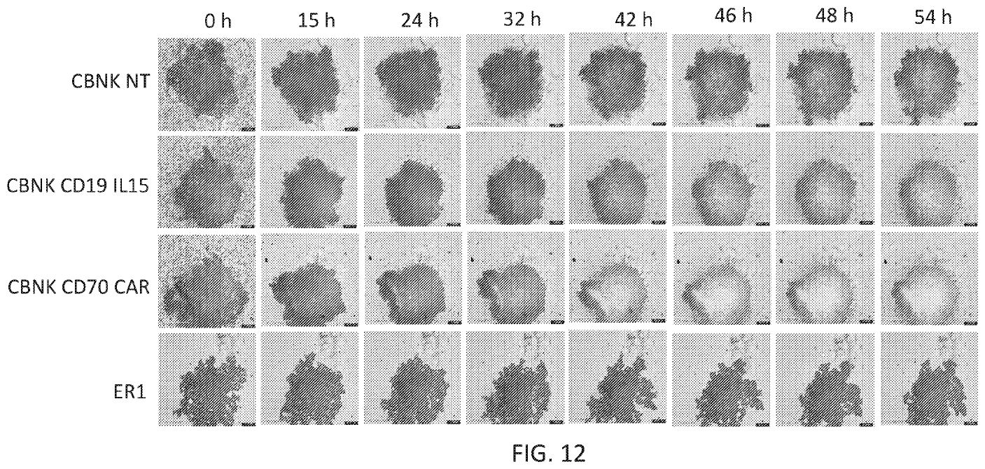

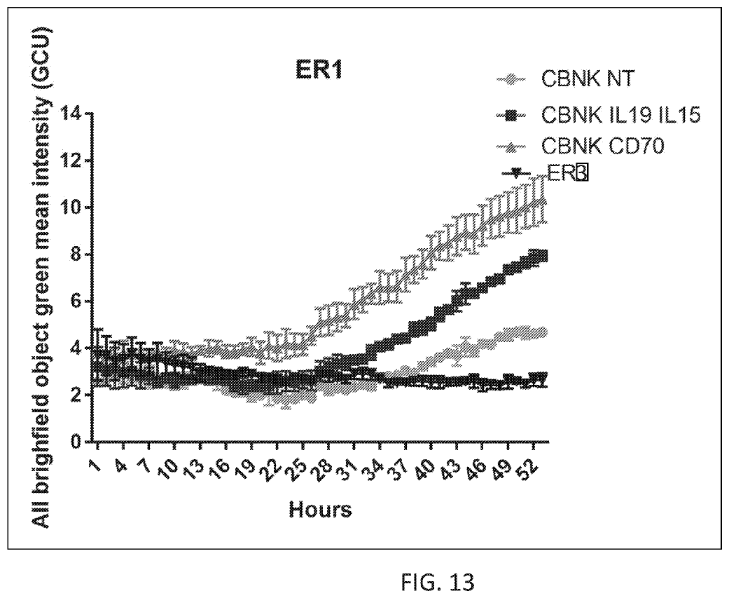

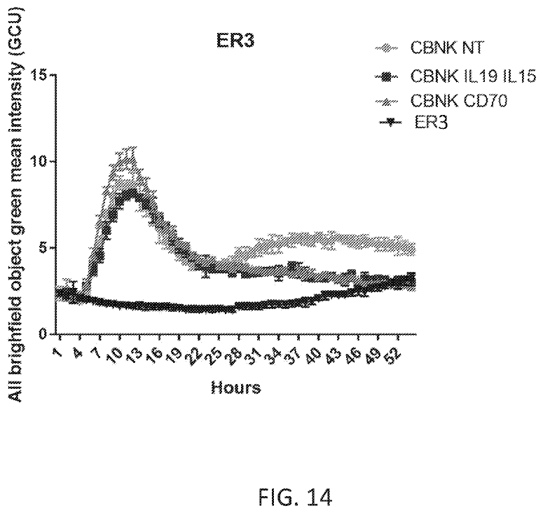

In some embodiments, the reagents are utilized to target and kill CD70-expressing lung cancer, as one example of a solid tumor. FIG. 9 demonstrates CD70 expression on a variety of lung cancer cell lines. Employing CD70 CAR-expressing NK cells resulted in greater toxicity against a variety of lung cancer cell lines when compared to non-transduced cells and IL-15 transduced NK cells (FIGS. 10A and 10B). Annexin staining demonstrated greater toxicity in a variety of lung cancer cell lines when comparing CD70 CAR-expressing NK cells as compared to non-transduced cells and IL-15 transduced NK cells (FIG. 11). In FIG. 12, as assessed by caspase expression in lung cancer cell line spheroids, CD70 CAR-expressing NK cells demonstrated greater toxicity than non-transduced NK cells or NK cells transduced with IL-15 alone (no CAR). Using an Incucyte® assay, compared to non-transduced (NT) and IL-15 transduced NK cells, CD70 CAR/IL-15-expressing NK cells exert greater cytotoxicity against an ER1 lung cancer cell line (FIG. 13). Using an Incucyte® assay, compared to non-transduced (NT) and IL-15 transduced NK cells, CD70 CAR/IL-15-expressing NK cells exert greater cytotoxicity against an ER3 lung cancer cell line (FIG. 14).

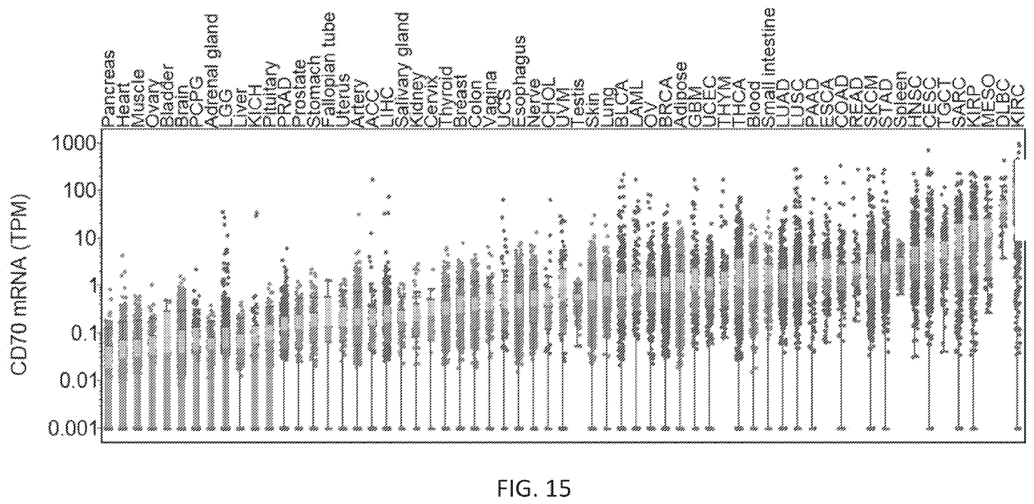

Other CD70-positive cancers than lung cancer may be treated with methods and compositions of the disclosure (see FIG. 15 for examples).

### Example 3

### Cord Blood-Derived Natural Killer (CBNK) Cells Transduced with CD70 CAR Against a Variety of Cancers

Acute Myeloid Leukemia (AML)

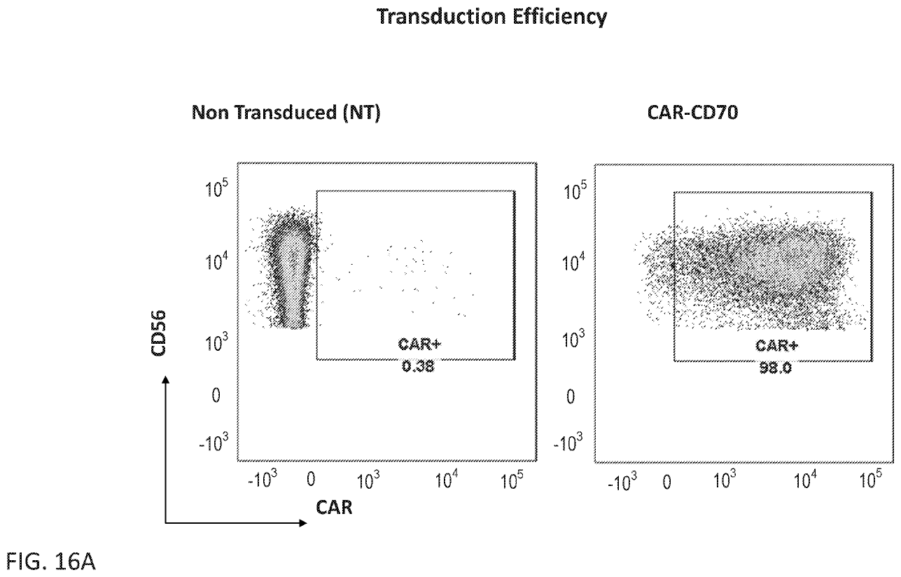

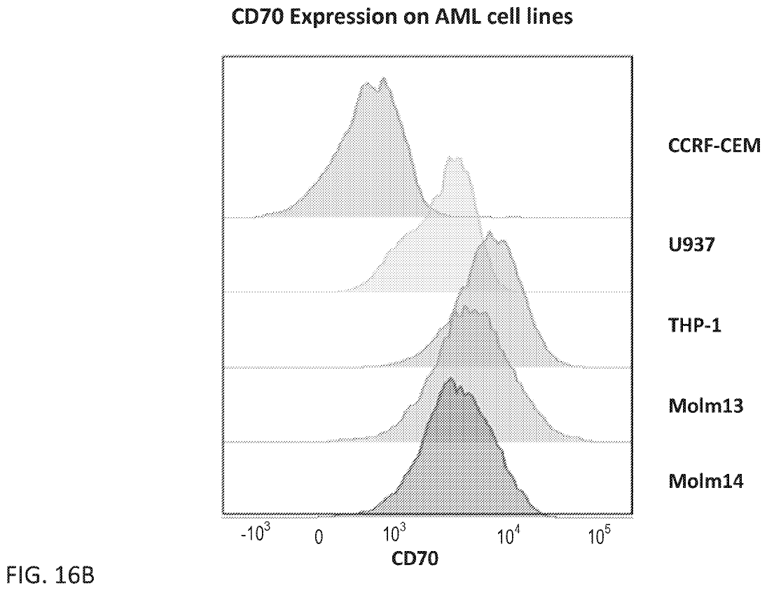

FIGS. 16A-16B show CD70 CAR transduction efficiency in CBNK cells and expression of CD70 in various acute myeloid leukemia (AML) targets. FIG. 16A shows that CD70 CAR was successfully transduced in CBNK cells with transduction efficiency of 98% when compared to non-transduced cells. FIG. 16B demonstrates that CD70 was expressed in surface of various AML targets.

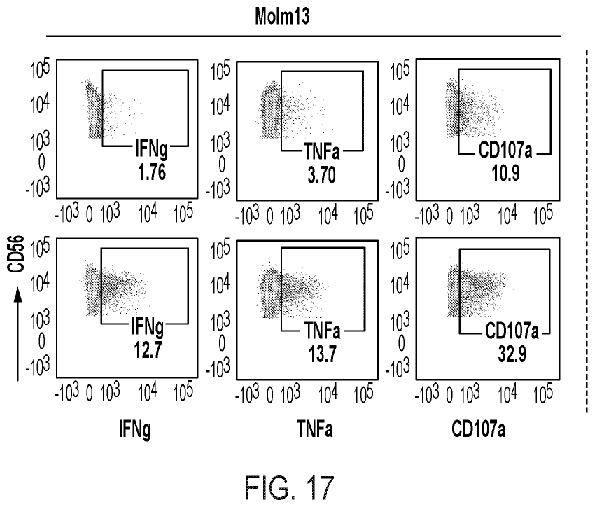

FIG. 17 shows expression of intracellular cytokines and degranulation marker expression in CBNK CD70 CAR cells when co-cultured with Molm13 and Molm14 cells. Compared to non-transduced (NT) cells, CBNK cells transduced with CD70 CAR showed increased cytokines (interferon gamma and tumor necrosis factor alpha) secretion and degranulation marker CD107a expression when co-cultured with Molm13 (left) and Molm14 (right), suggesting enhanced cytotoxic activity against CD70 expressing AML cells.

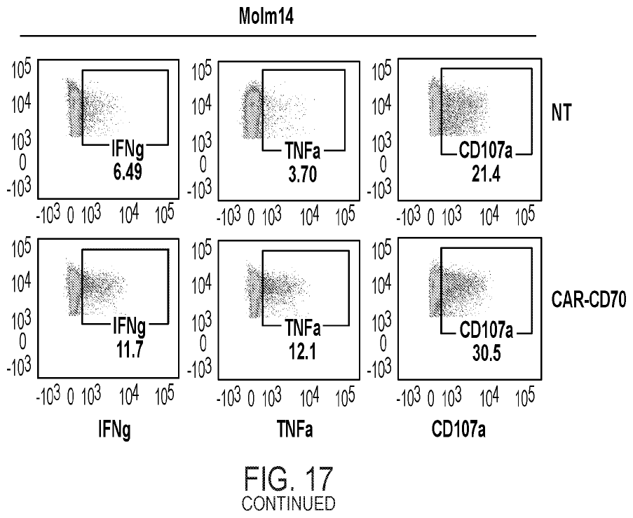

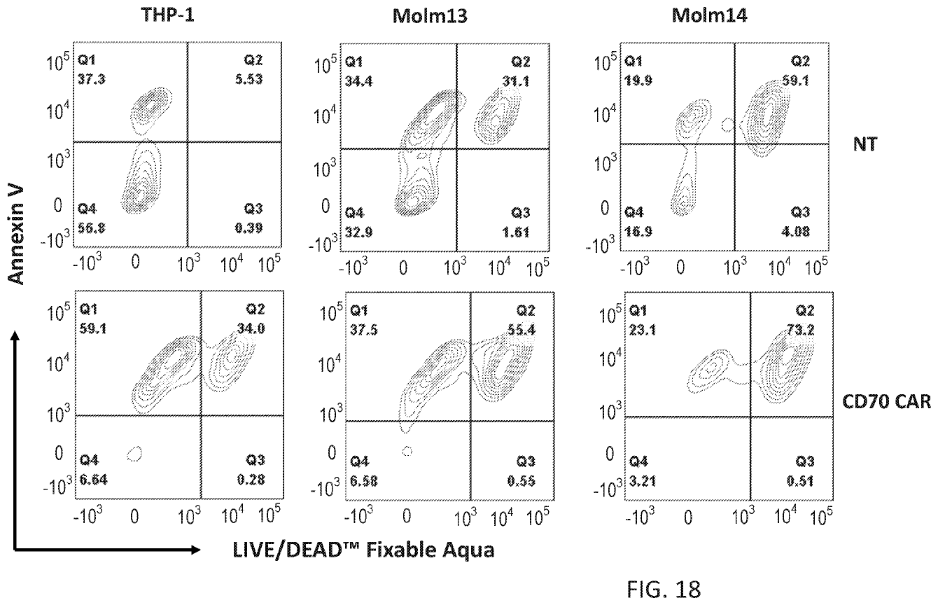

FIG. 18 shows Annexin V staining to assess the apoptosis of AML target cells after co-culture with CBNK CD70 CAR cells. Compared to non-transduced (NT) cells, CBNK cells transduced with CD70 CAR showed increased apoptosis of THP-1, Molm13 and Molm14 cells, as shown by Annexin V-LIVE/DEAD™ Fixable Aqua staining assay, suggesting the enhanced cytotoxic activity of CBNK cells transduced with CD70 CAR against AML cells.

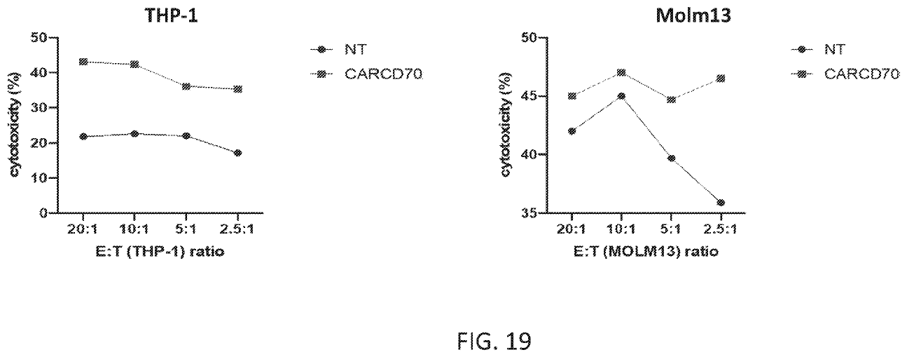

FIG. 19 demonstrates a chromium release assay to assess the cytotoxic activity of CBNK CD70 CAR against AML target cells. Compared to non-transduced (NT) cells, CBNK cells transduced with CD70 CAR showed increased cytotoxicity of THP-1 (left) and Molm13 (right) cells, as shown by chromium release assay, suggesting that CBNK CD70 CAR cells have greater killing activity against AML cells.

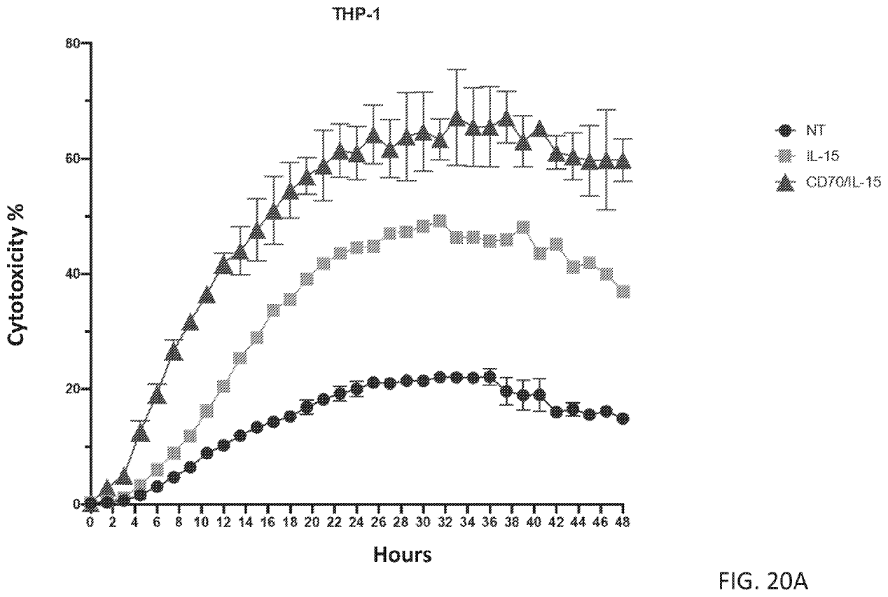

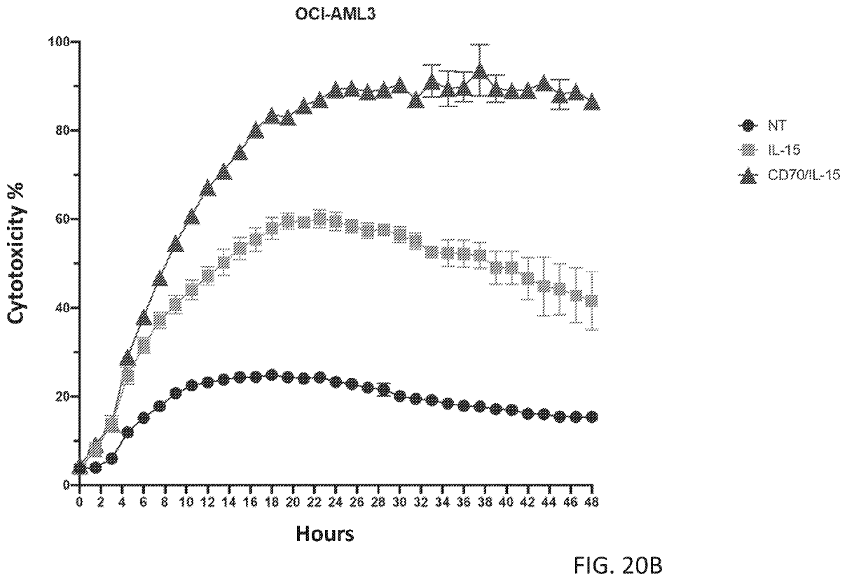

FIGS. 20A-20B show an IncuCyte® cytotoxicity assay on THP-1 and OCI-AML3 cells when cocultured with CBNK CD70 CAR cells. Compared to non-transduced (NT) cells, CBNK cells transduced with CD70 CAR showed increased cytotoxicity of THP-1 (FIG. 20A) and OCI-AML3 (FIG. 20B) cells, as shown by IncuCyte® assay, suggesting that CBNK CD70 CAR cells have greater killing activity against AML cells. CBNK cells transduced with IL15 construct was also used as a control in this assay, which shows enhanced cytotoxic activity compared to NT, but was not as effective as CD70 CAR.

Lung Cancer

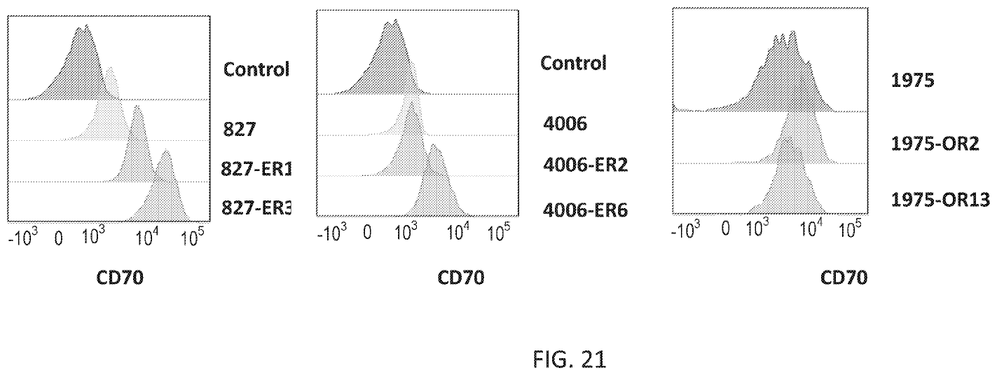

FIG. 21 shows expression of CD70 in various lung cancer cell lines. Surface expression of CD70 was detected in various lung cancer cell lines using flow cytometry.

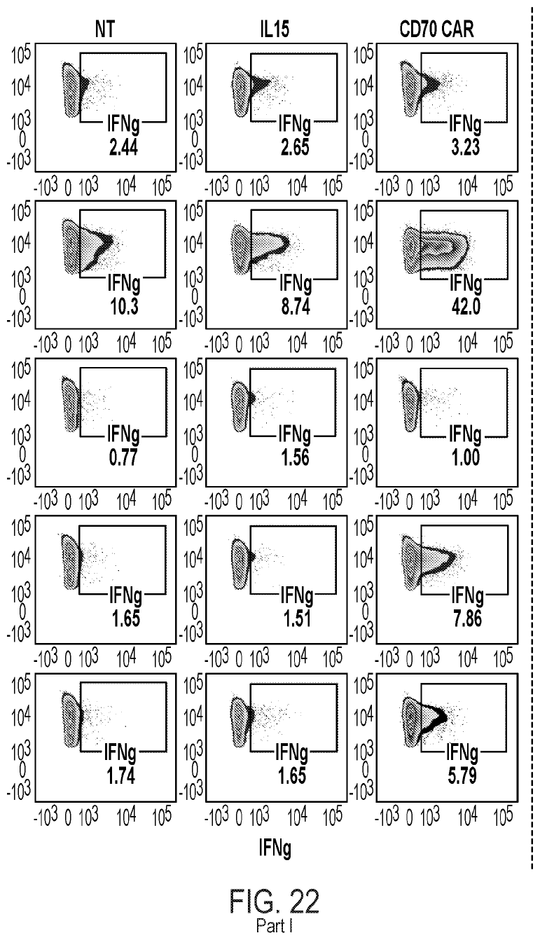

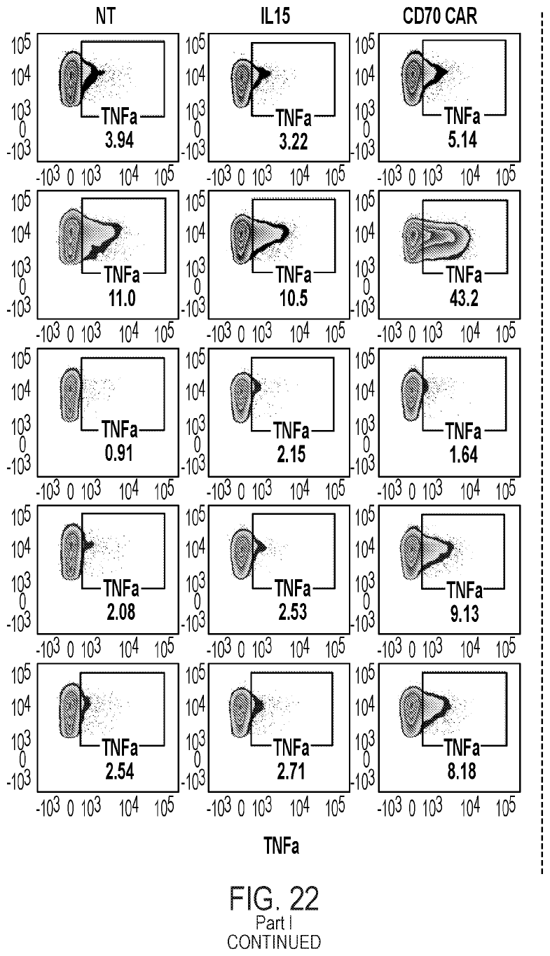

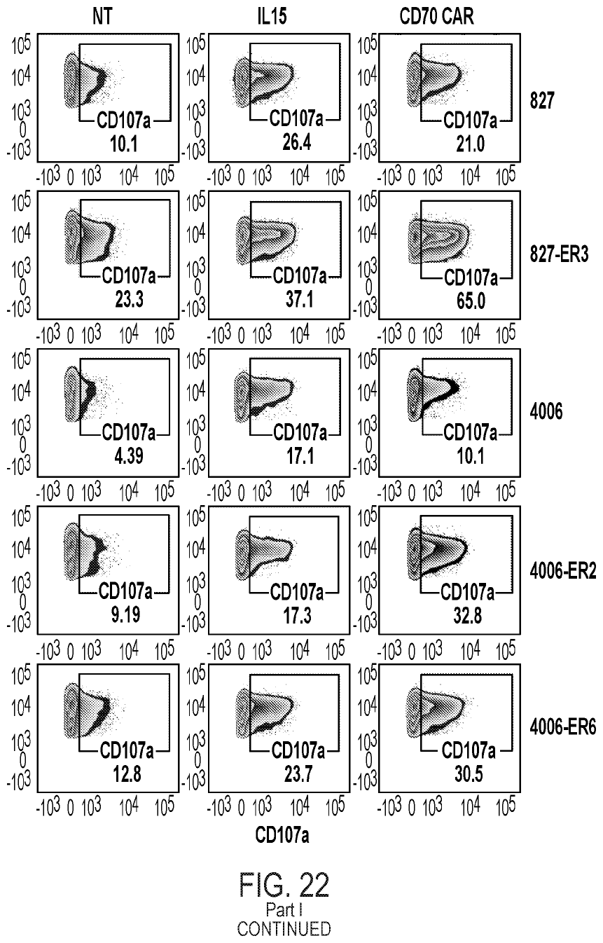

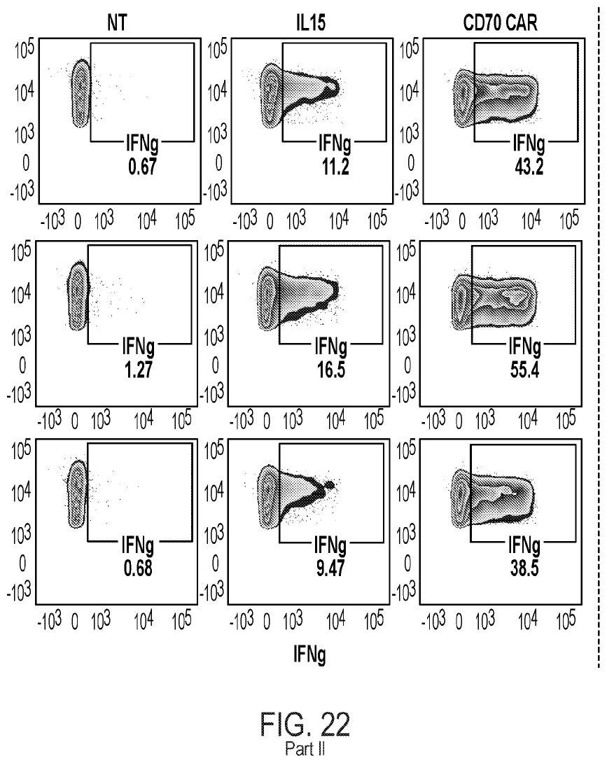

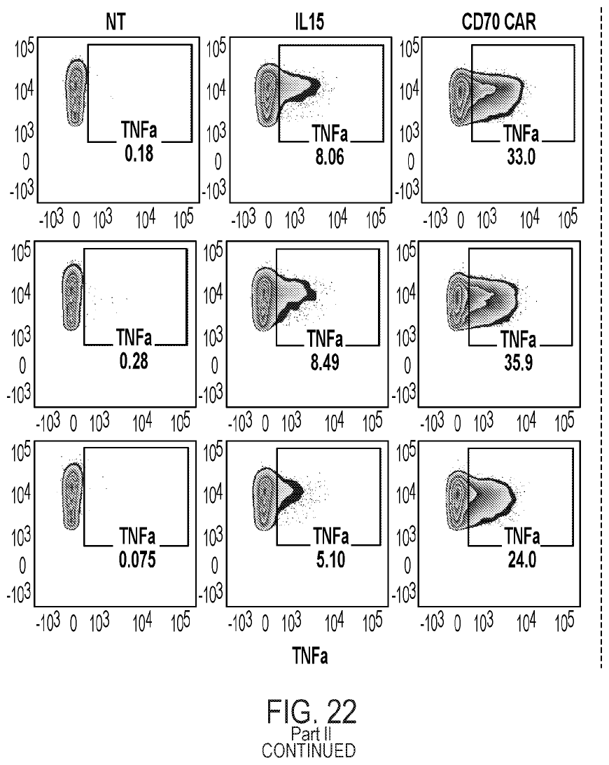

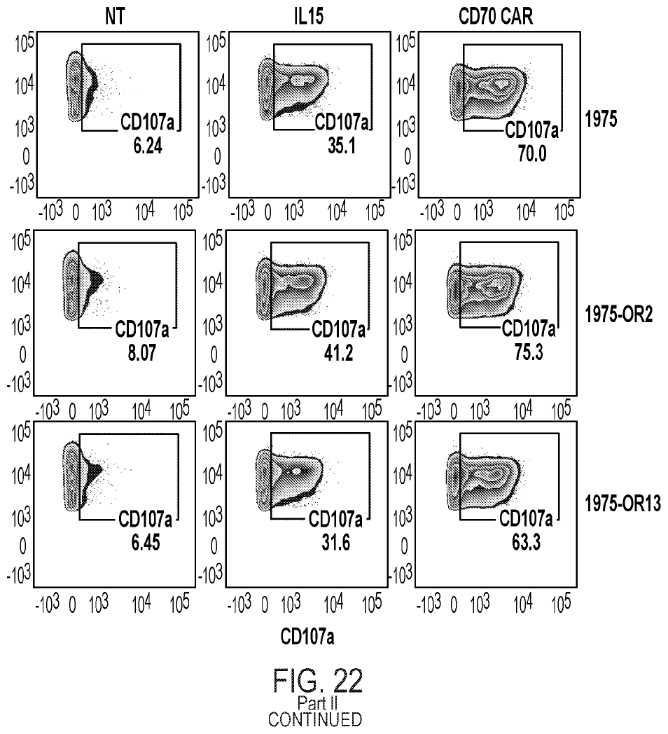

FIG. 22 shows intracellular cytokines and degranulation marker expression in CBNK CD70 CAR cells when co-cultured various lung cancer cell lines. Compared to non-transduced (NT) cells, CBNK cells transduced with CD70 CAR showed increased cytokines (interferon gamma and tumor necrosis factor alpha) secretion and degranulation marker CD107a expression when co-cultured with various lung cancer cell line, suggesting enhanced cytotoxic activity of CBNK CD70 CAR against lung cancer.

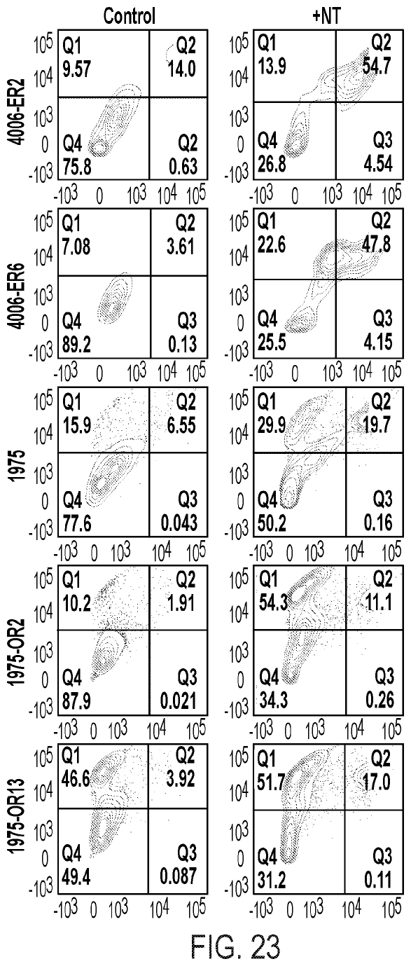

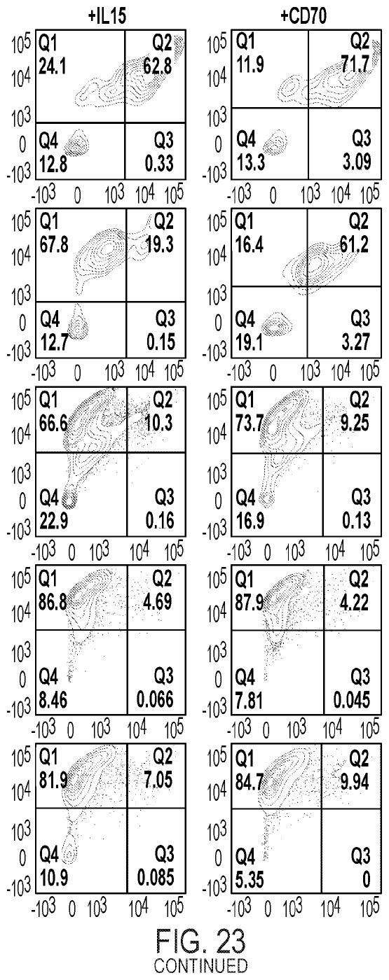

FIG. 23 demonstrates Annexin V staining to assess the apoptosis of lung cancer cells after co-culture with CBNK CD70 CAR cells. Compared to non-transduced (NT) cells, CBNK cells transduced with CD70 CAR showed increased apoptosis of various lung cancer cells, as shown by Annexin V-LIVE/DEAD™ Fixable Aqua staining assay, suggesting the enhanced cytotoxic activity of CBNK cells transduced with CD70 CAR against lung cancer cells.

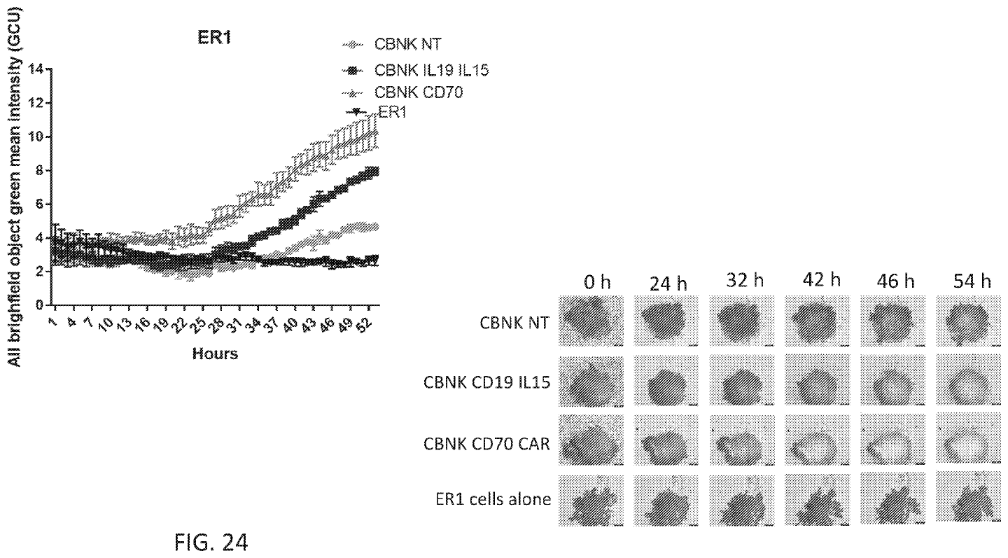

FIG. 24 demonstrates IncuCyt®e cytotoxicity assay on ER1 cells when cocultured with CBNK CD70 CAR cells. Compared to non-transduced (NT) cells, CBNK cells transduced with CD70 CAR showed increased cytotoxicity of ER1 cells, as shown by IncuCyte® assay, as assessed by the measurement of green (caspase 3/7) signal, suggesting that CBNK CD70 CAR cells have greater killing activity against lung cancer cells. CBNK cells transduced with CD19 CAR construct was also used as a control in this assay, which shows enhanced cytotoxic activity compared to NT, but was not as effective as CD70 CAR. Quantification of IncuCyte® cytotoxicity assay for 54 hours is shown in left panel, and representative images is shown in right panel.

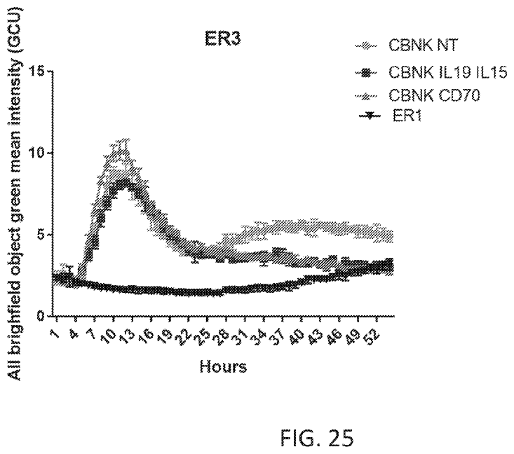

FIG. 25 shows IncuCyte® cytotoxicity assay on ER3 cells when cocultured with CBNK CD70 CAR cells. Compared to non-transduced (NT) cells, CBNK cells transduced with CD70 CAR showed increased cytotoxicity of ER3 cells, as shown by IncuCyte® assay, as assessed by the measurement of green (caspase 3/7) signal, suggesting that CBNK CD70 CAR cells have greater killing activity against lung cancer cells. CBNK cells transduced with CD19 CAR construct was also used as a control in this assay, which shows enhanced cytotoxic activity compared to NT, but was not as effective as CD70 CAR.

Breast Cancer

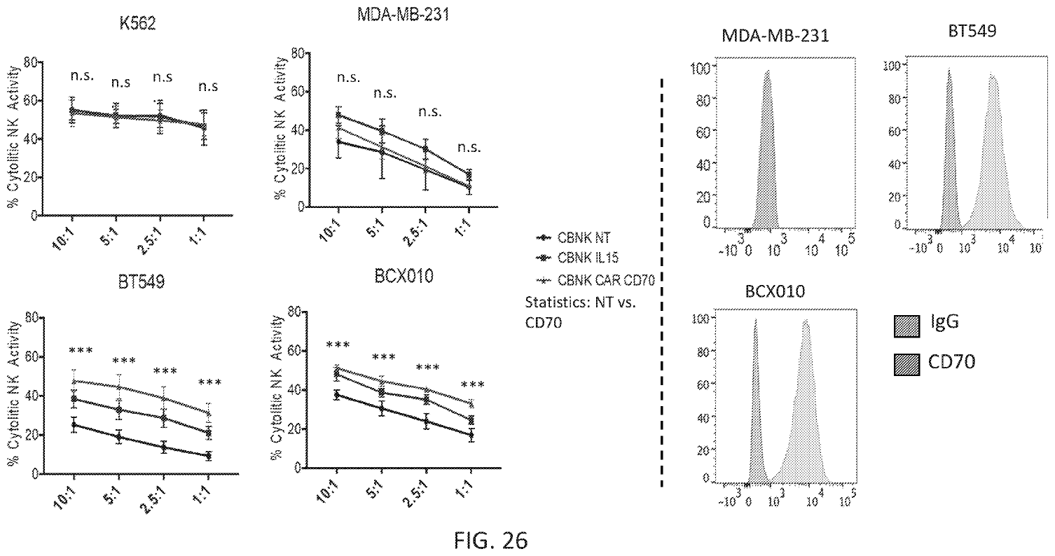

FIG. 26 shows a chromium release assay to assess the cytotoxic activity of CBNK CD70 CAR against breast cancer cell lines with varying CD70 expression. (Left) Surface expression of CD70 was detected in various breast cancer cell lines using flow cytometry. MBA-MB-231 has low/none CD70 expression, whereas BT549 and BCX010 have high CD70 expression. (Right) Compared to non-transduced (NT) cells, CBNK cells transduced with CD70 CAR showed increased cytotoxicity of BT549 and BCX010 cells, as shown by chromium release assay, suggesting that CBNK CD70 CAR cells have greater killing activity against breast cancer cells with high CD70 expression. K562 cells that are sensitive to NK cells are used as positive control. n.s. non significant; ***, P<0.001

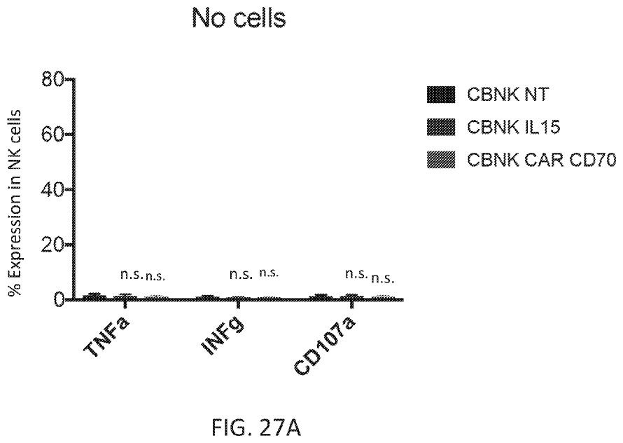

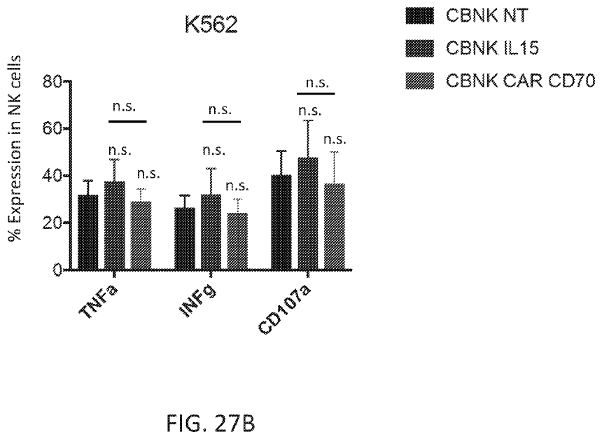

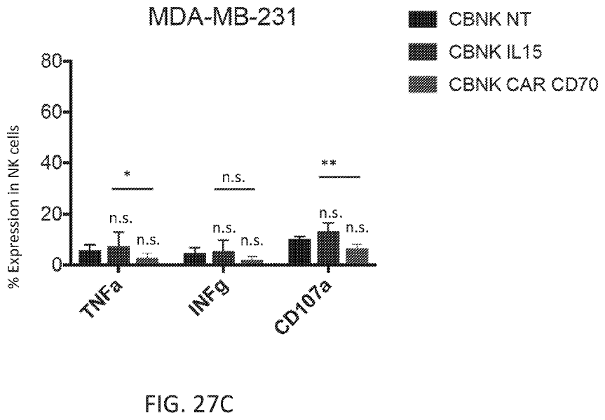

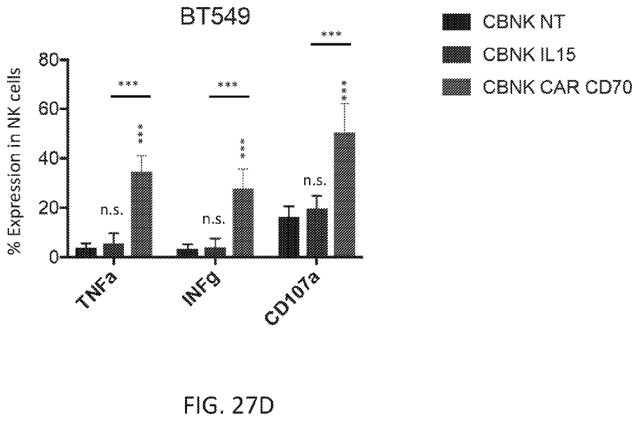

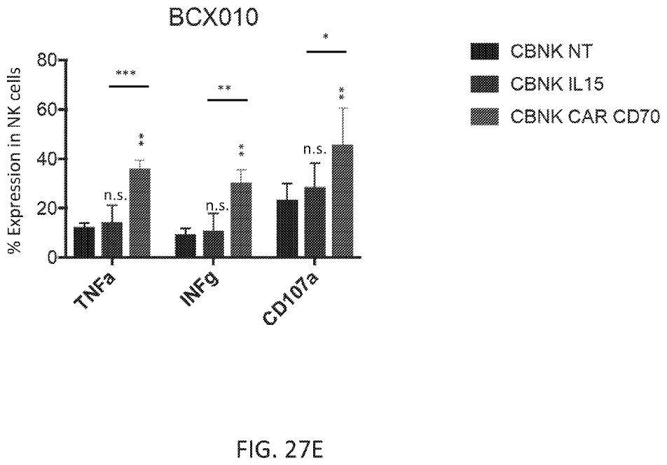

FIGS. 27A-27E show intracellular cytokines and degranulation marker expression in CBNK CD70 CAR cells when co-cultured with various breast cancer cells. Compared to non-transduced (NT) cells, CBNK cells transduced with CD70 CAR showed increased cytokines (interferon gamma and tumor necrosis factor alpha) secretion and degranulation marker CD107a expression when co-cultured with breast cancer cell lines with high CD70 surface expression, suggesting enhanced cytotoxic activity of CBNK CD70 CAR against breast cancer. n.s. non significant; *, p<0.05; **, p<0.01; ***, p<0.001.

Multiple Myeloma

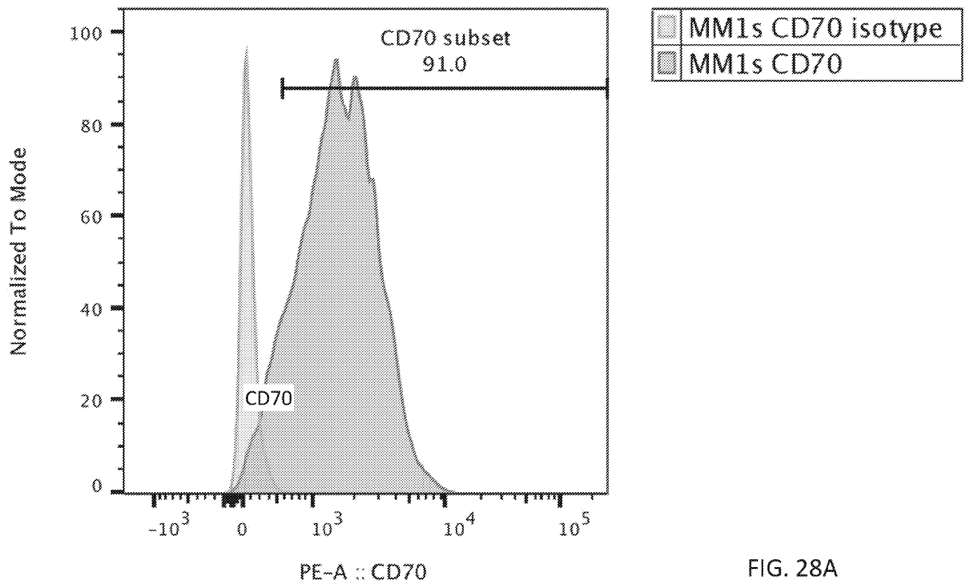

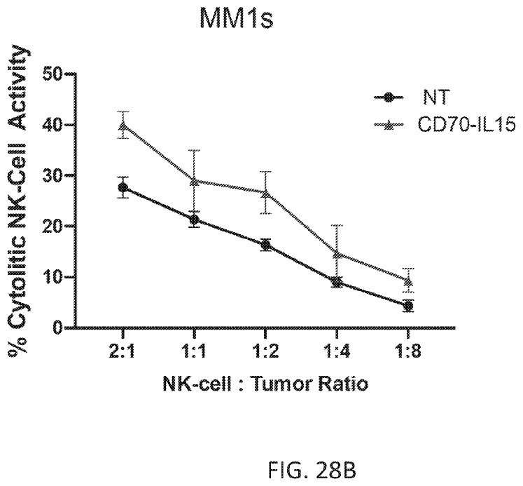

FIGS. 28A and 28B provide a chromium release assay to assess the cytotoxic activity of CBNK CD70 CAR against multiple myeloma. (FIG. 28A) Surface expression of CD70 was high in MM1s, a multiple myeloma cell lines, as detected by using flow cytometry. (FIG. 28B) Compared to non-transduced (NT) cells, CBNK cells transduced with CD70 CAR showed increased cytotoxicity of MM1s cells, as shown by chromium release assay, suggesting that CBNK CD70 CAR cells have greater killing activity against multiple myeloma cells.

Renal Cell Carcinoma (RCC)

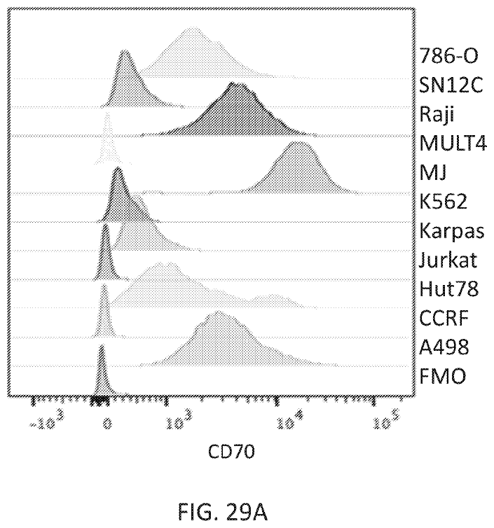

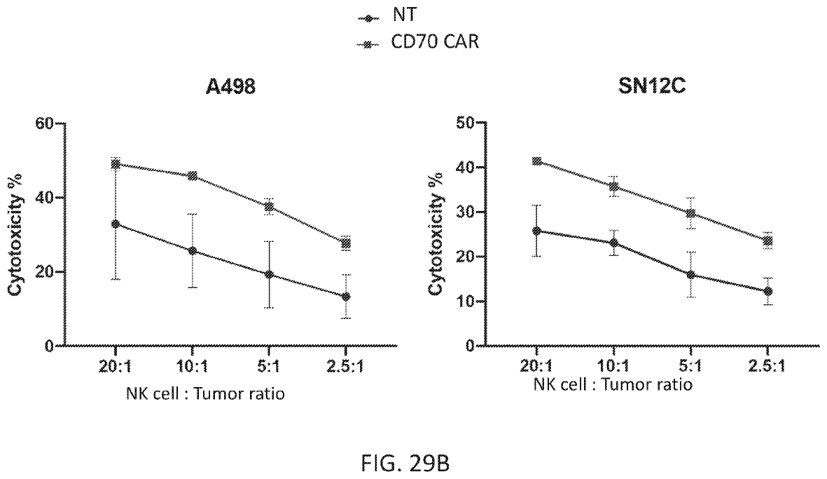

FIGS. 29A-29B show a chromium release assay to assess the cytotoxic activity of CBNK CD70 CAR against RCC. (FIG. 29A) Surface expression of CD70 was detected in various RCC and other cancer cell lines using flow cytometry. A498, SN12C and 786-0 are few RCC cell lines with high CD70 expression. (FIG. 29B) Compared to non-transduced (NT) cells, CBNK cells transduced with CD70 CAR showed increased cytotoxicity of A498 and SN12C cells, as shown by chromium release assay, suggesting that CBNK CD70 CAR cells have greater killing activity against RCC cells which have high CD70 expression.

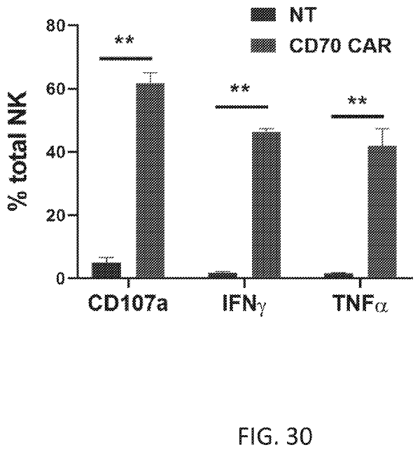

FIG. 30 shows production of intracellular cytokines and degranulation marker expression in CBNK CD70 CAR cells when co-cultured with RCC cells. Compared to non-transduced (NT) cells, CBNK cells transduced with CD70 CAR showed increased secretion of cytokines (interferon gamma and tumor necrosis factor alpha) and degranulation marker CD107a expression when co-cultured with RCC cell line 786-0 with high CD70 surface expression, suggesting enhanced cytotoxic activity of CBNK CD70 CAR against breast cancer. **, p<0.01

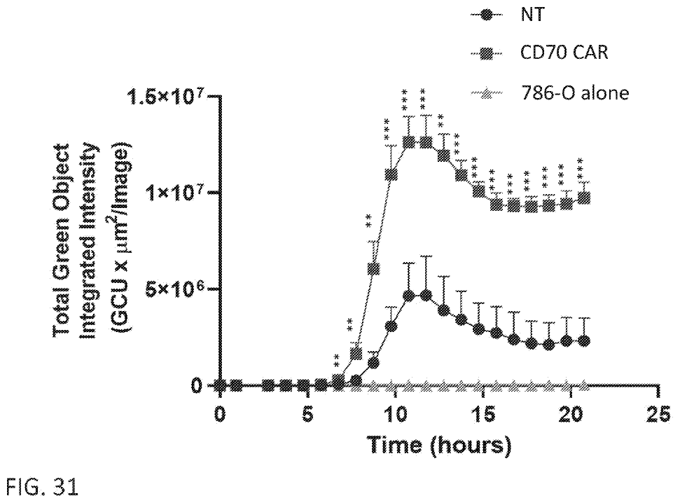

FIG. 31 shows IncuCyte® cytotoxicity assay on 786-0 RCC cells when cocultured with CBNK CD70 CAR cells. Compared to non-transduced (NT) cells, CBNK cells transduced with CD70 CAR showed increased cytotoxicity of 786-0 cells, as shown by IncuCyte® assay, as assessed by the measurement of green (caspase 3/7) signal, suggesting that CBNK CD70 CAR cells have greater killing activity against RCC. **, p<0.01; ***, p<0.001

Pancreatic Cancer

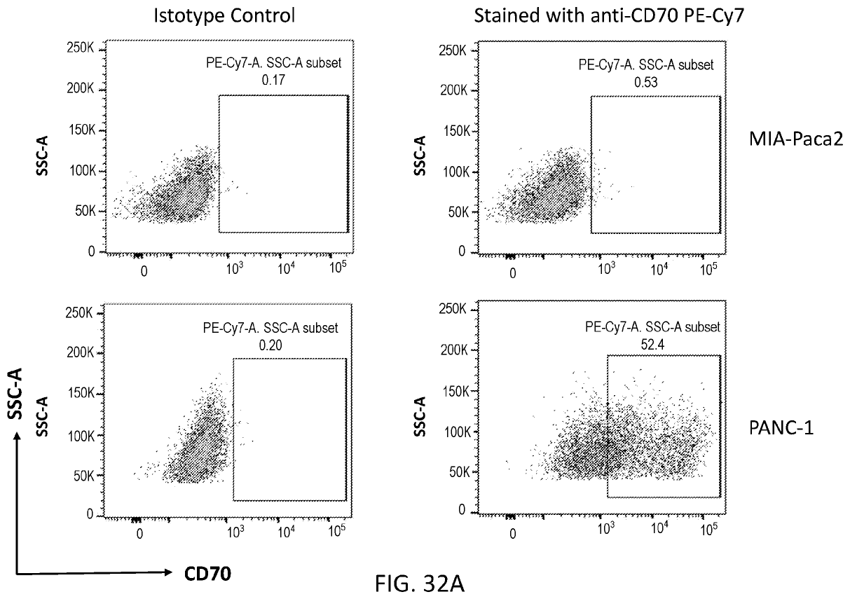

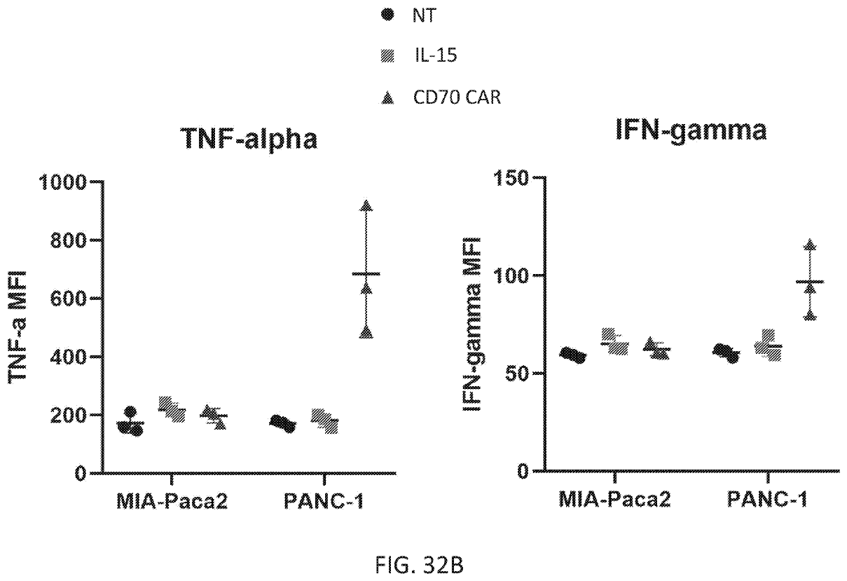

FIGS. 32A-32B show expression of intracellular cytokines in CBNK CD70 CAR cells when co-cultured with pancreatic cancer cells. (FIG. 32A) Surface expression of CD70 was detected in various pancreatic cancer cell lines using flow cytometry. MIA-Paca2 has low/non CD70 expression, whereas PANC-1 has high CD70 expression. (FIG. 32B) Compared to non-transduced (NT) cells, CBNK cells transduced with CD70 CAR showed increased cytokines (interferon gamma and tumor necrosis factor alpha) secretion when co-cultured with PANC-1 cell line (high CD70 expression) but not with MIA-Paca2 cell line (low CD70 expression), suggesting enhanced cytotoxic activity of CBNK CD70 CAR against pancreatic cells with high CD70 expression.

Glioblastoma (GBM)

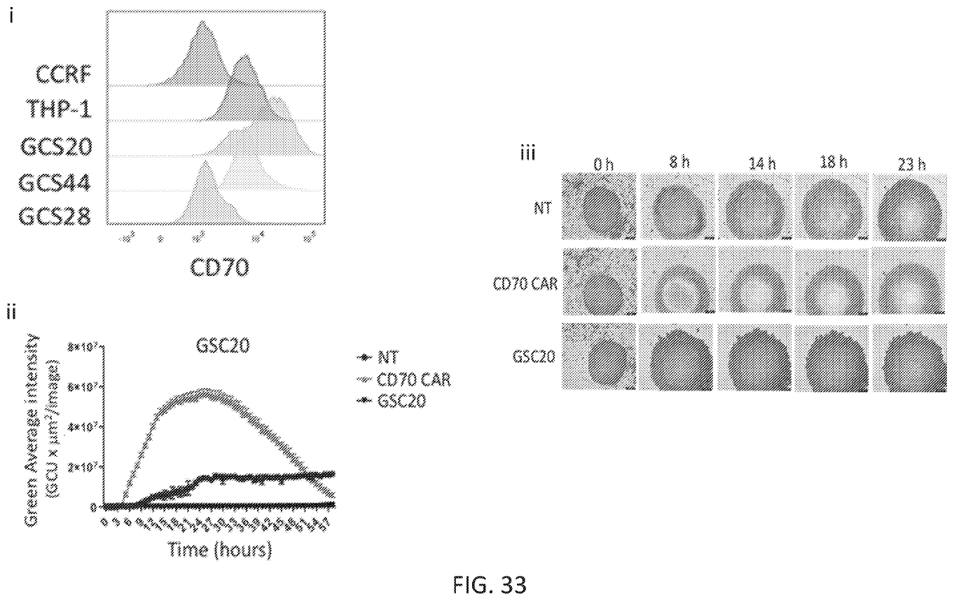

FIG. 33 demonstrates IncuCyte® cytotoxicity assay on GSC20 GBM cells when cocultured with CBNK CD70 CAR cells. Surface expression of CD70 was detected in various GBM cell lines using flow cytometry and GSC20 cell line showed the highest CD70 surface expression (panel i). Compared to non-transduced (NT) cells, CBNK cells transduced with CD70 CAR showed increased cytotoxicity of GSC20 cells, as shown by IncuCyte® assay, as assessed by the measurement of green (caspase 3/7) signal intensity, suggesting that CBNK CD70 CAR cells have greater killing activity against GBM cells. Quantification of IncuCyte® cytotoxicity assay for 57 hours is shown in panel ii, and representative images up to 23 hours is shown in panel iii.

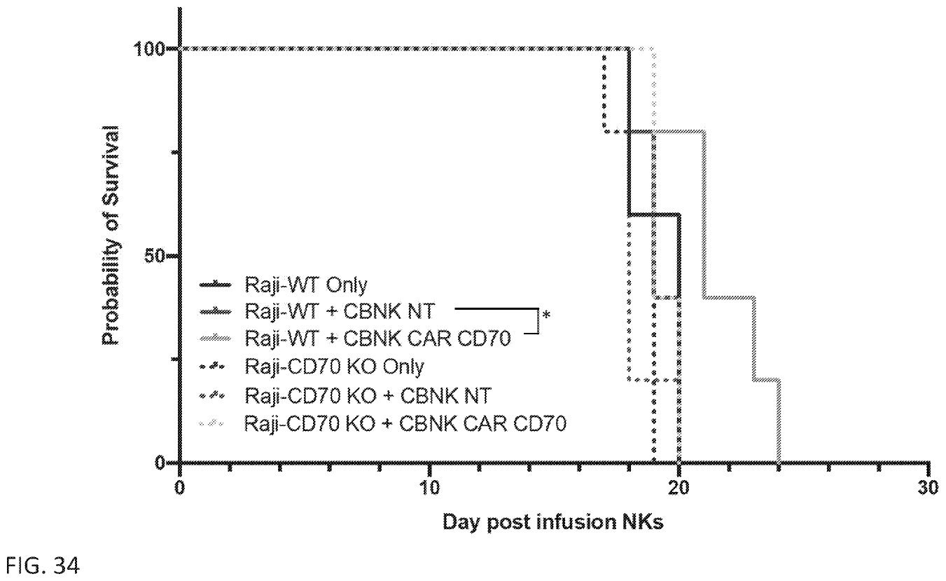

A survival curve of NSG mice (immunodeficient) engrafted with either Raji WT or CD70 KO cells and treated with CBNK CD70 CAR cells is provided in FIG. 34. Kaplan Meier plots demonstrate that CBNK cells transduced with CD70 CAR constructs shows improved survival in mice engrafted with Raji wild type (WT) tumor when compared to non-transduced CBNK cells. The improved survival was not seen in mice engrafted with CD70 knock out (KO) Raji cells, suggesting improved survival in mice is specific to CD70 antigen present in tumor cells.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. An expression construct comprising sequence that encodes a CD70-specific engineered receptor and that encodes one or both of the following:
(a) a suicide gene; and
(b) a cytokine.

2. The construct of claim 1, wherein the CD70-specific engineered receptor is a chimeric antigen receptor (CAR) or a T cell receptor.

3. The expression construct of claim 2, wherein the CD70-specific CAR comprises a scFv having a heavy chain and a light chain, and wherein the heavy chain in the sequence that encodes the CAR is upstream of the light chain in a 5′ to 3′ direction.

4. The expression construct of claim 2, wherein the CD70-specific CAR comprises a scFv having a heavy chain and a light chain, and wherein the heavy chain in the sequence that encodes the CAR is downstream of the light chain in a 5′ to 3′ direction.

5. The expression construct of any one of claims 1-4, wherein the CD70-specific CAR comprises a codon optimized scFv.

6. The expression construct of any one of claims 1-4, wherein the CD70-specific CAR comprises a humanized scFv.

7. The expression construct of any one of claims 1-6, wherein the CD70-specific CAR comprises a signaling peptide.

8. The expression construct of claim 7, wherein the signaling peptide is from CD8alpha, Ig heavy chain, or granulocyte-macrophage colony-stimulating factor receptor or a signal peptide derived from one or more other surface receptors.

9. The expression construct of any one of claims 1-8, wherein the CD70-specific CAR comprises one or more costimulatory domains.

10. The expression construct of claim 9, wherein the costimulatory domain is selected from the group consisting of CD28, CD27, OX-40 (CD134), DAP10, DAP12, 4-1BB (CD137), CD40L, 2B4, DNAM, CS1, CD48, NKG2D, NKp30, NKp44, NKp46, NKp80, and a combination thereof.

11. The expression construct of any one of claims 1-10, wherein the CD70-specific CAR comprises CD3zeta.

12. The expression construct of any one of claims 1-11, wherein the CD70-specific CAR comprises a hinge between the scFv and a transmembrane domain.

13. The expression construct of claim 12, wherein the hinge is CD8-alpha hinge, the hinge comprises an artificial spacer comprised of Gly3, or the hinge comprises CH1, CH2, and/or CH3 domains of IgGs.

14. The expression construct of any one of claims 1-13, wherein the cytokine is IL-15, IL-12, IL-2, IL-18, IL-21, IL-7, or a combination thereof.

15. The expression construct of any one of claims 1-14, wherein the suicide gene is a mutant TNF-alpha, inducible caspase 9, HSV-thymidine kinase, CD19, CD20, CD52, or EGFRv3.

16. The expression construct of claim 14, wherein the mutant TNF-alpha is an engineered nonsecretable mutant TNF-alpha.

17. An expression construct of any one of claims 1-16, wherein the expression construct comprises any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, or SEQ ID NO:13.

18. An immune cell, comprising the expression construct of any one of claims 1-17.

19. The immune cell of claim 18, wherein the immune cell is a natural killer (NK) cell, T cell, gamma delta T cells, invariant NKT (iNKT) cell, B cell, macrophage, MSCs, or dendritic cell.

20. The immune cell of claim 18 or 19, wherein the immune cell is a NK cell.

21. The immune cell of claim 19 or 20, wherein the NK cell is derived from cord blood, peripheral blood, induced pluripotent stem cells, bone marrow, or from a cell line.

22. The immune cell of claim 21, wherein the NK cell line is NK-92 cell line or another NK cell line derived from a tumor or from a healthy NK cell or a progenitor cell.

23. The immune cell of any one of claims 19-22, wherein the NK cell is a cord blood mononuclear cell.

24. The immune cell of any one of claims 19-23, wherein the NK cell is a CD56+NK cell.

25. The immune cell of any one of claims 19-24, wherein the NK cells express one or more exogenously provided cytokines.

26. The immune cell of claim 25, wherein the cytokine is IL-15, IL-2, IL-12, IL-18, IL-21, IL-7, or a combination thereof.

27. The immune cell of any one of claims 18-26, wherein expression of one or more endogenous genes in the immune cell has been modified.

28. The immune cell of claim 27, wherein the expression has been partially or fully reduced in expression.

29. The immune cell of claim 27 or 28, wherein expression of the one or more gene has been modified using CRISPR.

30. The immune cell of any one of claims 27-29, wherein the gene is selected from the group consisting of NKG2A, SIGLEC-7, LAGS, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, CD5, CD7, CTLA-4, TDAG8, CD38, and a combination thereof.

31. A population of immune cells of any one of claims 18-30, said cells present in a suitable medium.

32. The population of claim 31, wherein the immune cells are NK cells.

33. A method of killing CD70-positive cells in an individual, comprising the step of administering to the individual an effective amount of cells harboring the expression construct of any one of claims 1-17.

34. The method of claim 33, wherein the cells are NK cells, T cells, gamma delta T cells, induced NKT (iNKT) cells, B cells, macrophages, gamma delta T cells, or dendritic cells.

35. The method of claim 34, wherein the NK cells are derived from cord blood, peripheral blood, induced pluripotent stem cells, bone marrow, or from a cell line.

36. The method of any one of claims 34-35, wherein the NK cells are derived from cord blood mononuclear cells.

37. The method of any one of claims 33-36, wherein the CD70-positive cells are not cancer cells.

38. The method of claim 37, wherein the CD70-positive cells are T regulatory cells.

39. The method of any one of claims 33-36, wherein the individual has acute myeloid leukemia, lymphoma, lung cancer, renal cancer, bladder cancer, melanoma, glioblastoma, breast cancer, head and neck cancer, mesothelioma, multiple myeloma, pancreatic cancer or a combination thereof.

40. The method of any one of claims 33-39, wherein the cells are allogeneic with respect to the individual.

41. The method of any one of claims 33-39, wherein the cells are autologous with respect to the individual.

42. The method of any one of claims 33-41, wherein the individual is a human.

43. The method of any one of claims 32-42, wherein the cells are administered to the individual once or more than once.

44. The method of claim 43, wherein the duration of time between administrations of the cells to the individual is 1-24 hours, 1-7 days, 1-4 weeks, 1-12 months, or one or more years.

45. The method of any one of claims 33-44, further comprising the step of providing to the individual an effective amount of an additional therapy.

46. The method of claim 45, wherein the additional therapy comprises surgery, radiation, gene therapy, immunotherapy, or hormone therapy.

47. The method of claim 45 or 46, wherein the additional therapy comprises one or more antibodies.

48. The method of any one of claims 33-47, wherein the cells are administered to the individual by injection, intravenously, intraarterially, intraperitoneally, intratracheally, intratumorally, intramuscularly, endoscopically, intralesionally, intracranially, percutaneously, subcutaneously, regionally, by perfusion, in a tumor microenvironment, or a combination thereof.

49. The method of any one of claims 33-48, further comprising the step of identifying CD70-positive cells in the individual.

50. The method of any one of claims 33-49, further comprising the step of producing the cells harboring the expression construct.

51. As a composition of matter, the sequences of SEQ ID NO:1, SEQ ID NO: 2, SEQ ID NO:7, SEQ ID NO:8, SEQ ID NO:9, SEQ ID NO:10, SEQ ID NO:11, SEW NO:12, and SEQ ID NO:13.


SEQUENCE LISTING (13 sequences)

SEQ ID NO: 1 - DNA, 816 bp, Artificial Sequence
  Description of Artificial Sequence Synthetic polynucleotide
atggccctgcctgtgacagctctgctcctccctctggccctgctgctccatgccgccagaccccaggcagttgtgacccaggagccttccctgacagtgtctccaggagggacggtcacgctcacctgcggcctcaaatctgggtctgtcacttccgataacttccccacttggtaccagcagacaccaggccaggctccccgattgcttatctacaacacaaacacccgtcactctggcgtccccgaccgcttctccggatccatcctgggcaacaaagccgccctcaccatcacgggggcccaggccgacgacgaggccgaatatttctgtgctctgttcataagtaatcctagtgttgagttcggcggagggacccaactgaccgtcctaggtggcagcaccagcggctccggcaagcctggctctggcgagggcagcacaaagggagaggtgcagctcgtggagtctgggggaggcttggtgcagcctggggggtctctgagactctcctgtgcagcctctggattcaccttcagtgtctactacatgaactgggtccgccaggctccagggaaggggctcgagtgggtctcagatattaataatgaaggtggtactacatactatgcagactccgtgaagggccgattcaccatctccagagacaactctaagaacagcctgtatctgcaaatgaacagcctgcgcgccgaggacacggccgtgtactactgcgcgagagatgccggatatagcaaccatgtacccatctttgattcttggggccaggggaccctggtcactgtctcctca

SEQ ID NO: 2 - protein, 272 aa, Artificial Sequence
  Description of Artificial Sequence Synthetic polypeptide
MALPVTALLLPLALLLHAARPQAVVTQEPSLTVSPGGTVTLTCGLKSGSVTSDNFPTWYQQTPGQAPRLLIYNTNTRHSGVPDRFSGSILGNKAALTITGAQADDEAEYFCALFISNPSVEFGGGTQLTVLGGSTSGSGKPGSGEGSTKGEVQLVESGGGLVQPGGSLRLSCAASGFTFSVYYMNWVRQAPGKGLEWVSDINNEGGTTYYADSVKGRFTISRDNSKNSLYLQMNSLRAEDTAVYYCARDAGYSNHVPIFDSWGQGTLVTVSS

SEQ ID NO: 3 - DNA, 816 bp, Artificial Sequence
  Description of Artificial Sequence Synthetic polynucleotide
atggccctgcctgtgacagctctgctcctccctctggccctgctgctccatgccgccagacccgaggtgcagctcgtggagtctgggggaggcttggtgcagcctggggggtctctgagactctcctgtgcagcctctggattcaccttcagtgtctactacatgaactgggtccgccaggctccagggaaggggctcgagtgggtctcagatattaataatgaaggtggtactacatactatgcagactccgtgaagggccgattcaccatctccagagacaactctaagaacagcctgtatctgcaaatgaacagcctgcgcgccgaggacacggccgtgtactactgcgcgagagatgccggatatagcaaccatgtacccatctttgattcttggggccaggggaccctggtcactgtctcctcaggcagcaccagcggctccggcaagcctggctctggcgagggcagcacaaagggacaggcagtggtgacccaggagccttccctgacagtgtctccaggagggacggtcacgctcacctgcggcctcaaatctgggtctgtcacttccgataacttccccacttggtaccagcagacaccaggccaggctccccgattgcttatctacaacacaaacacccgtcactctggcgtccccgaccgcttctccggatccatcctgggcaacaaagccgccctcaccatcacgggggcccaggccgacgacgaggccgaatatttctgtgctctgttcataagtaatcctagtgttgagttcggcggagggacccaactgaccgtcctaggt

SEQ ID NO: 4 - protein, 274 aa, Artificial Sequence
  Description of Artificial Sequence Synthetic polypeptide
MGMALPVTALLLPLALLLHAARPEVQLVESGGGLVQPGGSLRLSCAASGFTFSVYYMNWVRQAPGKGLEWVSDINNEGGTTYYADSVKGRFTISRDNSKNSLYLQMNSLRAEDTAVYYCARDAGYSNHVPIFDSWGQGTLVTVSSGSTSGSGKPGSGEGSTKGQAVVTQEPSLTVSPGGTVTLTCGLKSGSVTSDNFPTWYQQTPGQAPRLLIYNTNTRHSGVPDRFSGSILGNKAALTITGAQADDEAEYFCALFISNPSVEFGGGTQLTVLG

SEQ ID NO: 5 - DNA, 816 bp, Artificial Sequence
  Description of Artificial Sequence Synthetic polynucleotide
atggccctgcctgtgacagctctgctcctccctctggccctgctgctccatgccgccagacccgaggtgcagctcgtggagtctgggggaggcttggtgcagcctggggggtctctgagactctcctgtgcagcctctggattcaccttcagtgtctactacatgaactgggtccgccaggctccagggaaggggcttgagtgggtctcagatattaataatgaaggtggtactacatactatgcagactccgtgaagggccgattcaccatctccagagacaactctaagaacagcctgtatctgcaaatgaacagcctgcgcgccgaggacacggccgtgtactactgcgcgagagatgccggatatagcaaccatgtacccatctttgattcttggggccaggggaccctggtcactgtctcctcaggcagcaccagcggctccggcaagcctggctctggcgagggcagcacaaagggacaggcagtggtgacccaggagccttccctgacagtgtctccaggagggacggtcacgctcacctgcggcctcaaatctgggtctgtcacttccgataacttccccacttggtaccagcagacaccaggccaggctccccgattgcttatctacaacacaaacacccgtcactctggcgtccccgaccgcttctccggatccatcctgggcaacaaagccgccctcaccatcacgggggcccaggccgacgacgaggccgaatatttctgtgctctgttcataagtaatcctagtgttgagttcggcggagggacccaactgaccgtcctaggt

SEQ ID NO: 6 - protein, 274 aa, Artificial Sequence; identical to SEQ ID NO: 4
  Description of Artificial Sequence Synthetic polypeptide

SEQ ID NO: 7 - DNA, 2652 bp, Artificial Sequence
  Description of Artificial Sequence Synthetic polynucleotide
atggccctgcctgtgacagctctgctcctccctctggccctgctgctccatgccgccagaccccaggcagttgtgacccaggagccttccctgacagtgtctccaggagggacggtcacgctcacctgcggcctcaaatctgggtctgtcacttccgataacttccccacttggtaccagcagacaccaggccaggctccccgattgcttatctacaacacaaacacccgtcactctggcgtccccgaccgcttctccggatccatcctgggcaacaaagccgccctcaccatcacgggggcccaggccgacgacgaggccgaatatttctgtgctctgttcataagtaatcctagtgttgagttcggcggagggacccaactgaccgtcctaggtggcagcaccagcggctccggcaagcctggctctggcgagggcagcacaaagggagaggtgcagctcgtggagtctgggggaggcttggtgcagcctggggggtctctgagactctcctgtgcagcctctggattcaccttcagtgtctactacatgaactgggtccgccaggctccagggaaggggctcgagtgggtctcagatattaataatgaaggtggtactacatactatgcagactccgtgaagggccgattcaccatctccagagacaactctaagaacagcctgtatctgcaaatgaacagcctgcgcgccgaggacacggccgtgtactactgcgcgagagatgccggatatagcaaccatgtacccatctttgattcttggggccaggggaccctggtcactgtctcctcacgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 8 - DNA, 2652 bp, Artificial Sequence
  Description of Artificial Sequence Synthetic polynucleotide
atggccctgcctgtgacagctctgctcctccctctggccctgctgctccatgccgccagacccgaggtgcagctcgtggagtctgggggaggcttggtgcagcctggggggtctctgagactctcctgtgcagcctctggattcaccttcagtgtctactacatgaactgggtccgccaggctccagggaaggggctcgagtgggtctcagatattaataatgaaggtggtactacatactatgcagactccgtgaagggccgattcaccatctccagagacaactctaagaacagcctgtatctgcaaatgaacagcctgcgcgccgaggacacggccgtgtactactgcgcgagagatgccggatatagcaaccatgtacccatctttgattcttggggccaggggaccctggtcactgtctcctcaggcagcaccagcggctccggcaagcctggctctggcgagggcagcacaaagggacaggcagtggtgacccaggagccttccctgacagtgtctccaggagggacggtcacgctcacctgcggcctcaaatctgggtctgtcacttccgataacttccccacttggtaccagcagacaccaggccaggctccccgattgcttatctacaacacaaacacccgtcactctggcgtccccgaccgcttctccggatccatcctgggcaacaaagccgccctcaccatcacgggggcccaggccgacgacgaggccgaatatttctgtgctctgttcataagtaatcctagtgttgagttcggcggagggacccaactgaccgtcctaggtcgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 9 - DNA, 2652 bp, Artificial Sequence
  Description of Artificial Sequence Synthetic polynucleotide
atggccctgcctgtgacagctctgctcctccctctggccctgctgctccatgccgccagaccccaggcagttgtgacccaggagccttccctgacagtgtctccaggagggacggtcacgctcacctgcggcctcaaatctgggtctgtcacttccgataacttccccacttggtaccagcagacaccaggccaggctccccgattgcttatctacaacacaaacacccgtcactctggcgtccccgaccgcttctccggatccatcctgggcaacaaagccgccctcaccatcacgggggcccaggccgacgacgaggccgaatatttctgtgctctgttcataagtaatcctagtgttgagttcggcggagggacccaactgaccgtcctaggtggcagcaccagcggctccggcaagcctggctctggcgagggcagcacaaagggagaggtgcagctcgtggagtctgggggaggcttggtgcagcctggggggtctctgagactctcctgtgcagcctctggattcaccttcagtgtctactacatgaactgggtccgccaggctccagggaaggggcttgagtgggtctcagatattaataatgaaggtggtactacatactatgcagactccgtgaagggccgattcaccatctccagagacaactctaagaacagcctgtatctgcaaatgaacagcctgcgcgccgaggacacggccgtgtactactgcgcgagagatgccggatatagcaaccatgtacccatctttgattcttggggccaggggaccctggtcactgtctcctcacgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 10 - DNA, 816 bp, Artificial Sequence
  Description of Artificial Sequence Synthetic polynucleotide
atggccctgcctgtgacagctctgctcctccctctggccctgctgctccatgccgccagaccccaggcagtggtgacccaggagccttccctgacagtgtctccaggagggacggtcacgctcacctgcggcctcaaatctgggtctgtcacttccgataacttccccacttggtaccagcagacaccaggccaggctccccgattgcttatctacaacacaaacacccgtcactctggcgtccccgaccgcttctccggatccatcctgggcaacaaagccgccctcaccatcacgggggcccaggccgacgacgaggccgaatatttctgtgctctgttcataagtaatcctagtgttgagttcggcggagggacccaactgaccgtcctaggtggcagcaccagcggctccggcaagcctggctctggcgagggcagcacaaagggagaggtgcagctcgtggagtctgggggaggcttggtgcagcctggggggtctctgagactctcctgtgcagcctctggattcaccttcagtgtctactacatgaactgggtccgccaggctccagggaaggggctcgagtgggtctcagatattaataatgaaggtggtactacatactatgcagactccgtgaagggccgattcaccatctccagagacaactctaagaacagcctgtatctgcaaatgaacagcctgcgcgccgaggacacggccgtgtactactgcgcgagagatgccggatatagcaaccatgtacccatctttgattcttggggccaggggaccctggtcactgtctcctca

SEQ ID NO: 11 - protein, 279 aa, Artificial Sequence
  Description of Artificial Sequence Synthetic polypeptide
ARVATMGMALPVTALLLPLALLLHAARPQAVVTQEPSLTVSPGGTVTLTCGLKSGSVTSDNFPTWYQQTPGQAPRLLIYNTNTRHSGVPDRFSGSILGNKAALTITGAQADDEAEYFCALFISNPSVEFGGGTQLTVLGGSTSGSGKPGSGEGSTKGEVQLVESGGGLVQPGGSLRLSCAASGFTFSVYYMNWVRQAPGKGLEWVSDINNEGGTTYYADSVKGRFTISRDNSKNSLYLQMNSLRAEDTAVYYCARDAGYSNHVPIFDSWGQGTLVTVSS

SEQ ID NO: 12 - DNA, 2652 bp, Artificial Sequence; identical to SEQ ID NO: 9
  Description of Artificial Sequence Synthetic polynucleotide

SEQ ID NO: 13 - DNA, 2652 bp, Artificial Sequence
  Description of Artificial Sequence Synthetic polynucleotide
atggccctgcctgtgacagctctgctcctccctctggccctgctgctccatgccgccagacccgaggtgcagctcgtggagtctgggggaggcttggtgcagcctggggggtctctgagactctcctgtgcagcctctggattcaccttcagtgtctactacatgaactgggtccgccaggctccagggaaggggcttgagtgggtctcagatattaataatgaaggtggtactacatactatgcagactccgtgaagggccgattcaccatctccagagacaactctaagaacagcctgtatctgcaaatgaacagcctgcgcgccgaggacacggccgtgtactactgcgcgagagatgccggatatagcaaccatgtacccatctttgattcttggggccaggggaccctggtcactgtctcctcaggcagcaccagcggctccggcaagcctggctctggcgagggcagcacaaagggacaggcagtggtgacccaggagccttccctgacagtgtctccaggagggacggtcacgctcacctgcggcctcaaatctgggtctgtcacttccgataacttccccacttggtaccagcagacaccaggccaggctccccgattgcttatctacaacacaaacacccgtcactctggcgtccccgaccgcttctccggatccatcctgggcaacaaagccgccctcaccatcacgggggcccaggccgacgacgaggccgaatatttctgtgctctgttcataagtaatcctagtgttgagttcggcggagggacccaactgaccgtcctaggtcgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga